(19) United States
(12) Patent Application Publication
(10) Pub. No.: US 2026/0062408 A1
(43) Pub. Date: Mar. 5, 2026

(54) PRODRUGS OF SUBSTITUTED ERGOLINES

(71) Applicant: SYNEX HOLDINGS BV, MAASTRICHT (NL)

(72) Inventors: ALEXANDER STRATFORD, MAASTRICHT (NL); JAMES PETER BERNARD WILLIAMSON, MAASTRICHT (NL)

(21) Appl. No.: 19/101,055

(22) PCT Filed: Aug. 4, 2023
(86) PCT No.: PCT/EP2023/071722

§ 371 Date: Feb. 4, 2025

(30) Foreign Application Priority Data
Aug. 4, 2022 (EP) 22188643.5
Aug. 16, 2022 (EP) 22190617.5

(51) Int. Cl. C07D 457/06; A61K 31/48; A61K 31/5377; A61K 31/695; A61P 25/00; C07D 457/08; C07D 519/00; C07F 7/08

(52) U.S. Cl. CPC C07D 457/06; A61K 31/48; A61K 31/5377; A61K 31/695; A61P 25/00; C07D 457/08; C07D 519/00; C07F 7/0812

(57) ABSTRACT

The present invention relates generally to substituted ergoline compounds and compositions, more particularly new lysergamide derivatives and compositions, and applications of these. The invention furthermore relates to a new production process for 2-bromo lysergamide derivatives and intermediates of said production process.

### FIELD OF THE INVENTION

The present invention relates generally to substituted ergoline compounds and compositions, more particularly new lysergamide derivatives and compositions, and applications of these. The invention furthermore relates to a new production process for 2-bromo lysergamide derivatives and intermediates of said production process.

### BACKGROUND OF THE INVENTION

Ergolines are a class of bioactive chemical compounds originally isolated from the fungus Claviceps purpurea which grows on rye. Ergoline derivatives have a long history of medical use for a variety of applications, particularly in obstetrics.

Most medically useful drugs of this type are lysergic acid amide derivatives or “lysergamides”. Many compounds of this type show potent activity as agonists or antagonists for G-protein coupled receptors normally activated by the neurotransmitters serotonin and dopamine. Selectivity for various different receptor subtypes can be achieved through different structural modifications, with especially prominent activity at the serotonin 5-HT_{1A}, 5-HT_{2A}, 5-HT_{2c} and dopamine D₄ receptors (Passie T, et al. CNS Neurosci Ther. 2008; 14(4):295-314. doi: 10.1111/j.1755-5949.2008.00059.x).

Previously, LSD was used successfully to treat mental conditions such as alcoholism (Krebs T S, et al. J Psychopharmacol. 2012 July; 26(7):994-1002. doi: 10.1177/0269881112439253). As LSD therapy peaked in the late 1950s and early 1960s, it was widely considered as a revolutionary therapy in psychiatry. Unfortunately, LSD increasingly came to be viewed as a drug of abuse by governments. Young people were using it for its recreational effects and, as a consequence, it was banned by many nations and abandoned as a therapeutic tool for around half a century (Nichols D E. ACS Chem Neurosci. 2018 Oct. 17; 9(10):2331-2343. doi: 10.1021/acschemneuro.8b00043). However, more recently, LSD has again gained attention and pilot studies have confirmed its efficacy as a tool in psychotherapy (Liechti M E. Neuropsychopharmacology. 2017 October; 42(11):2114-2127. doi: 10.1038/npp.2017.86). In a supportive setting, significant reduction of symptoms of anxiety and depression following LSD-assisted psychotherapy sessions have been reported, with a rapid onset of action and reduction of symptoms lasting for up to 12 months after only two LSD-assisted therapy sessions (Muttoni S, et al. J Affect Disord. 2019 Nov. 1; 258:11-24. doi: 10.1016/j.jad.2019.07.076).

Another application for which lysergamide derivatives have received considerable attention is in the treatment of cluster headaches, a debilitating condition which is often unresponsive to conventional treatment options. Surveys of cluster headache sufferers have provided evidence that LSD, 2-bromo-LSD and lysergic acid amide are comparable to or more effective than conventional medications (Schindler E A D, et al., J Psychoactive Drugs. 2015; 47(5):372-81. doi: 10.1080/02791072.2015.1107664), however no such compounds have yet been officially approved for this application and research remains at a preliminary stage. Numerous additional applications are also known for which 5-HT2A agonist compounds have been demonstrated to show therapeutic use. Examples of these include the treatment of ocular conditions including glaucoma (May J A, et al. J Med Chem. 2015 Nov. 25; 58(22):8818-33. doi: 10.1021/acs.jmedchem.5b00857), and conditions associated with pathological ocular neovascularization such as macular degeneration (Foster T P, et al. US Patent Application 2020/0330405 A1). Another example is the application of 5-HT_{2A} agonist compounds in inhibiting cellular signalling mediated by TNF_{α}, which can be of use in the treatment of inflammatory disorders such as rheumatoid arthritis (Yu B, et al. J Pharmacol Exp Ther. 2008 November; 327(2):316-23. doi: 10.1124/jpet.108.143461), and Alzheimer's disease (Family N, et al. Psychopharmacology (Berl). 2020 March; 237(3):841-853. doi: 10.1007/s00213-019-05417-7). It may be envisaged that ergoline derivatives such as the compounds of the invention which possess 5-HT_{2A} agonist activity, may also be used for the treatment of the above listed conditions and related conditions resulting from similar underlying pathology.

Despite the positive results reported to date, there is a need to provide alternatives to known lysergamide compounds. The hallucinogenic side effects of LSD can limit its applicability in the treatment of anxiety and depression, while conversely the non-psychoactive 2-bromo-LSD is less effective than LSD itself in the treatment of cluster headaches.

Some 1-acyl lysergamide derivatives are already known to those skilled in the art to which the invention relates. 1-acetyl-d-lysergic acid diethylamide (1-acetyl-LSD, also known as ALD-52) was first described in the 1950s, e.g. its synthesis and that of 1-acetyl-lysergic acid monoethylamide is described in U.S. Pat. No. 2,810,723. More recently several other lysergamide derivatives with small acyl groups substituted on the 1-position have been described, notably 1-propionoyl-lysergic acid diethylamide (1P-LSD), 1-butanoyl-lysergic acid diethylamide (1B-LSD), 1-cyclopropylcarbonyl-lysergic acid diethylamide (1 cP-LSD), 1-valeroyl-lysergic acid diethylamide (1V-LSD), 1-propionoyl-6-allyl-6-nor-lysergic acid diethylamide (1P-AL-LAD), 1-cyclopropanecarbonyl-6-allyl-6-nor-lysergic acid diethylamide (1cP-AL-LAD), 1-propionoyl-6-ethyl-6-nor-lysergic acid diethylamide (1P-ETH-LAD), 1-propionyl-lysergic acid methylisopropylamide (1P-MIPLA) and 1-cyclopropanecarbonyl-lysergic acid methylisopropylamide (1 cP-MIPLA) (S. D. Brandt et al, Drug Test Analysis (2015), DOI 10.1002/dta.1884; S. D. Brandt et al, Drug Test Analysis (2019), DOI 10.1002/dta.2613; S. D. Brandt et al., Drug Test Analysis (2020), DOI 10.1002/dta.2789; S. D. Brandt et al., Drug Test Analysis (2022), DOI 10.1002/dta.3281; S. D. Brandt et al., Drug Test Analysis (2021), DOI 10.1002/dta.3205; S. D. Brandt et al., Drug Test Analysis (2017), DOI 10.1002/dta.2196; Christina Grumann et al., Drug Test Analysis (2020), DOI 10.1002/dta.2821; Christina Grumann et al., Journal of Pharmaceutical and Biomedical Analysis (2019), DOI 10.1016/j.jpba.2019.05.062; Adam L. Halberstadt et al., Neuropharmacology (2020), DOI 10.1016/j.neuropharm.2019.107856; and Lea Wagmann et al., Analytical and Bioanalytical Chemistry (2019), DOI 10.1007/s00216-018-1558-9). In all cases, these lysergamide derivatives have a small 1-acyl group with 5 carbons or less and their relevant pharmacokinetic and pharmacodynamic properties, notably their hallucinogenic side effects compared to the previously known compounds LSD and ALD-52, have not been described so far.

In an attempt to provide ergoline derivatives with modulated/reduced hallucinogenic side effects, it was found that a wide range of 1-acyl, 1-carbamate and 1-sulfonyl derivatives of ergoline exhibit improved pharmacokinetic and pharmacodynamic properties, such as reduced hallucinogenic side effects, compared to previously described compounds LSD and ALD-52, which makes them suitable for broad variety of therapeutic applications. Moreover, it has been found that the respective 1-acyl-, 1-carbamate- and 1-sulfonyl-derivatives of 2-bromo-lysergamides can simply be prepared from bromocriptine.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

In a first aspect, the present invention provides a compound of formula (1),

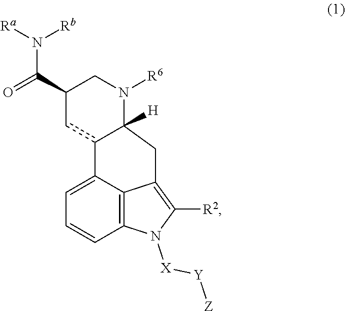

wherein
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C₁₋₁₂ alkylthio, aryl, heterocyclyl, heteroaryl, metallocenyl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, alkylthio, cycloalkyl, cycloalkoxy, aryl, heterocyclyl, heteroaryl or metallocenyl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂;
R² is selected from hydrogen, C₁₋₃ alkyl, C₃₋₅ cycloalkyl, oxetane, C₁₋₃ haloalkyl, C₁₋₃ alkylthio, halogen, cyano, COOH and CONH₂;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅alkenyl, C₂₋₅ alkynyl, C₃₋₇ cycloalkyl, C₂₋₄ haloalkyl, 2-methoxyethyl and 2-ethoxyethyl; or R^{a} and R^{b}, together with the adjacent nitrogen atom, form a 3 to 7 membered heterocyclic ring, wherein said heterocyclic ring may be optionally substituted with C₁₋₃ alkyl or halogen; and
[symbol] is a single or double bond or a cyclopropyl ring;
provided that compounds of formula (1) having a linear C₁₋₅ acyl group, a valeroyl group or a cyclopropylcarbonyl group, as —XYZ are excluded;
or a pharmaceutically acceptable salt thereof.

In an alternative embodiment of the compound of the first aspect, the variables of the formula (1) are as follows:
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C₁₋₁₂ alkylthio, aryl, heterocyclyl, heteroaryl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, cycloalkyl, cycloalkoxy, aryl, heterocyclyl or heteroaryl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂;
R² is selected from hydrogen, C₁₋₃ alkyl, C₃₋₅ cycloalkyl, oxetane, C₁₋₃ haloalkyl, C₁₋₃ alkylthio, halogen, cyano, COOH and CONH₂;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or
R^{a} and R^{b}, together with the adjacent nitrogen atom, form a 3 to 7 membered heterocyclic ring, wherein said heterocyclic ring may be optionally substituted with C₁₋₃ alkyl or halogen; and
[symbol] is a single or double bond or a cyclopropyl ring;
provided that
(i) if [symbol] is a double bond, R² is hydrogen, R⁶ is methyl, R^{a} and R^{b} are both ethyl, and X is CO, then Y—Z is not methyl, ethyl, n-propyl, n-butyl or cyclopropyl;
(ii) if [symbol] is a double bond, R² is hydrogen, R⁶ is methyl, one of R^{a} and R^{b} is ethyl and the other is hydrogen, and X is CO, then Y—Z is not methyl;
(iii) if [symbol] is a double bond, R² is hydrogen, R⁶ is methyl, one of R^{a} and R^{b} is methyl and the other is isopropyl, and X is CO, then Y—Z is not ethyl or cyclopropyl;
(iv) if [symbol] is a double bond, R² is hydrogen, R⁶ is ethyl, R^{a} and R^{b} are both ethyl, and X is CO, then Y—Z is not ethyl; and
(v) if [symbol] is a double bond, R² is hydrogen, R⁶ is allyl, R^{a} and R^{b} are both ethyl, and X is CO, then Y—Z is not ethyl or cyclopropyl.

(Said provisos (i) to (v) excluding the previously described compounds, notably ALD-52, 1P-LSD, 1P-ETH-LAD, 1P-MIPLA, 1P-AL-LAD, 1B-LSD, 1 cP-LSD, 1 cP-AL-LAD, 1 cP-MIPLA and 1V-LSD).

The compounds of formula (1) of both alternatives of the first aspect are hereinafter shortly referred to as “compounds of the invention”.

As a second aspect, the present invention provides (I) a pharmaceutical composition comprising a compound of the invention as described in the above-mentioned first aspect, or a pharmaceutically acceptable salt thereof and a pharmaceutically acceptable carrier, and (II) a medicament comprising a compound of the invention as described in the above-mentioned first aspect, or a pharmaceutically acceptable salt thereof.

As a third aspect, the present invention provides a compound of formula (1)

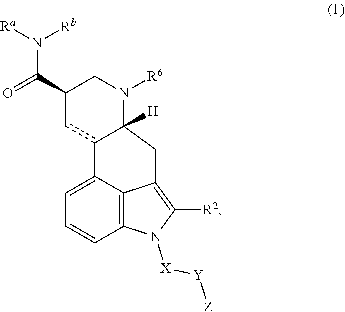

wherein
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C₁₋₁₂ alkylthio, aryl, heterocyclyl, heteroaryl, metallocenyl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, alkylthio, cycloalkyl, cycloalkoxy, aryl, heterocyclyl, heteroaryl or metallocenyl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂;
R² is selected from hydrogen, C₁₋₃ alkyl, C₃₋₅ cycloalkyl, oxetane, C₁₋₃ haloalkyl, C₁₋₃ alkylthio, halogen, cyano, COOH and CONH₂;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or
R^{a} and R^{b} together with the adjacent nitrogen atom form a 3 to 7 membered heterocyclic ring, wherein said heterocyclic ring may be optionally substituted with C₁₋₃ alkyl or halogen; and
[symbol] is a single or double bond or a cyclopropyl ring;
provided that if [symbol] is a double bond, R² is hydrogen, R⁶ is methyl, R^{a} and R^{b} are both ethyl, and X is CO, then Y—Z is not methyl,
or a pharmaceutically acceptable salt thereof, for use in treating diseases, requiring modulated or reduced hallucinogenic side effects as compared to LSD and 1-acetyl-LSD, in a subject. In particular, the compound of formula (1) is for use in treating anxiety in a subject, for use in treating depression in a subject, for use in treating migraine headache in a subject, for use in treating cluster headache in a subject, for use in treating glaucoma in a subject, for use in treating macular degeneration in a subject, for use in treating rheumatoid arthritis in a subject and for use in treating Alzheimer's disease in a subject.

As a fourth aspect, the present invention provides a method for treating diseases requiring a modulated or reduced hallucinogenic side effects as compared to LSD and 1-acetyl-LSD, said method comprising administering a compound of formula (1) as described in the above-mentioned third aspect, or a pharmaceutically acceptable salt thereof, to a subject (or patient) in need of such treatment.

As a further general aspect the present invention provides a method for producing compound of the invention as described in the above-mentioned first aspect, or a pharmaceutically acceptable salt thereof, said method comprises converting a suitable precursor compound into a compound having the formula (1), e.g. as described in the General Methods A-F below. As a particular fifth aspect, the present invention provides a method for producing a compound of formula (1)

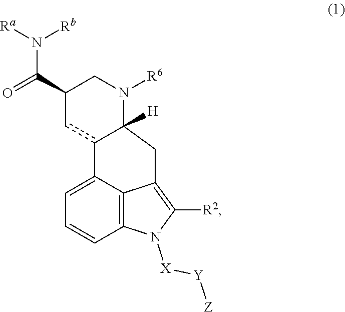

wherein
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C_{1-1 2} alkylthio, aryl, heterocyclyl, heteroaryl, metallocenyl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, cycloalkyl, alkylthio, cycloalkoxy, aryl, heterocyclyl, heteroaryl or metallocenyl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂; or —XYZ together represent hydrogen;
R² is Br;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or
R^{a} and R^{b} together with the adjacent nitrogen atom form a 3 to 7 membered heterocyclic ring, wherein said heterocyclic ring may be optionally substituted with C₁₋₃ alkyl or halogen; and
[symbol] is a single or double bond or a cyclopropyl ring; said method comprises saponifying bromocriptine followed by amide-formation as shown in the following reaction scheme:

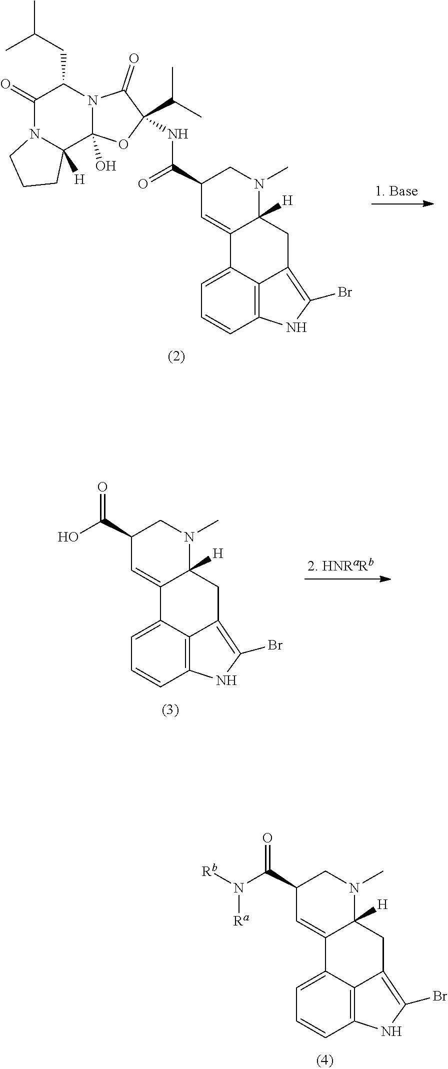

wherein the variables are as defined above.

As a sixth aspect, the present invention provides intermediate compounds of the formula (4)

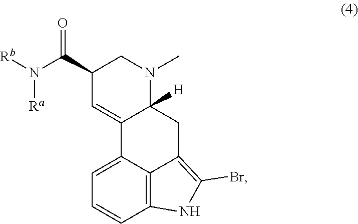

wherein R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or R^{a} and R^{b} together with the adjacent nitrogen atom form a 3 to 7 membered heterocyclyl ring, wherein said heterocyclyl ring may be optionally substituted with C₁₋₃ alkyl or halogen,
or a salt thereof.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other features of the invention may become apparent from the following description which is given by way of example only.

### SHORT DESCRIPTION OF THE FIGURES

FIG. 1: LSD plasma concentration. Data are the mean±SEM in ng/ml (n=3 mice/group). Mice were injected i.p. with vehicle or drug (1 mg/kg) and then plasma was collected 30 min later.

FIG. 2: Normalized LSD plasma concentration. Data are shown as ng/ml LSD per μmol/kg of injected drug.

FIG. 3: Effect of test substances on the head-twitch response (HTR) in male C₅₇BL/6J mice. Data are the mean±SEM of HTR counts. Mice were injected i.p. with vehicle or drug (1 mg/kg) and then HTR behavior was assessed continuously for 30 min using a head-mounted magnet and a magnetometer coil.35 *p<0.05 vs vehicle, Dunnett's test. In this figure ALD-52 (1-acetyl-LSD) is shown as a comparison (data from Adam L. Halberstadt, et al. (2019) DOI: 10.1016/j.neuropharm.2019.107856), and with an average HTR count of 16.6 at the same 1 mg/kg dosage and under the same assay conditions, ALD-52 produces less HTR counts in 30 min than the compounds of the present invention.

FIG. 4: Normalized HTR counts. The mean±SEM of HTR counts for each drug was divided by the dose administered (in μmol/kg).

### DETAILED DESCRIPTION OF THE INVENTION

### I. Definitions

As used herein “(s)” following a noun means the plural and/or singular forms of the noun.

As used herein the term “and/or” means “and” or “or” or both.

As used herein, the term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly 10 disclosed herein are hereby expressly disclosed. These are only examples of what is specifically intended and all possible combinations of numerical values between the lowest value and the highest value enumerated are to be considered to be expressly stated in this application in a similar manner.

The term “therapeutically effective amount”, as used herein, means the amount of an active compound, or a material, composition, composition, or dosage form comprising an active compound, which is effective for producing some desired therapeutic or prophylactic effect, commensurate with a reasonable benefit/risk ratio. Therapeutically effective amounts can be determined using routine optimisation techniques well known in the art.

The term “pharmaceutically acceptable” as used herein in conjunction with the terms “salt”, “prodrug”, or “carrier” refers to compounds of the invention, ingredients, materials and the like, which are within the scope of sound medical judgment, suitable for use in contact with the tissues of a human without excessive toxicity, irritation, allergic response, or other problem or complication, commensurate with a reasonable benefit/risk ratio. Each carrier must also be “acceptable” in the sense of being compatible with the other ingredients of the composition.

The term “treatment”, and related terms, such as “treating” and “treat” as used herein, in the context of treating a condition, relates generally to treatment of a human, in which some desired therapeutic effect is achieved. The therapeutic effect may, for example, be the inhibition of progress of the condition, including a reduction in the rate of progress; a halt in the rate of progress of the condition; amelioration of the condition; and cure of the condition.

Treatment as a prophylactic measure (i.e., prophylaxis) is also included. Treatment also includes combination treatments and therapies, in which two or more treatments or therapies are used, for example, sequentially or simultaneously, in combination. For example, a therapeutically effective amount of a compound of the invention could be combined with or used in conjunction with a known compound having therapeutic effects on the same condition but utilising a different mechanism of action.

The term “psychedelic” as used herein means having similar effects to the effects of LSD when administered to a subject.

The term “lysergamide” as used herein means chemical compounds having a chemical structure derived from that of lysergic acid amide, 9,10-didehydro-6-methyl-ergoline-8-carboxamide.

The term “alkyl” means a saturated hydrocarbon radical containing normal, secondary, or tertiary carbon atoms. Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

The term “alkenyl” means a hydrocarbon radical containing normal, secondary, tertiary or 25 cyclic carbon atoms with at least one double bond. Examples of suitable alkenyl groups include, but are not limited to, ethylene or vinyl (—CH═CH₂), allyl (—CH₂CH═CH₂), cyclopentenyl (—C₅H₇), and 5-hexenyl (—CH₂CH₂CH₂CH₂CH═CH₂).

The terms “alkylene” and “alkenylene” refer to divalent residues based on the above-mentioned alkyl and alkenyl groups.

The term “alkynyl” means a hydrocarbon radical containing normal, secondary, tertiary or cyclic carbon atoms with at least one triple bond. Examples of suitable alkynyl groups include, but are not limited to, acetylenic (—C≡CH), propargyl (—CH2C≡CH), and the like.

The term “cycloalkyl” means a hydrocarbon radical containing a saturated ring having 3 to 7 carbon atoms as a monocycle, 7 to 12 carbon atoms as a bicycle, and up to about 20 carbon atoms as a polycycle. Monocyclic carbocycles have 3 to 7 ring atoms, still more typically 5 or 6 ring atoms. Bicyclic carbocycles have 7 to 12 ring atoms, e.g., arranged as a bicyclo[4,5], [5,5], [5,6] or [6,6] system, or 9 or 10 ring atoms arranged as a bicyclo[5,6] or [6,6] system, or spiro-fused rings.

The terms “cycloalkenyl” and “cycloalkynyl” have analogous meanings.

The term “aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 12 carbon atoms. Typical aryl groups include, but are not limited to, radicals derived from benzene (e.g., phenyl), substituted benzene, naphthalene, anthracene, biphenyl, and the like.

The term “alkoxy” means a group having the formula —O-alkyl, in which an alkyl group, as defined above, is attached to the parent molecule via an oxygen atom. Examples of suitable alkoxy groups include, but are not limited to, methoxy (—OCH₃ or —OMe), ethoxy (—OCH₂CH₃ or —OEt), t-butoxy (—OC(CH₃)₃ or -OtBu) and the like.

The term “alkylthio” means a group having the formula —S-alkyl, in which an alkyl group, as defined above, is attached to the parent molecule via a sulfur atom.

Examples of suitable alkylthio groups include, but are not limited to, methylthio (—S—CH₃ or —SMe), ethylthio (—SCH₂CH₃ or —SEt), t-butylthio (—SC(CH₃)₃ or -StBu) and the like.

The term “halo” as used herein means iodo, bromo, chloro, or fluoro.

The term “haloalkyl” means an alkyl group, as defined above, in which one or more hydrogen atoms of the alkyl group is replaced with a halo group. Examples of suitable haloalkyl groups include, but are not limited to, —(CH₂)ₙF, —CF₃, —CHF₂, —CFH₂, —CH₂CF₃, and the like.

The terms “haloalkenyl” and “haloalkynyl” have analogous meanings.

The term “optionally substituted” in reference to a particular moiety of the compound of the 30 invention (e.g., an optionally substituted aryl group) refers to a moiety having 0, 1, 2, or more substituents.

The term “substituted” in reference to alkyl, alkenyl, alkynyl, halogen, alkoxy, aryl, cycloalkyl, heteroalkyl, heterocyclyl, or heteroaryl means alkyl, alkenyl, alkynyl, halogen, alkoxy, aryl, cycloalkyl, heteroalkyl, heterocyclyl, or heteroaryl respectively, in which one or more hydrogen atoms are each independently replaced with a non-hydrogen substituent. Typical substituents include, but are not limited to, —X, —R, ═O, —OR, —SR, —NR₂, —N⁺R₃, ═NR, —CX3, —CN, —OCN, —SCN, —N═C═O, —NCS, —NO, —NO2, ═N₂, —N₃, —NHC(═O)R, —NHS(═O)₂R, —C(═O)R, —C(═O)NRR, —S(═O)₂OH, —S(═O)₂R, —OS(═O)₂OR, —S(═O)₂NR, —S(═O)R, —OP(═O)(OR)₂, —P(═O)(OR)₂, —P(═O)(OH)₂, —C(═O)R, —C(═O)OR, —C(═O)X, —C(S)R, —C(O)OR, —C(S)OR, —C(O)SR, —C(S)SR, —C(O)NRR, —C(S)NRR, or —C(═NR)NRR, where each X is independently halo; and each R is independently H, C₁-C₁₀ alkyl, C₆-C₁₂ aryl, C₆-C₁₂ fluoroaryl, C₆-C₁₂ aryl C₁-C₈ alkyl, C₃-C₁₂ cycloalkyl, bridged C₆-C₁₂ cycloalkyl, halo or perhalo substituted C₃-C₁₂ cycloalkyl, a C₃-C₁₂ heterocycle, or a protecting group (PG). When the number of carbon atoms is designated for a substituted group, the number of carbon atoms refers to the group, not the substituent (unless otherwise indicated). For example, a C₁₋₄ substituted alkyl refers to a C₁₋₄ alkyl, which can be substituted with groups having more than the, e.g., 4 carbon atoms.

The term “prodrug” as used herein refers to any compound that when administered to a biological system generates the active ingredient, as a result of spontaneous chemical reaction(s), enzyme catalysed chemical reaction(s), photolysis, and/or metabolic chemical reaction(s). A prodrug is thus a covalently modified analogue or latent form of a therapeutically active compound.

Those skilled in the art will recognise that substituents and other moieties of the compounds of Formula I should be selected in order to provide a compound which is sufficiently stable to provide a pharmaceutically useful compound which can be formulated into an acceptably stable pharmaceutical composition. Compounds of the invention which have such stability are contemplated as falling within the scope of the present invention.

The term “heteroalkyl” refers to an alkyl group where one or more carbon atoms have been replaced with a heteroatom, such as, O, N, or S. For example, if the carbon atom of the alkyl group which is attached to the parent molecule is replaced with a heteroatom (e.g., O, N, or S) the resulting heteroalkyl groups are, respectively, an alkoxy group (e.g., —OCH₃, etc.), an amine (e.g., —NHCH₃, —N(CH₃)₂, etc.), or a thioalkyl group (e.g., —SCH₃). If a non-terminal carbon atom of the alkyl group which is not attached to the parent molecule is replaced with a heteroatom (e.g., O, N, or S) the resulting heteroalkyl groups are, respectively, an alkyl ether (e.g., —CH₂CH₂—O—CH₃, etc.), an alkyl amine (e.g., —CH₂NHCH₃, —CH₂N(CH₃)₂, etc.), or a thioalkyl ether (e.g., —CH₂—S—CH₃). If a terminal carbon atom of the alkyl group is replaced with a heteroatom (e.g., O, N, or S), the resulting heteroalkyl groups are, respectively, a hydroxyalkyl group (e.g., —CH₂CH₂—OH), an aminoalkyl group (e.g., —CH₂NH₂), or an alkyl thiol group (e.g., —CH₂CH₂—SH). A heteroalkyl group can have, for example, 1 to 20 carbon atoms, 1 to 10 carbon atoms, or 1 to 6 carbon atoms. A C₁₋₆ heteroalkyl group means a heteroalkyl group having 1 to 6 carbon atoms.

The terms “heterocycle” or “heterocyclyl” as used herein include by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. 15 Chem. Soc. (1960) 82:5566. The terms “heterocycle” and “heterocyclyl” include saturated rings (i.e., heterocycloalkyls), partially unsaturated rings, and aromatic rings (i.e., heteroaromatic rings). The terms “heterocycle” and “heterocyclyl” also include substituted heterocyclyls include, for example, heterocyclic rings substituted with any of the substituents disclosed herein including carbonyl groups. Examples of substituted heterocycles include, but are not limited to, 5-(t-butyl)-furan-2-yl and 2,4-dimethylazetidinyl.

The term “heterocycle” or “heterocyclyl” employed alone or in combination with other terms means, unless otherwise stated, a saturated or unsaturated non-aromatic monocyclic heterocyclyl ring or a bicyclic heterocyclyl ring. Monocyclic heterocyclyl rings include monovalent 3-, 4-, 5-, 6-, or 7-membered rings containing one or more heteroatoms independently selected from the group consisting of oxygen, nitrogen, sulfur and selenium in the ring. Monocyclic heterocyclyl groups are connected to the parent molecular moiety through any available carbon atom or nitrogen atom within the ring. Examples of monocyclic heterocyclyl groups include, but are not limited to, azetidinyl, azepanyl, aziridinyl, diazepanyl, 1,3-dioxanyl, 1,3-dioxolanyl, 1,2-dithiolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazetidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, tetrazolyl, thiadiazolinyl, thiadiazolidinyl, thiazolinyl, thiazolidinyl, thiopyranyl, triazolyl, and trithianyl. Bicyclic heterocyclyl rings include monovalent monocyclic heterocyclyl rings fused to phenyl rings, cycloalkyl rings, or other monocyclic heterocyclyl rings. Bicyclic heterocyclyl groups are connected to the parent molecular moiety through any available carbon atom or nitrogen atom within the rings. Examples of bicyclic heterocyclyl groups include, but are not limited to, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, and 1,2,3,4-tetrahydroquinolinyl. In some embodiments the heterocyclyl is monocyclic.

“Heteroaryl” refers to an aromatic heterocyclyl. Non-limiting examples of suitable heteroatoms which can be included in the aromatic ring include oxygen, sulphur, selenium and nitrogen. Non-limiting examples of heteroaryl rings include pyridinyl, pyrrolyl, oxazolyl, indolyl, isoindolyl, purinyl, furanyl, thienyl, selenophenyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, etc.

By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

The term “metallocene” or “metallocenyl” refers to a ring system where two cyclopentadienyl, phenyl, cycloheptatrienyl or cyclooctatetraenidinyl rings are joined by haptic covalent bonds to a bridging metal atom. Non-limiting examples of metallocenyl rings include ferrocene, titanocene, chromocene and vanadocene.

In a preferred embodiment, the metallocenyl ring is ferrocene.

The term “halogenating agent” includes, but are not limited to, phosphorus oxychloride, thionyl chloride, oxalyl chloride, phosphorus trichloride, phosphorus pentachloride, and diphosgene.

The term “halogenated solvent” includes, but are not limited to, chloroform, dichloromethane, and carbon tetrachloride.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution but differ with regard to the arrangement of the atoms or groups in space.

“Diastereomer” refers to a stereoisomer with two or more centres of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography. “Enantiomers” refer to two stereoisomers of a compound which are non-superimposable mirror images of one another.

### II. The Compounds of the Invention

The first aspect of the invention provides substituted ergoline compounds that have a range of therapeutic activities including antidepressant, anti-anxiety, anti-inflammatory, and anti-cephalagic. The compounds of the invention also have psychotropic effects and can be used for psychiatric therapy.

In particular, the first aspect of the invention provides a compound of formula (1),

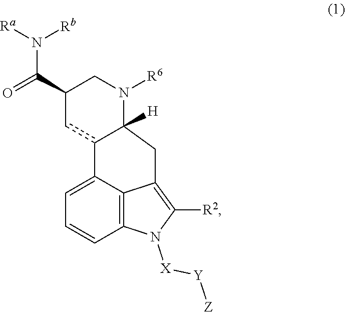

wherein all the variables are as defined hereinbefore.

With the provisos in the definition of the first aspect above it is intended to exclude the particular prior art compounds of formula (1) having a C₁₋₅ acyl group, notably a linear C₁₋₅ acyl group, a valeroyl group or a cyclopropylcarbonyl group, as —XYZ. The following preferred embodiments of the compound of formula (1) relate to any of the aspects relating to the compound of formula (1) above, as appropriate.

In a particular embodiment of the compound of formula (1), R^{a} and R^{b} are both ethyl. In another particular embodiment, R^{a} and R^{b} are selected such that one group is methyl and the other is isopropyl. In a further particular embodiment, R^{a} and R^{b} together with the adjacent nitrogen form a substituted 4 membered heterocyclic ring, such as a 2,4-dimethylazetidine ring where the two methyl groups are oriented with (S,S) stereochemistry.

In a particular embodiment of the compound of formula (1), R⁶ is selected from methyl, ethyl, allyl or methallyl.

In a particular embodiment of the compound of formula (1), R² is selected from methyl, fluoro, bromo, trifluoromethyl and cyclopropyl.

In a particular embodiment of the compound of formula (1), X is CO. In another particular embodiment, X is SO₂.

In a particular embodiment of the compound of formula (1), Y is a chemical bond.

In another particular embodiment, Y is O. In a further particular embodiment, Y is selected from methyl, ethyl and propyl. In still a further particular embodiment, Y is O—CH(CH₃).

In a particular embodiment of the compound of formula (1), Z is selected from ethyl, propyl, isopropyl, butyl, isobutyl, t-butyl, pentyl, 4-methylpentyl, 1,2,2-trimethylpropyl, hexyl and undecyl. In another particular embodiment, Z is selected from cyclopropyl, 2,2,3,3-tetramethylcyclopropyl, cyclobutyl, cyclopentyl and cyclohexyl. In a further particular embodiment, Z is selected from bicyclo[1.1.1]pentyl, quinuclidinyl and adamantyl. In still a further particular embodiment, Z is selected from oxetan-3-yl, tetrahydrofuran-2-yl, tetrahydropyran-4-yl and morpholin-4-yl. In still a further particular embodiment, Z is selected from phenyl, 2-methylphenyl, 4-methylphenyl, furan-2-yl, 4-methylfuran-2-yl, 4-(t-butyl)furan-2-yl, furan-3-yl, thiophen-2-yl, thiophen-3-yl, pyridin-3-yl, pyridin-4-yl, 5-bromo-thiophen-2-yl or selenophen-3-yl. In still a further embodiment, Z is COOH or COOCH₃. In still a further embodiment, Z is O—CO-isopropyl. In still a further embodiment, Z is a leaving group such as halogen, which may be further reacted to form additional compounds of the invention, or Z is a protecting group such as tert-butoxycarbonyl, which may be further reacted to form additional compounds of the invention.

In a particular embodiment of the compound of formula (1), [symbol] is a double bond.

In another particular embodiment, [symbol] is cyclopropyl. In a further particular embodiment, [symbol] is a single bond.

The following specific generic embodiments (1) to (14) of the compound of formula (1) of the invention are provided:
(1) compounds in which R^{a} and R^{b} are both ethyl; R⁶ is methyl; R² is hydrogen; X is SO₂; Y is a chemical bond; Z is selected from cyclopropyl and 4-methylphenyl; and [symbol] is a double bond;
(2) compounds in which R^{a} and R^{b} are both ethyl; R⁶ is methyl; R² is hydrogen; X is CO; Y is O; Z is selected from ethyl and t-butyl; and [symbol] is a double bond;
(3) compounds in which R^{a} and R^{b} are both ethyl; R⁶ is methyl; R² is hydrogen; X is CO; Y is O—CH₂; Z is phenyl; and [symbol] is a double bond;
(4) compounds in which R^{a} and R^{b} are both ethyl; R⁶ is methyl; R² is hydrogen; X is CO; Y is a chemical bond; Z is selected from isopropyl, butyl, isobutyl, t-butyl, pentyl, 2,3,3-trimethylpropyl and undecanyl; and [symbol] is a double bond;
(5) compounds in which R^{a} and R^{b} are both ethyl; R⁶ is methyl; R² is hydrogen; X is CO; Y is a chemical bond; Z is selected from 2,2,3,3-tetramethylcyclopropyl, cyclobutyl, cyclopentyl and cyclohexyl; and [symbol] is a double bond;
(6) compounds in which R^{a} and R^{b} are both ethyl; R⁶ is methyl; R² is hydrogen; X is CO; Y is a chemical bond; Z is selected from bicyclo[1.1.1] pent-1-yl and adamant-1-yl; and [symbol] is a double bond;
(7) compounds in which R^{a} and R^{b} are both ethyl; R⁶ is methyl; R² is hydrogen; X is CO; Y is a chemical bond; Z is selected from oxetan-3-yl, tetrahydrofuran-2-yl and tetrahydropyran-4-yl; and [symbol] is a double bond;
(8) compounds in which R^{a} and R^{b} are both ethyl; R⁶ is methyl; R² is hydrogen; X is CO; Y is a chemical bond; Z is selected from phenyl, 2-methylphenyl, furan-2-yl, 4-methylfuran-2-yl, 4-(t-butyl)furan-2-yl, furan-3-yl, thiophen-2-yl, thiophen-3-yl, pyridin-3-yl, pyridin-4-yl and morpholin-4-yl; and [symbol] is a double bond;
(9) compounds in which R^{a} and R^{b} are both ethyl; R⁶ is methyl; R² is hydrogen; X is CO; Y is methyl; Z is selected from phenyl, cyclohexyl, tetrahydropyran-4-yl and methoxy; and [symbol] is a double bond;
(10) compounds in which R^{a} and R^{b} are both ethyl; R⁶ is methyl; R² is hydrogen; X is CO; Y is ethyl; Z is selected from phenyl, cyclopentyl, COOH and COOCH₃; and [symbol] is a double bond;
(11) compounds in which R^{a} and R^{b} are both ethyl; R⁶ is methyl; R² is hydrogen; X is CO; Y is propyl; Z is morpholin-4-yl; and [symbol] is a double bond.
(12) compounds in which R^{a} and R^{b} are both ethyl; R⁶ is methyl; R² is hydrogen; X is CO; Y is O—CH(CH₃); Z is O—CO-isopropyl; and [symbol] is a double bond;
(13) compounds in which, R^{a} and R^{b} are both ethyl; R⁶ is methyl; R² is bromo; X is CO; Y is a chemical bond; Z is selected from ethyl, cyclopropyl and furan-2-yl; and [symbol] is a double bond;
(14) compounds in which R^{a} and R^{b} are both ethyl; R⁶ is methyl; R² is bromo; X is CO; Y is ethyl; Z is selected from phenyl and cyclopentyl; and [symbol] is a double bond; and
(15) compounds in which R^{a} and R^{b} are both ethyl; R⁶ is methyl; R² is hydrogen; X is CO; Y is a chemical bond; Z is butyl-3-one, selenophen-3-yl or ferrocenyl; and [symbol] is a double bond.

In a particular specific embodiment, the compound of the invention is selected from the following (a) to (mmm):

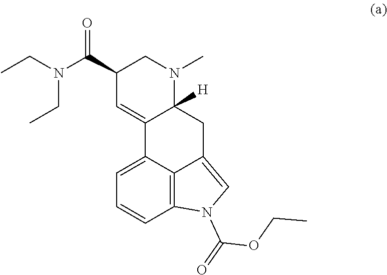 [SMILES: CCOC(=O)n1cc2c3c(cccc31)C1=C[C@@H](C(=O)N(CC)CC)CN(C)[C@@H]1C2]

SYN-L-003, 1-ethylcarbamoyl-lysergic acid diethylamide, or a pharmaceutically acceptable salt thereof;

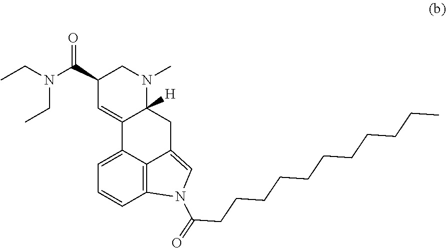 [SMILES: CCCCCCCCCCCC(=O)n1cc2c3c(cccc31)C1=C[C@@H](C(=O)N(CC)CC)CN(C)[C@@H]1C2]

SYN-L-004, 1-dodecanoyl-lysergic acid diethylamide, or a pharmaceutically acceptable salt thereof;

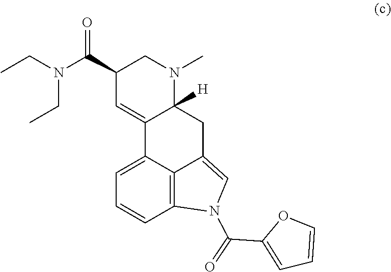 [SMILES: CCN(CC)C(=O)[C@@H]1C=C2c3cccc4c3c(cn4C(=O)c3ccco3)C[C@H]2N(C)C1]

SYN-L-005, 1-(2-furoyl)-lysergic acid diethylamide, or a pharmaceutically acceptable salt thereof;

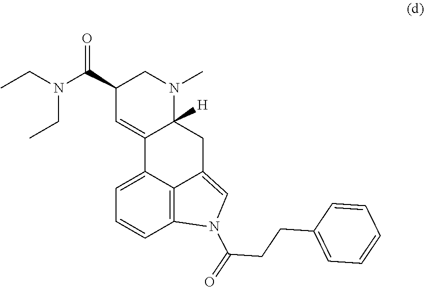 [SMILES: CCN(CC)C(=O)[C@@H]1C=C2c3cccc4c3c(cn4C(=O)CCc3ccccc3)C[C@H]2N(C)C1]

SYN-L-006, 1-(3-phenylpropanoyl)-lysergic acid diethylamide, or a pharmaceutically acceptable salt thereof;

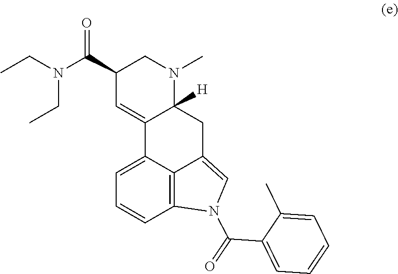 [SMILES: CCN(CC)C(=O)[C@@H]1C=C2c3cccc4c3c(cn4C(=O)c3ccccc3C)C[C@H]2N(C)C1]

SYN-L-008, 1-(o-toluoyl)-lysergic acid diethylamide, or a pharmaceutically acceptable salt thereof;

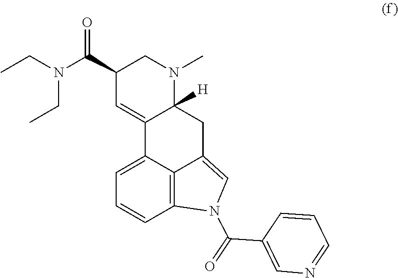 [SMILES: CCN(CC)C(=O)[C@@H]1C=C2c3cccc4c3c(cn4C(=O)c3cccnc3)C[C@H]2N(C)C1]

SYN-L-010, 1-nicotinoyl-lysergic acid diethylamide, or a pharmaceutically acceptable salt thereof;

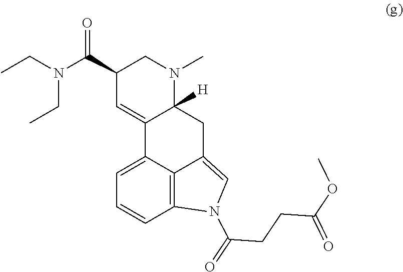 [SMILES: CCN(CC)C(=O)[C@@H]1C=C2c3cccc4c3c(cn4C(=O)CCC(=O)OC)C[C@H]2N(C)C1]

SYN-L-012, 1-methylsuccinyl-lysergic acid diethylamide, or a pharmaceutically acceptable salt thereof;

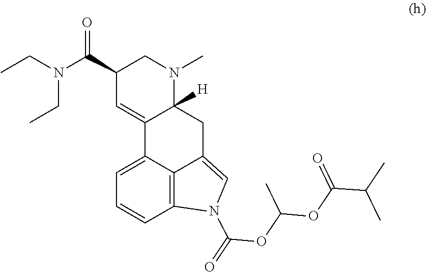 [SMILES: CCN(CC)C(=O)[C@@H]1C=C2c3cccc4c3c(cn4C(=O)OC(C)OC(=O)C(C)C)C[C@H]2N(C)C1]

SYN-L-013, 1-enacarbil-lysergic acid diethylamide, or a pharmaceutically acceptable salt thereof;

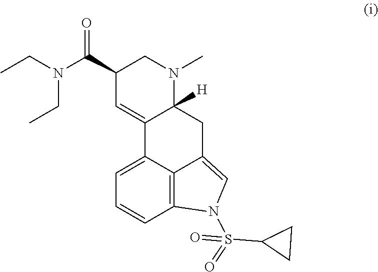 [SMILES: CCN(CC)C(=O)[C@@H]1C=C2c3cccc4c3c(cn4S(=O)(=O)C3CC3)C[C@H]2N(C)C1]

SYN-L-014, 1-cyclopropanesulfonyl-lysergic acid diethylamide, or a pharmaceutically acceptable salt thereof;

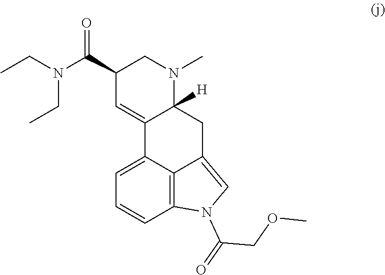 [SMILES: CCN(CC)C(=O)[C@@H]1C=C2c3cccc4c3c(cn4C(=O)COC)C[C@H]2N(C)C1]

SYN-L-015, 1-methoxyacetyl-lysergic acid diethylamide, or a pharmaceutically acceptable salt thereof;

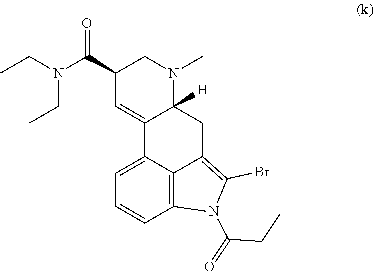 [SMILES: CCC(=O)n1c(Br)c2c3c(cccc31)C1=C[C@@H](C(=O)N(CC)CC)CN(C)[C@@H]1C2]

SYN-L-017, 1-propionyl-2-bromo-lysergic acid diethylamide, or a pharmaceutically acceptable salt thereof;

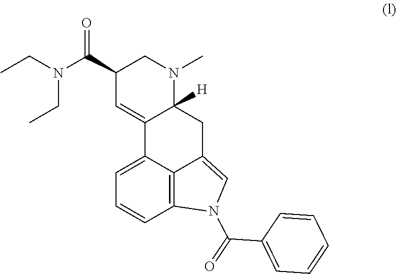 [SMILES: CCN(CC)C(=O)[C@@H]1C=C2c3cccc4c3c(cn4C(=O)c3ccccc3)C[C@H]2N(C)C1]

SYN-L-018, 1-benzoyl-lysergic acid diethylamide, or a pharmaceutically acceptable salt thereof;

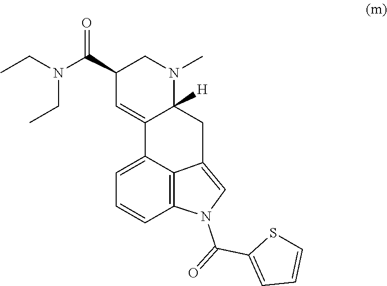 [SMILES: CCN(CC)C(=O)[C@@H]1C=C2c3cccc4c3c(cn4C(=O)c3cccs3)C[C@H]2N(C)C1]

SYN-L-021, 1-(2-thiophenecarbonyl)-lysergic acid diethylamide, or a pharmaceutically acceptable salt thereof;

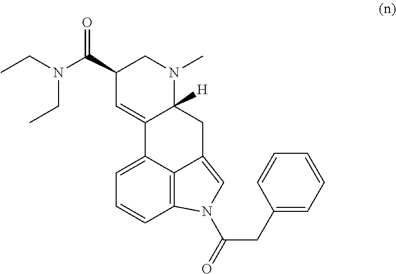 [SMILES: CCN(CC)C(=O)[C@@H]1C=C2c3cccc4c3c(cn4C(=O)Cc3ccccc3)C[C@H]2N(C)C1]

SYN-L-022, 1-phenylacetyl-lysergic acid diethylamide, or a pharmaceutically

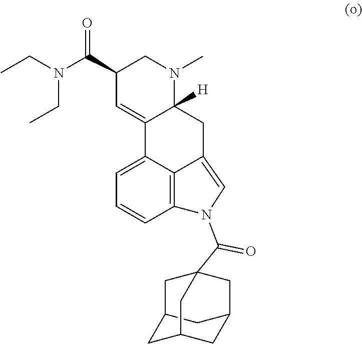 [SMILES: CCN(CC)C(=O)[C@@H]1C=C2c3cccc4c3c(cn4C(=O)C34CC5CC(CC(C5)C3)C4)C[C@H]2N(C)C1]

SYN-L-023, 1-(1-adamantanecarbonyl)-lysergic acid diethylamide, or a pharmaceutically acceptable salt thereof;

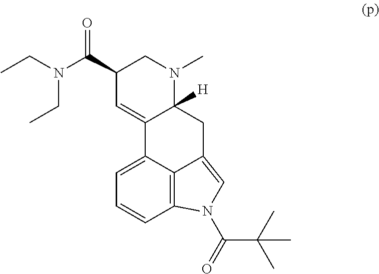 [SMILES: CCN(CC)C(=O)[C@@H]1C=C2c3cccc4c3c(cn4C(=O)C(C)(C)C)C[C@H]2N(C)C1]

SYN-L-024, 1-pivaloyl-lysergic acid diethylamide, or a pharmaceutically acceptable salt thereof.

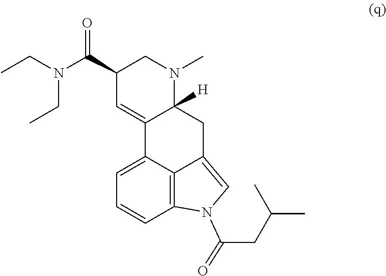 [SMILES: CCN(CC)C(=O)[C@@H]1C=C2c3cccc4c3c(cn4C(=O)CC(C)C)C[C@H]2N(C)C1]

SYN-L-026, 1-isovaleryl-lysergic acid diethylamide, or a pharmaceutically acceptable salt thereof;

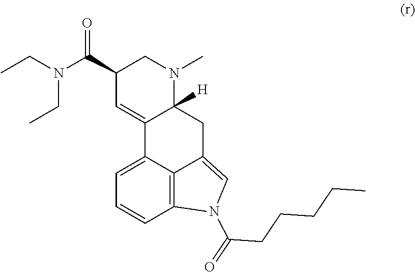 [SMILES: CCCCCC(=O)n1cc2c3c(cccc31)C1=C[C@@H](C(=O)N(CC)CC)CN(C)[C@@H]1C2]

SYN-L-027, 1-hexanoyl-lysergic acid diethylamide, or a pharmaceutically acceptable salt thereof;

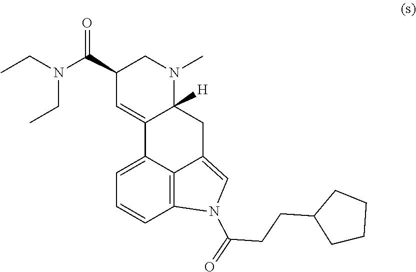 [SMILES: CCN(CC)C(=O)[C@@H]1C=C2c3cccc4c3c(cn4C(=O)CCC3CCCC3)C[C@H]2N(C)C1]

SYN-L-028, 1-cyclopentanepropanoyl-lysergic acid diethylamide, or a pharmaceutically acceptable salt thereof;

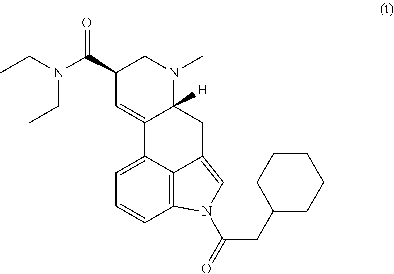 [SMILES: CCN(CC)C(=O)[C@@H]1C=C2c3cccc4c3c(cn4C(=O)CC3CCCCC3)C[C@H]2N(C)C1]

SYN-L-029, 1-cyclohexanecarbonyl-lysergic acid diethylamide, or a pharmaceutically acceptable salt thereof;

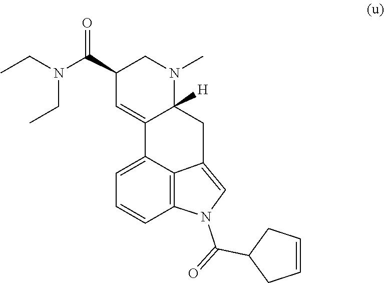 [SMILES: CCN(CC)C(=O)[C@@H]1C=C2c3cccc4c3c(cn4C(=O)C3CC=CC3)C[C@H]2N(C)C1]

SYN-L-030, 1-cyclopentanecarbonyl-lysergic acid diethylamide, or a pharmaceutically acceptable salt thereof;

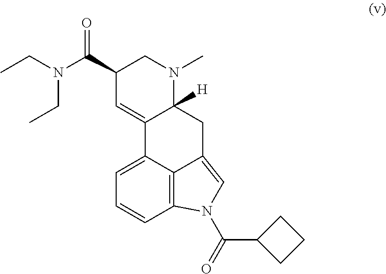 [SMILES: CCN(CC)C(=O)[C@@H]1C=C2c3cccc4c3c(cn4C(=O)C3CCC3)C[C@H]2N(C)C1]

SYN-L-031, 1-cyclobutanecarbonyl-lysergic acid diethylamide, or a pharmaceutically acceptable salt thereof.

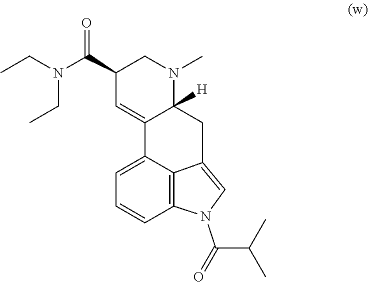 [SMILES: CCN(CC)C(=O)[C@@H]1C=C2c3cccc4c3c(cn4C(=O)C(C)C)C[C@H]2N(C)C1]

SYN-L-032, 1-isobutyryl-lysergic acid diethylamide, or a pharmaceutically acceptable salt thereof;

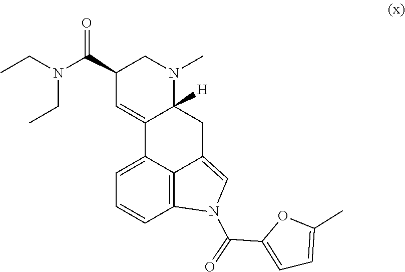 [SMILES: CCN(CC)C(=O)[C@@H]1C=C2c3cccc4c3c(cn4C(=O)c3ccc(C)o3)C[C@H]2N(C)C1]

SYN-L-034, 1-(5-methyl-2-furoyl)-lysergic acid diethylamide, or a pharmaceutically acceptable salt thereof;

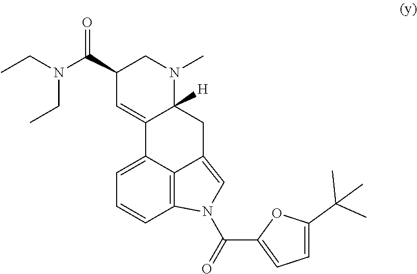 [SMILES: CCN(CC)C(=O)[C@@H]1C=C2c3cccc4c3c(cn4C(=O)c3ccc(C(C)(C)C)o3)C[C@H]2N(C)C1]

SYN-L-035, 1-(5-tert-butyl-2-furoyl)-lysergic acid diethylamide, or a pharmaceutically acceptable salt thereof;

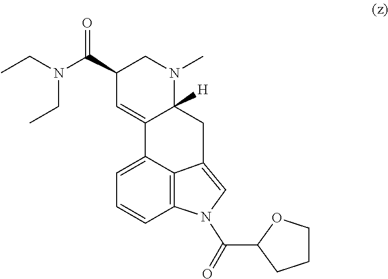 [SMILES: CCN(CC)C(=O)[C@@H]1C=C2c3cccc4c3c(cn4C(=O)C3CCCO3)C[C@H]2N(C)C1]

SYN-L-036, 1-(tetrahydro-2-furoyl)-lysergic acid diethylamide, or a pharmaceutically acceptable salt thereof;

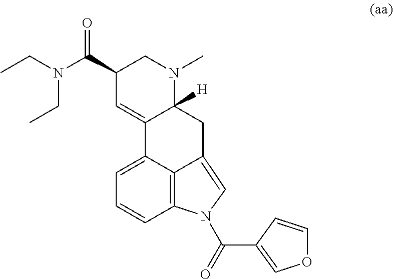 [SMILES: CCN(CC)C(=O)[C@@H]1C=C2c3cccc4c3c(cn4C(=O)c3ccoc3)C[C@H]2N(C)C1]

SYN-L-037, 1-(3-furoyl)-lysergic acid diethylamide, or a pharmaceutically acceptable salt thereof;

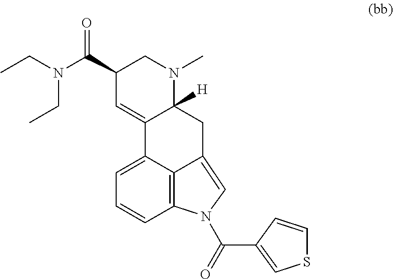 [SMILES: CCN(CC)C(=O)[C@@H]1C=C2c3cccc4c3c(cn4C(=O)c3ccsc3)C[C@H]2N(C)C1]

SYN-L-038, 1-(3-thiophenecarbonyl)-lysergic acid diethylamide, or a pharmaceutically acceptable salt thereof;

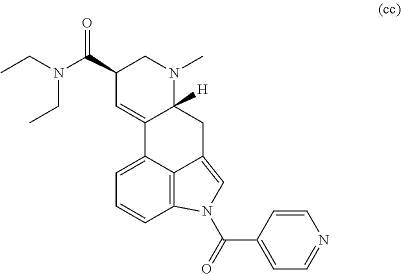 [SMILES: CCN(CC)C(=O)[C@@H]1C=C2c3cccc4c3c(cn4C(=O)c3ccncc3)C[C@H]2N(C)C1]

SYN-L-039, 1-isonicotinoyl-lysergic acid diethylamide, or a pharmaceutically acceptable salt thereof.

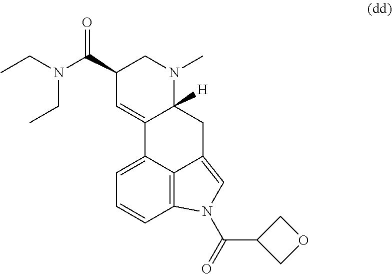 [SMILES: CCN(CC)C(=O)[C@@H]1C=C2c3cccc4c3c(cn4C(=O)C3COC3)C[C@H]2N(C)C1]

SYN-L-040, 1-(3-oxetanoyl)-lysergic acid diethylamide, or a pharmaceutically acceptable salt thereof;

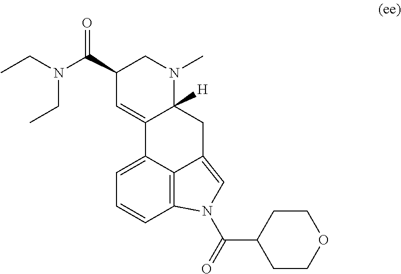 [SMILES: CCN(CC)C(=O)[C@@H]1C=C2c3cccc4c3c(cn4C(=O)C3CCOCC3)C[C@H]2N(C)C1]

SYN-L-041, 1-(4-tetrahydropyranoyl)-lysergic acid diethylamide, or a pharmaceutically acceptable salt thereof;

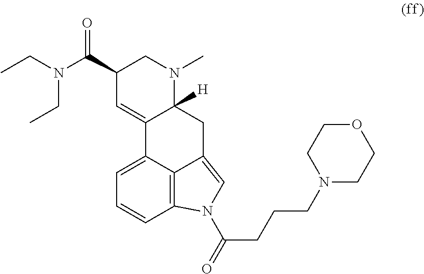 [SMILES: CCN(CC)C(=O)[C@@H]1C=C2c3cccc4c3c(cn4C(=O)CCCN3CCOCC3)C[C@H]2N(C)C1]

SYN-L-046, 1-(4-morpholinylbutyryl)-lysergic acid diethylamide, or a pharmaceutically acceptable salt thereof;

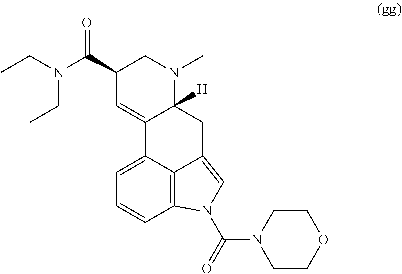 [SMILES: CCN(CC)C(=O)[C@@H]1C=C2c3cccc4c3c(cn4C(=O)N3CCOCC3)C[C@H]2N(C)C1]

SYN-L-047, 1-(4-morpholinecarbonyl)-lysergic acid diethylamide, or a pharmaceutically acceptable salt thereof;

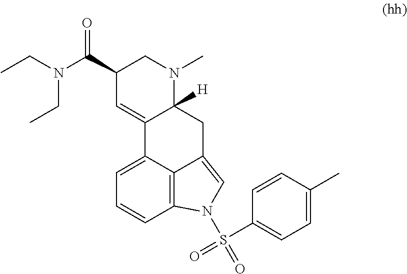 [SMILES: CCN(CC)C(=O)[C@@H]1C=C2c3cccc4c3c(cn4S(=O)(=O)c3ccc(C)cc3)C[C@H]2N(C)C1]

SYN-L-048 1-(p-tosyl)-lysergic acid diethylamide, or a pharmaceutically acceptable salt thereof;

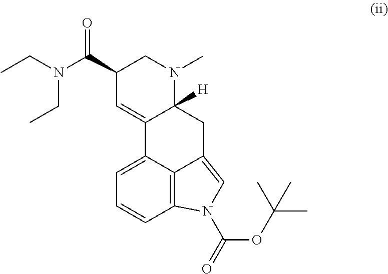 [SMILES: CCN(CC)C(=O)[C@@H]1C=C2c3cccc4c3c(cn4C(=O)OC(C)(C)C)C[C@H]2N(C)C1]

SYN-L-049, 1-(tert-butoxycarbonyl)-lysergic acid diethylamide, or a pharmaceutically acceptable salt thereof;

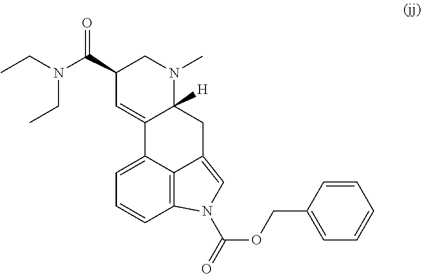 [SMILES: CCN(CC)C(=O)[C@@H]1C=C2c3cccc4c3c(cn4C(=O)OCc3ccccc3)C[C@H]2N(C)C1]

SYN-L-050, 1-(benzyloxycarbonyl)-lysergic acid diethylamide, or a pharmaceutically acceptable salt thereof.

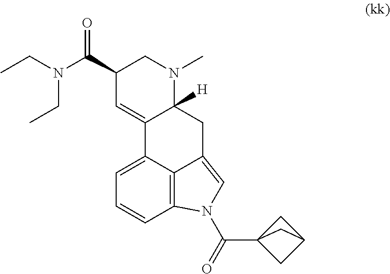 [SMILES: CCN(CC)C(=O)[C@@H]1C=C2c3cccc4c3c(cn4C(=O)C34CC(C3)C4)C[C@H]2N(C)C1]

SYN-L-051, 1-(bicyclo[1.1.1]pentylcarbonyl)-lysergic acid diethylamide, or a pharmaceutically acceptable salt thereof;

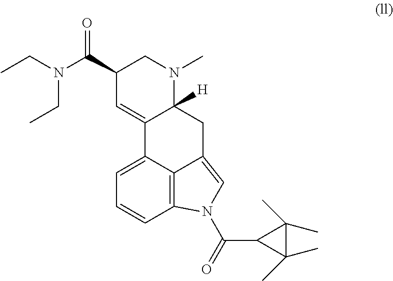 [SMILES: CCN(CC)C(=O)[C@@H]1C=C2c3cccc4c3c(cn4C(=O)C3C(C)(C)C3(C)C)C[C@H]2N(C)C1]

SYN-L-052, 1-(tetramethylcyclopropylmethanoyl)-lysergic acid diethylamide, or a pharmaceutically acceptable salt thereof;

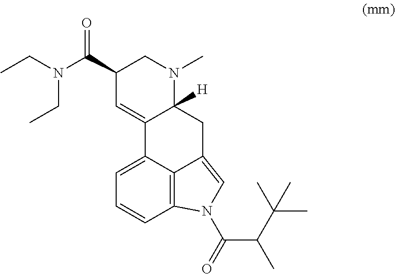 [SMILES: CCN(CC)C(=O)[C@@H]1C=C2c3cccc4c3c(cn4C(=O)C(C)C(C)(C)C)C[C@H]2N(C)C1]

SYN-L-053, 1-(2,3,3-trimethyl)butanoyl-lysergic acid diethylamide, or a pharmaceutically acceptable salt thereof;

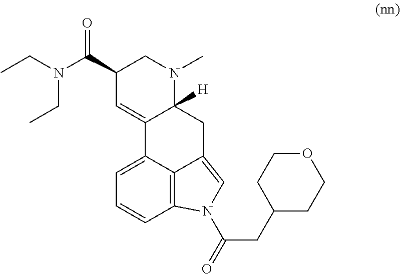 [SMILES: CCN(CC)C(=O)[C@@H]1C=C2c3cccc4c3c(cn4C(=O)CC3CCOCC3)C[C@H]2N(C)C1]

SYN-L-056, 1-(4-tetrahydropyranacetyl)-lysergic acid diethylamide, or a pharmaceutically acceptable salt thereof;

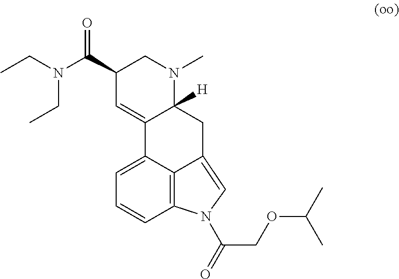 [SMILES: CCN(CC)C(=O)[C@@H]1C=C2c3cccc4c3c(cn4C(=O)COC(C)C)C[C@H]2N(C)C1]

SYN-L-060, 1-isopropoxyacetate-lysergic acid diethylamide, or a pharmaceutically acceptable salt thereof;

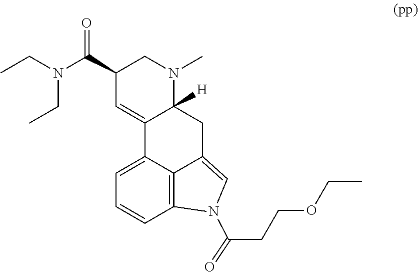 [SMILES: CCOCCC(=O)n1cc2c3c(cccc31)C1=C[C@@H](C(=O)N(CC)CC)CN(C)[C@@H]1C2]

SYN-L-061, 1-(3-ethoxypropanoyl)-lysergic acid diethylamide, or a pharmaceutically acceptable salt thereof;

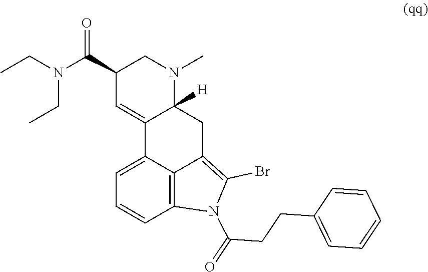 [SMILES: CCN(CC)C(=O)[C@@H]1C=C2c3cccc4c3c(c(Br)n4C(=O)CCc3ccccc3)C[C@H]2N(C)C1]

SYN-L-079, 1-(3-phenylpropionyl)-2-bromo-lysergic acid diethylamide, or a pharmaceutically acceptable salt thereof;

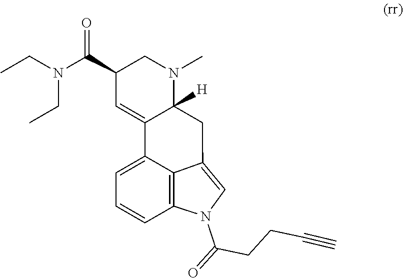 [SMILES: C#CCCC(=O)n1cc2c3c(cccc31)C1=C[C@@H](C(=O)N(CC)CC)CN(C)[C@@H]1C2]

SYN-L-191, 1-(pent-5-yn-1-oyl)-lysergic acid diethylamide, or a pharmaceutically acceptable salt thereof;

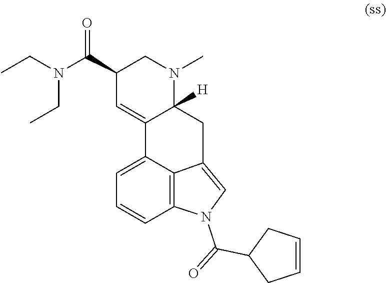 [SMILES: CCN(CC)C(=O)[C@@H]1C=C2c3cccc4c3c(cn4C(=O)C3CC=CC3)C[C@H]2N(C)C1]

SYN-L-192, 1-(cyclopent-3-enecarbonyl)-lysergic acid diethylamide, or a pharmaceutically acceptable salt thereof;

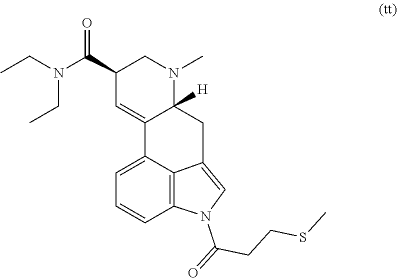 [SMILES: CCN(CC)C(=O)[C@@H]1C=C2c3cccc4c3c(cn4C(=O)CCSC)C[C@H]2N(C)C1]

SYN-L-193, 1-(3-thiomethylpropionyl)-lysergic acid diethylamide, or a pharmaceutically acceptable salt thereof;

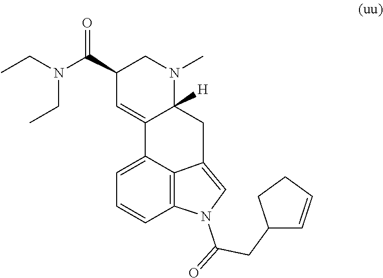 [SMILES: CCN(CC)C(=O)[C@@H]1C=C2c3cccc4c3c(cn4C(=O)CC3C=CCC3)C[C@H]2N(C)C1]

SYN-L-194, 1-(cyclopent-2-eneacetyl)-lysergic acid diethylamide, or a pharmaceutically acceptable salt thereof;

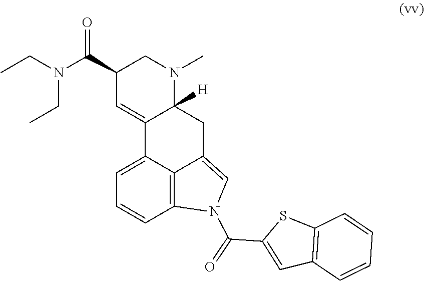 [SMILES: CCN(CC)C(=O)[C@@H]1C=C2c3cccc4c3c(cn4C(=O)c3cc4ccccc4s3)C[C@H]2N(C)C1]

SYN-L-217, 1-(2-benzothiophenecarbonyl)-lysergic acid diethylamide, or a pharmaceutically acceptable salt thereof;

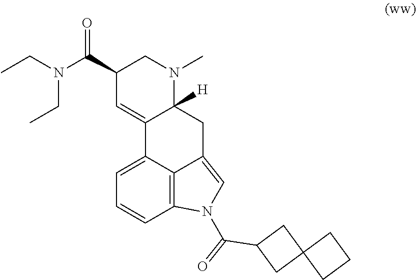 [SMILES: CCN(CC)C(=O)[C@@H]1C=C2c3cccc4c3c(cn4C(=O)C3CC4(CCC4)C3)C[C@H]2N(C)C1]

SYN-L-218, 1-spirobicyclobutanoyl-lysergic acid diethylamide, or a pharmaceutically acceptable salt thereof;

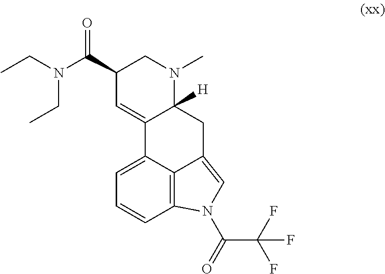 [SMILES: CCN(CC)C(=O)[C@@H]1C=C2c3cccc4c3c(cn4C(=O)C(F)(F)F)C[C@H]2N(C)C1]

SYN-L-219, 1-trifluoroacetyl-lysergic acid diethylamide, or a pharmaceutically acceptable salt thereof;

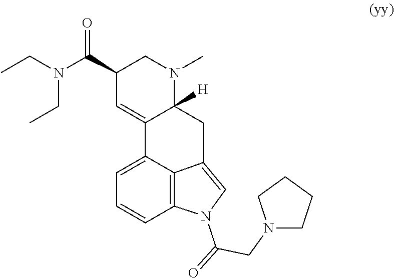 [SMILES: CCN(CC)C(=O)[C@@H]1C=C2c3cccc4c3c(cn4C(=O)CN3CCCC3)C[C@H]2N(C)C1]

SYN-L-220, 1-(N-pyrrolidine)acetyl-lysergic acid diethylamide, or a pharmaceutically acceptable salt thereof;

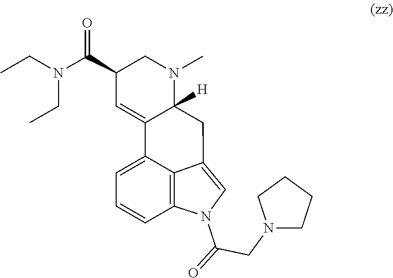 [SMILES: CCN(CC)C(=O)[C@@H]1C=C2c3cccc4c3c(cn4C(=O)CN3CCCC3)C[C@H]2N(C)C1]

SYN-L-221, 1-thienothiophenecarbonyl-lysergic acid diethylamide, or a pharmaceutically acceptable salt thereof;

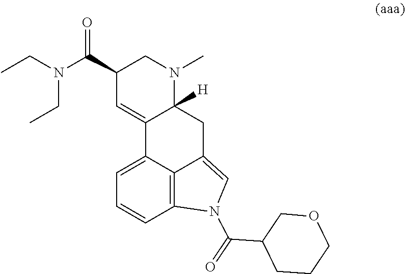 [SMILES: CCN(CC)C(=O)[C@@H]1C=C2c3cccc4c3c(cn4C(=O)C3CCCOC3)C[C@H]2N(C)C1]

SYN-L-223, 1-(3-tetrahydropyrancarbonyl)-lysergic acid diethylamide, or a pharmaceutically acceptable salt thereof;

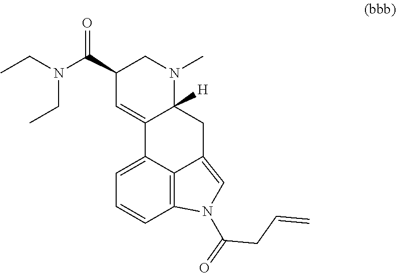 [SMILES: C=CCC(=O)n1cc2c3c(cccc31)C1=C[C@@H](C(=O)N(CC)CC)CN(C)[C@@H]1C2]

SYN-L-224, 1-(3-butenoyl)-lysergic acid diethylamide, or a pharmaceutically acceptable salt thereof;

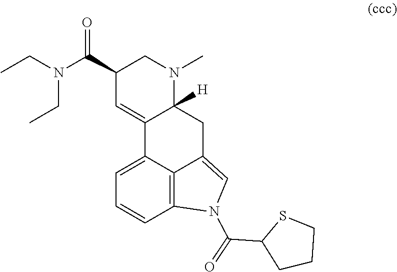 [SMILES: CCN(CC)C(=O)[C@@H]1C=C2c3cccc4c3c(cn4C(=O)C3CCCS3)C[C@H]2N(C)C1]

SYN-L-225, 1-(tetrahydro-2-thiophenecarbonyl)-lysergic acid diethylamide, or a pharmaceutically acceptable salt thereof;

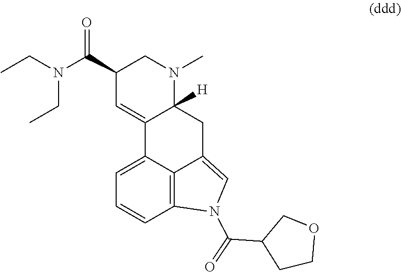 [SMILES: CCN(CC)C(=O)[C@@H]1C=C2c3cccc4c3c(cn4C(=O)C3CCOC3)C[C@H]2N(C)C1]

SYN-L-226, 1-(tetrahydro-3-furoyl)-lysergic acid diethylamide, or a pharmaceutically acceptable salt thereof;

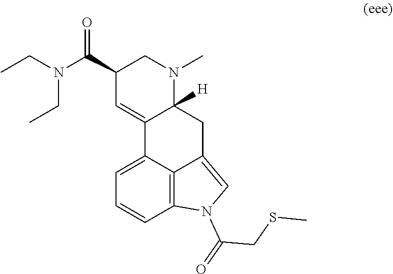 [SMILES: CCN(CC)C(=O)[C@@H]1C=C2c3cccc4c3c(cn4C(=O)CSC)C[C@H]2N(C)C1]

SYN-L-227, -(methylthio)acetyl-lysergic acid diethylamide, or a pharmaceutically acceptable salt thereof;

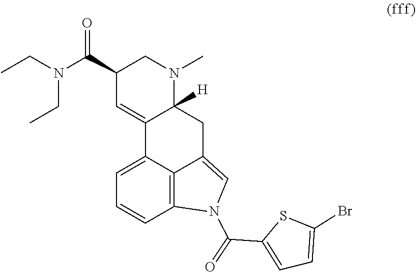 [SMILES: CCN(CC)C(=O)[C@@H]1C=C2c3cccc4c3c(cn4C(=O)c3ccc(Br)s3)C[C@H]2N(C)C1]

SYN-L-228, 1-(5-bromo-2-thiophenecarbonyl)-lysergic acid diethylamide, or a pharmaceutically acceptable salt thereof;

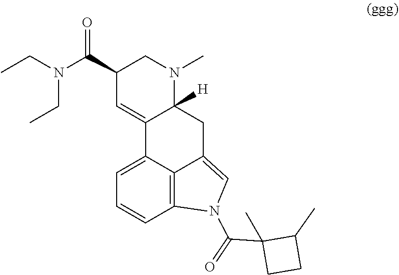 [SMILES: CCN(CC)C(=O)[C@@H]1C=C2c3cccc4c3c(cn4C(=O)C3(C)CCC3C)C[C@H]2N(C)C1]

SYN-L-229, 1-(1,2-dimethylcyclobutane-1-carbonyl)-lysergic acid diethylamide, or a pharmaceutically acceptable salt thereof;

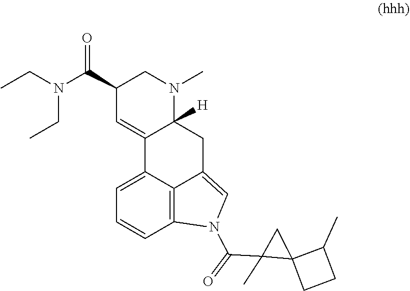 [SMILES: CCN(CC)C(=O)[C@@H]1C=C2c3cccc4c3c(cn4C(=O)C3(C)CC34CCC4C)C[C@H]2N(C)C1]

SYN-L-230, 1-(1,4-dimethylspiro[2.3]hexane-1-carbonyl)-lysergic acid diethylamide, or a pharmaceutically acceptable salt thereof;

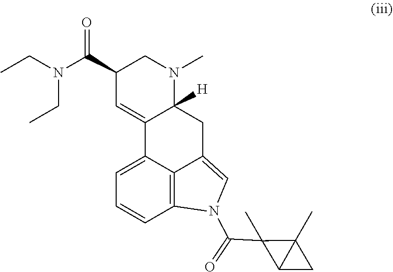 [SMILES: CCN(CC)C(=O)[C@@H]1C=C2c3cccc4c3c(cn4C(=O)C3(C)C4CC43C)C[C@H]2N(C)C1]

SYN-L-231, 1-(1,2-dimethylbicyclo[1.1.0]butane-2-carbonyl)-lysergic acid diethylamide or a pharmaceutically acceptable salt thereof;

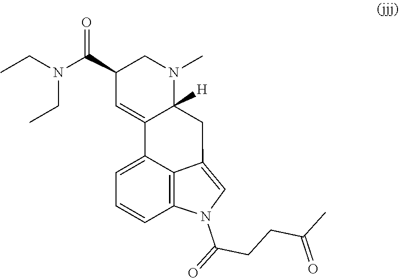 [SMILES: CCN(CC)C(=O)[C@@H]1C=C2c3cccc4c3c(cn4C(=O)CCC(C)=O)C[C@H]2N(C)C1]

SYN-L-232, 1-(4-oxopentanoyl)-lysergic acid diethylamide, or a pharmaceutically acceptable salt thereof;

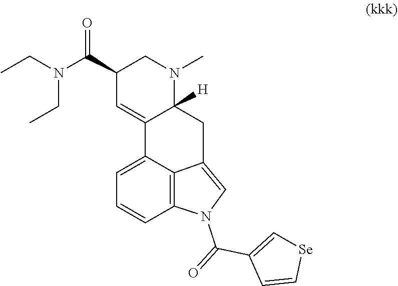 [SMILES: CCN(CC)C(=O)[C@@H]1C=C2c3cccc4c3c(cn4C(=O)c3cc[se]c3)C[C@H]2N(C)C1]

SYN-L-233, 1-(3-selenophenecarbonyl)-lysergic acid diethylamide, or a pharmaceutically acceptable salt thereof;

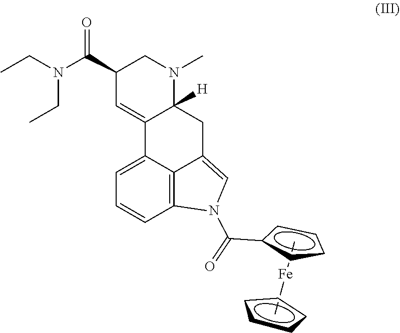

SYN-L-234, 1-(ferrocenecarbonyl)-lysergic acid diethylamide, or a pharmaceutically acceptable salt thereof; and

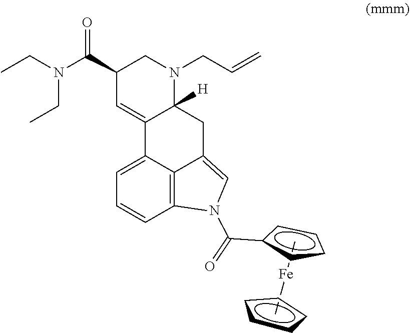

SYN-L-235, 1-(ferrocenecarbonyl)-6-allyl-6-nor-lysergic acid diethylamide, or a pharmaceutically acceptable salt thereof.

In a particularly preferred embodiment, the compound of the invention is selected from: SYN-L-005, SYN-L-010, SYN-L-012, SYN-L-013, SYN-017, SYN-L-028, SYN-036 and SYN-L-041, or a pharmaceutically acceptable salt thereof.

In a further preferred embodiment, the compound of the invention is selected from the compounds listed in Tables 1 and 2 as mentioned in the Examples, or a pharmaceutically acceptable salt or prodrug thereof.

Asymmetric centres may exist in the compounds of the invention. The asymmetric centres may be designated by the symbols “R” or “S”, depending on the configuration of substituents in three dimensional space at the chiral carbon atom.

All stereochemical isomeric forms, including diastereomeric, enantiomeric, and epimeric forms, as well as d-isomers and I-isomers, and mixtures thereof of the compounds are contemplated herein. Individual enantiomers of the compounds can be prepared synthetically from commercially available enantiopure starting materials or by preparing enantiomeric mixtures of the compounds and resolving the mixture into individual enantiomers. Resolution methods include conversion of the enantiomeric mixture into a mixture of diastereomers and separation of the diastereomers by, for example, recrystallisation or chromatography; direct separation of the enantiomers on chiral chromatographic columns; and any other appropriate method known in the art. Starting materials of defined stereochemistry may be commercially available or made and resolved by techniques well known in the art.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral centre(s). The prefixes d and I or (+) and (−) are employed to designate the sign of rotation of plane-polarized light by the compound, with (−) or I meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The compounds of the invention may also exist as geometric isomers. All cis, trans, syn, anti, (I) and (Z) isomers, as well as the appropriate mixtures thereof of the compounds are contemplated herein.

The compounds may also exist as tautomers, for example, keto/enol; imine/enamine; amide/imino alcohol; nitroso/oxime; thioketone/enethiol; N-nitroso/hydroxyazo; and nitro/aci-nitro. All tautomeric isomers of the compounds are contemplated herein.

The compounds may also exist as isotopologues and isotopomers, wherein one or more atoms in the compounds are replaced with different isotopes. Suitable isotopes include, for example, ¹H, ²H (D), ³H (T), ¹²C, ¹³C, ¹⁴C, ¹⁶O, ¹⁸O, ¹⁸F and ¹⁹F.

The compounds may exist in solvated or unsolvated forms. If the solvent is water, the solvate may be referred to as a hydrate, for example, a mono-hydrate, a di-hydrate, or a tri-hydrate. All solvates of the compounds are contemplated herein. Salts or pharmaceutically acceptable salts of the compounds of the invention are also contemplated herein. Salts of the compounds include, for example, acid addition salts, base addition salts, and quaternary salts of basic nitrogen-containing groups.

Acid addition salts can be prepared by reacting compounds, in free base form, with inorganic or organic acids. Examples of inorganic acids include, but are not limited to, hydrochloric; hydrobromic; hydroiodic; nitric; carbonic; sulfuric; and phosphoric acid. Examples of organic acids include, but are not limited to, cholic; sorbic; lauric; acetic; trifluoroacetic; formic; propionic; succinic; glycolic; gluconic; digluconic; lactic; malic; tartaric; citric; ascorbic; glucuronic; maleic; fumaric; pyruvic; aspartic; glutamic; aryl carboxylic; anthranilic acid; mesylic; stearic; salicylic; phenylacetic; mandelic; embonic (pamoic); alkylsulfonic; ethanesulfonic; arylsulfonic; benzenesulfonic; pantothenic; sulfanilic; cyclohexylaminosulfonic; P-hydroxybutyric; galactaric; galacturonic; adipic, alginic; butyric; camphoric; camphorsulfonic; cyclopentanepropionic; dodecylsulfic; glycoheptanoic; glycerophosphic; heptanoic; hexanoic; nicotinic; 2-naphthalesulfonic; oxalic; palmoic; pectinic; 3-phenylpropionic; picric; pivalic; thiocyanic; tosylic; and undecanoic acid.

Base addition salt can be prepared by reacting compounds, in free acid form, with inorganic or organic bases. Examples of base addition salts include metal salts and organic salts. Preferred metal salts include alkali metal salts, alkaline earth metal salts, and other physiologically acceptable metal salts. Preferably the metal salt comprises aluminium, calcium, lithium, magnesium, potassium, sodium, or zinc. Organic salts may be made from amines, such as trimethylamine, diethylamine, N,N′-dibenzylethylenediamine, N′,N′-dibenzylethylenediamine, chloroprocaine, ethanolamine, diethanolamine, ethylenediamine, meglumine (N-methylglucamine), and procaine.

### III. Compounds for Use in Treating Diseases and Methods for Treating Diseases Utilizing the Compounds

The third and fourth aspect of the invention pertain to a compound of formula (1) or a pharmaceutically acceptable salt thereof, for use in treating diseases, requiring modulated or reduced hallucinogenic side effects as compared to LSD and 1-acetyl-LSD, in a subject, and to a method for treating diseases requiring a modulated or reduced hallucinogenic side effects as compared to LSD and 1-acetyl-LSD, said method comprising administering a compound of formula (1), or a pharmaceutically acceptable salt thereof to a subject or patient in need of such treatment. The compound/method is particularly suitable for treating anxiety in a subject, for treating depression in a subject, for treating migraine headache in a subject, for treating cluster headache in a subject, for treating glaucoma in a subject, for treating macular degeneration in a subject, for treating rheumatoid arthritis in a subject and for treating Alzheimer's disease in a subject.

Apart for the compounds mentioned as preferred embodiments of the first aspect above, the following compounds of formula (1) are preferably utilized in the third and fourth aspect of the invention:
Compounds of formula (1), wherein R² is H, R⁶ is methyl, R^{a}═R^{b}=ethyl, [symbol] is a double bond, X is C═O, Y is a bond or a C₁₋₄ alkylene residue, which may be substituted with C₁₋₃ alkyl or halogen, and Z is selected from hydrogen, C₁₋₁₂ alkyl, C₂₋₁₂ alkenyl, C₂₋₁₂ alkynyl, C₃₋₇ cycloalkyl, C₄₋₇ cycloalkenyl, C₁₋₁₂ alkoxy, aryl, heterocyclyl, heteroaryl, COOH, and CO—C₁₋₆ alkyl, and
Compounds of formula (1) selected from 1-acetyl-lysergic acid morpholide (SYN-L-196), 1-propionyl-6-methallyl-6-nor-lysergic acid diethylamide (SYN-L-203), 1-propionyl-6-allyl-6-nor-lysergic acid methylisopropylamide (SYN-L-073), 1-propionyl-2-bromo-lysergic acid diethylamide (SYN-L-017) and 1-cyclopropanesulfonyl-lysergic acid diethylamide (SYN-L-014).

Furthermore, compounds of formula (1) having a C₂₋₅ acyl group as —XYZ, which are excluded from the compounds of the first aspect by means of a disclaimer, may be used in the third and fourth aspect of the invention. Such compounds preferably include 1-propionoyl-lysergic acid diethylamide (1P-LSD), 1-butanoyl-lysergic acid diethylamide (1B-LSD), 1-cyclopropylcarbonyl-lysergic acid diethylamide (1 cP-LSD), 1-valeroyl-lysergic acid diethylamide (1V-LSD), 1-propionoyl-6-allyl-6-nor-lysergic acid diethylamide (1P-AL-LAD), 1-cyclopropanecarbonyl-6-allyl-6-nor-lysergic acid diethylamide (1 cP-AL-LAD), 1-propionoyl-6-ethyl-6-nor-lysergic acid diethylamide (1P-ETH-LAD), 1-propionyl-lysergic acid methylisopropylamide (1P-MIPLA) and 1-cyclopropanecarbonyl-lysergic acid methylisopropylamide (1 cP-MIPLA).

### IV. Methods of Making the Compounds of the Invention

The abbreviations used in the general methods are defined as follows: “LG” is an appropriate leaving group and includes halo, trifluoromethyl, tosyl, brosyl, nosyl, mesyl, alkylsulfonyloxy, trifluoroalkylsulfonyloxy, arylsulfonyloxy, fluorosulfonyl, triflate, chlorosulfite, phosphite ester, imidazolyl, triazolyl, tetrazolyl, and any other highly stabilised leaving group. Such leaving groups are known to those of skill in the art.

“AcOH” means acetic acid. “BrCN” means cyanogen bromide. “CCl₄” means carbon tetrachloride. “CHCl₃” means chloroform. “CH₃CN” means acetonitrile. “Cu(OAc)₂” means copper (II) acetate. “DABCO” means 1,4-diazabicyclo[2.2.2]octane. “DBN” means 1,5-diazabicyclo[4.3.0]non-5-ene. “DBU” means 1,8-diazabicyclo-[5.4.0]undec-7-ene. “DCM” means dichloromethane (CH2Cl2). “DMAP” means 4-(dimethylamino)pyridine. “DMF” means dimethylformamide. “Et” means ethyl. “EtOAc” means ethyl acetate. “H₂O” means water. “K₂CO₃” means potassium carbonate. “K₃PO₄” means potassium phosphate. “Me” means methyl (—CH₃). “MeOH” means methanol. “NaOH” means sodium hydroxide. “Na₂S₂O₄” means sodium dithionite. “NH₃” means ammonia. “Pd(dppf)Cl₂” means [1,1′-bis(diphenylphosphino)ferrocene]dichloropalladium(II). “POCl₃” means phosphoryl trichloride. “Pr” means propyl. “i-Pr” means isopropyl (—CH(CH₃)₂). “iPrOH” means isopropanol. “rt” means room temperature. “RuCl3” means ruthenium (III) chloride. “TBAHSO₄” means tetrabutylammonium hydrogen sulfate. “tBuOK” means potassium tert-butoxide. “TEAOH” means tetraethylammonium hydroxide. “THF” means tetrahydrofuran. “TLC” means thin layer chromatography. “Zn” means zinc metal. “Zn(CH₃)₂” means dimethylzinc.

In the context of the present invention, protecting groups include prodrug moieties and chemical protecting groups.

Protecting groups (PG) are optionally used to prevent side reactions with the protected group during synthetic procedures, i.e. routes or methods to prepare the compounds of the invention. For the most part the decision as to which groups to protect, when to do so, and the nature of the chemical PG will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the person skilled in the art.

Various functional groups of the compounds of the invention may be protected. For example, PGs for —OH groups (whether hydroxyl, carboxylic acid, phosphonic acid, or other functions) include “ether- or ester-forming groups”. Ether- or ester-forming groups are capable of functioning as chemical PGs in the synthetic schemes set forth herein. However, some hydroxyl and thio PGs are neither ether-nor ester-forming groups, as will be understood by those skilled in the art, and are included with amides, discussed below.

A very large number of hydroxyl PGs and amide-forming groups and corresponding chemical cleavage reactions are described in Protective Groups in Organic Synthesis, Theodora W. Greene and Peter G. M. Wuts (John Wiley & Sons, Inc., New York, 1999, ISBN 0-471-16019-9) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For PGs for carboxylic acid, phosphonic acid, phosphonate, sulfonic acid and other PGs for acids see Greene as set forth below. Such groups include by way of example and not limitation, esters, amides, hydrazides, and the like.

Ester-forming groups include: (1) phosphonate ester-forming groups, such as phosphonamiditeate esters, phosphorothioate esters, phosphonate esters, and phosphon-bis-amidates; (2) carboxyl ester-forming groups, and (3) sulphur ester-forming groups, such as sulphonate, sulfate, and sulfinate.

The compounds of the invention can be synthesised according to the General Methods A to F described below.

### IV.A. General Method A

Compounds of formula (1) can be prepared using the synthetic route detailed in Scheme 1, in which, R^{a}, R^{b}, R², R⁶, X, Y, Z and [symbol] are as defined for the invention and LG is an appropriate leaving group, typically chloro or bromo. Base is selected from an alkali hydroxide, typically sodium hydroxide or potassium hydroxide. PTC (phase-transfer catalyst) is selected from an appropriate tetraalkylammonium salt, typically tetrabutylammonium hydrogensulfate. It will be apparent to those skilled in the art that other similar phase-transfer catalysts could also be employed.

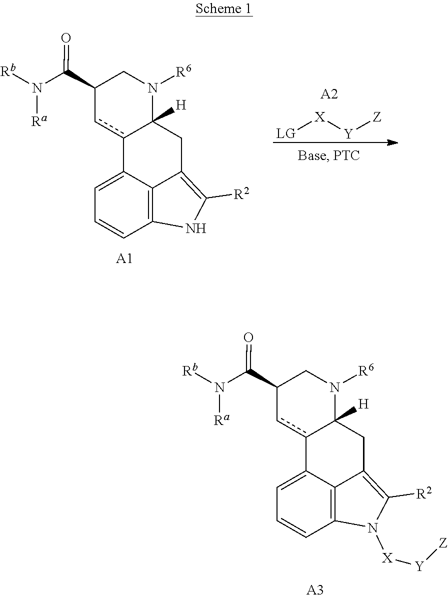

Compounds of formula A1 can be obtained from specialist commercial suppliers or synthesised as needed by methods described herein or as described in the wider literature and known to those skilled in the art. Compounds of formula A2 can be obtained from standard commercial suppliers or synthesised as needed by methods already described in the literature and known to those skilled in the art.

Compounds of formula A1 are dissolved in a halogenated solvent, typically dichloromethane or dichloroethane at room temperature and under a nitrogen atmosphere. With vigorous stirring, PTC and powdered base is added forming a biphasic mixture. The reaction is cooled to −10° C. and the appropriate compound of formula A2 is added in a dropwise manner such that the reaction temperature does not exceed −5° C. After stirring at −10° C. to 15° C. for 1 to 8 h, the reaction mixture is filtered over celite and extracted with a halogenated solvent. The organic phase is washed with water and brine and then dried over magnesium sulphate before concentrating under vacuum. The resulting residue, a compound of formula A3, is then purified by processes known to the art such as flash column chromatography, crystallisation or preparative HPLC. The General Method A is further explained in Examples 1 and 2 below.

### IV.B. General Method B

Compounds of formula (1) can be prepared using the synthetic route detailed in Scheme 2, in which, R^{a}, R^{b}, R², R⁶, X, Y, Z and [symbol] are as defined for the invention (Z may be a leaving group such as halogen that allows for a replacement with a different substituent), and LG is an appropriate leaving group, typically chloro or bromo. Base is selected from a strong base, typically n-butyllithium. It will be apparent to those skilled in the art that other similar bases could also be employed.

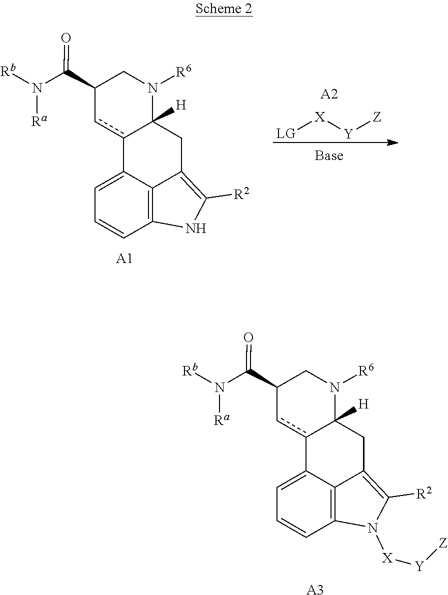

Compounds of formula A1 can be obtained from specialist commercial suppliers or synthesised as needed by methods described herein or as described in the wider literature and known to those skilled in the art. Compounds of formula A2 can be obtained from standard commercial suppliers or synthesised as needed by methods already described in the literature and known to those skilled in the art.

Compounds of formula A1 are dissolved in anhydrous tetrahydrofuran and cooled to −78° C. under a nitrogen atmosphere. With stirring, base is added in a dropwise manner such that the reaction temperature does not exceed −70° C. After stirring for 30-120 min, the appropriate compound of formula A2 is then added in a dropwise manner such that the reaction temperature does not exceed −70° C. The resulting mixture is stirred at −78° C. overnight before careful addition of water. The aqueous phase is extracted with dichloromethane and the organic phase washed with water and brine and then dried over magnesium sulphate before concentrating under vacuum. The resulting residue, a compound of formula A3, is then purified by processes known to the art such as flash column chromatography, crystallisation or preparative HPLC. The General Method B is further explained in Examples 3 to 5 below.

### IV.C. General Method C

Compounds of formula (1) can be prepared using the synthetic route detailed in Scheme 3, in which, R^{a}, R^{b}, R², R⁶, X, Y, Z and [symbol] are as defined for the invention and LG is an appropriate leaving group, typically chloro, bromo, imidazole or triazoles. Base can be selected from; DMAP, triethylamine, DBU or DABCO. It will be apparent to those skilled in the art that other similar bases could also be employed.

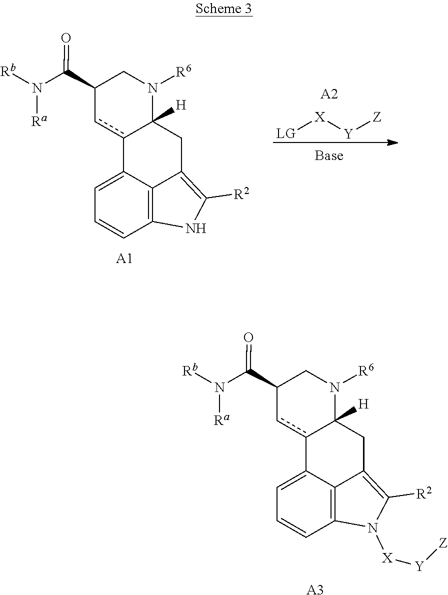

Compounds of formula A1 can be obtained from specialist commercial suppliers or synthesised as needed by methods described herein or as described in the wider literature and known to those skilled in the art. Compounds of formula A2 can be obtained from standard commercial suppliers or synthesised as needed by methods already described in the literature and known to those skilled in the art.

Compounds of formula A1 are dissolved in anhydrous acetonitrile at room temperature under a nitrogen atmosphere. With stirring, the appropriate compound of formula A2 and base is added forming a solution. The reaction is heated at 20° C. to reflux for 1 to 24 h and then concentrated under vacuum. The residue is dissolved in dichloromethane, washed with saturated sodium bicarbonate solution, water and brine and then dried over magnesium sulphate before concentrating under vacuum. The resulting residue, a compound of formula A3, is then purified by processes known to the art such as flash column chromatography, crystallisation or preparative HPLC. The General Method C is further explained in Examples 6 and 7 below.

### IV.D. General Method D

Compounds of formula (1) can be prepared using the synthetic route detailed in Scheme 4, in which, R^{a}, R^{b}, R², R⁶, X, Y, Z are as defined for the invention and [symbol] is a double bond. LG is an appropriate leaving group, typically chloro, bromo or iodo. Base is selected from a metal carbonate, typically potassium carbonate. The last step in the procedure, to obtain compounds of formula A10, can employ either that described in Scheme 1, that described in Scheme 2 or that described in Scheme 3.

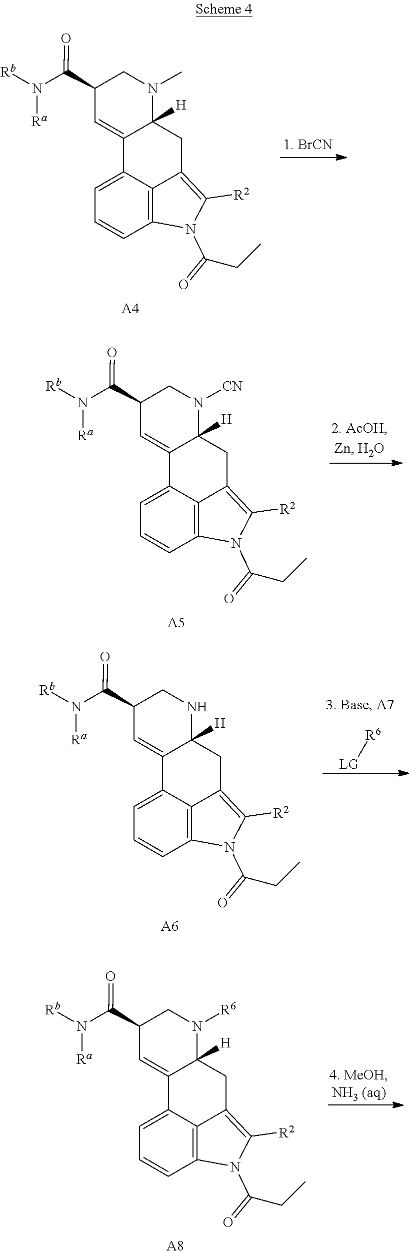

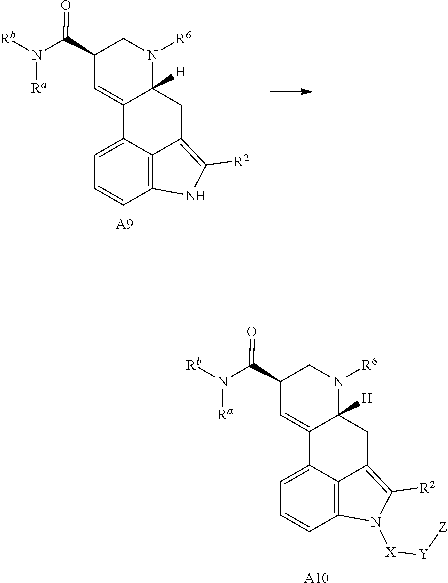

Compounds of formula A4 can be obtained from specialist commercial suppliers or synthesised as needed by methods described herein or as described in the wider literature and known to those skilled in the art. Compounds of formula A7 can be obtained from standard commercial suppliers or synthesised as needed by methods already described in the literature and known to those skilled in the art. In the first step, a compound of formula A4 is dissolved in chloroform at room temperature under a nitrogen atmosphere. With stirring, cyanogen bromide is carefully added forming a dark mixture. The reaction is heated at reflux for 2 to 12 h before cooling to room temperature and filtering over celite. The celite is extracted with further portions of chloroform and the combined organics are concentrated under vacuum to dryness. The resulting solid, a compound of formula A5, is either used directly in the next step or purified by processes known to the art such as flash column chromatography, crystallisation or preparative HPLC.

In the second step, a compound of formula A5 is dissolved in glacial acetic acid at room temperature under a nitrogen atmosphere. With stirring, water and finely powder zinc is carefully added forming a suspension. The reaction is heated at reflux for 2 to 12 h before cooling to room temperature. With continued stirring, the reaction mixture is diluted with water and chilled before a portion of dichloromethane is added. Concentrated aqueous ammonia is added in a dropwise manner until a pH of 9 to 10 is maintained. The phases are allowed to separate and the aqueous layer is extracted with further portions of dichloromethane. The combined organics are washed with water and brine and then dried over magnesium sulphate before concentrating under vacuum. The resulting residue, a compound of formula A6, is either used directly in the next step or purified by processes known to the art such as flash column chromatography, crystallisation or preparative HPLC.

In the third step, a compound of formula A6 is dissolved in anhydrous acetonitrile or anhydrous N,N-dimethylformamide at room temperature under a nitrogen atmosphere. Anhydrous potassium carbonate and the appropriate compound of formula A7 is added forming a suspension. This is heated at 20° C. to reflux for 1 to 12 h before concentrating under hard vacuum. The residue is taken up in dichloromethane, filtered over celite and the organics dried over magnesium sulphate before concentrating under vacuum. The resulting residue, a compound of formula A8, is purified by processes known to the art such as flash column chromatography, crystallisation or preparative HPLC.

In the fourth step, a compound of formula A8 is dissolved in methanol at room temperature under a nitrogen atmosphere. With stirring, concentrated aqueous ammonia is added and the homogenous mixture heated between 20° C. to 50° C. for 2 to 24 h. After cooling to room temperature, the methanol is removed under vacuum effectuating the precipitation of a gummy residue. The mixture is taken up in dichloromethane and the phases allowed to separate. The aqueous phase is extracted with further portions of dichloromethane and the combined organics are dried over magnesium sulphate before concentrating under vacuum. The resulting residue, a compound of formula A9, is either used directly in the next step or purified by processes known to the art such as flash column chromatography, crystallisation or preparative HPLC. The General Method D is further explained in Examples 8 and 9 below.

### IV.E. General Method E

Compounds of formula (1) can also be prepared using the synthetic route detailed in Scheme 5, in which R^{a}, R^{b}, X, Y, Z are as defined for the invention, R² is bromo, R⁶ is methyl and [symbol] is a double bond. The first two steps of the method depicted in Scheme 5 represent the fifth aspect of the invention as described hereinbefore, the compounds A12 and A14 corresponding with the compounds of formulas (3) and (4) of the method of the fifth aspect. Of note, an alternative production method of the compound of formula (3)/A12 by bromination of a specific lysergic acid amide is described in US 2016/0237080 A1. In a preferred embodiment of the method of the fifth aspect, the initial saponification step comprises reaction in an aqueous medium with a base in the presence of sodium dithionite and a (PTC) phase transfer catalyst. The base may be selected from an alkali hydroxide, typically sodium hydroxide or potassium hydroxide, and the PTC (phase-transfer catalyst) may be selected from an appropriate tetraalkylammonium salt, typically tetrabutylammonium hydroxide or tetraethylammonium hydroxide. It will be apparent to those skilled in the art that other similar selective saponification systems and phase-transfer catalysts could also be employed. In a further preferred embodiment of the method of the fifth aspect, the second amide-formation step comprises reaction of the compound of formula (3) with HNR^{a}R^{b} in the presence of a carboxylic acid activating agent, such as POCl₃.

The last step in the procedure, to obtain compounds of formula A15, can employ either that described in Scheme 1, that described in Scheme 2 or that described in Scheme 3.

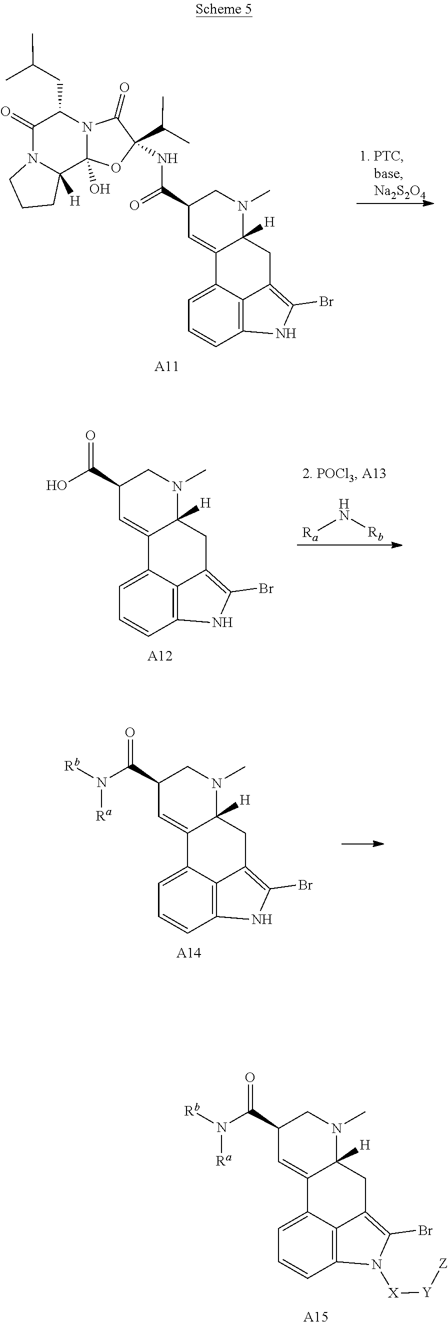

The compound of formula A13 can be obtained from standard commercial suppliers or synthesised as needed by methods already described in the literature and known to those skilled in the art. The compound of formula A11 is known as bromocriptine, an important ergoline dopamine agonist used in the treatment of Parkinson's disease and several other diseases. As such, bromocriptine is a widely distributed medication and available cheaply and easily from commercial suppliers without the need for any controlled substance licenses. An important feature of this invention, as described by General Method E and Scheme 5, is the use of bromocriptine or it's salts to access several compounds of formula (1) as well as intermediates and compounds including, but not limited to those described by formulas A12, A14 and A15. The use of bromocriptine to access these molecules provides a cost-effective, robust and flexible method of synthesis with a good yield. In the first step, PTC, base and sodium dithionite are dissolved in water at room temperature under a nitrogen atmosphere. With stirring, compound of formula A11 is added in small portions forming a mixture. The reaction is heated to reflux for 2 to 12 h before cooling to room temperature. The resulting dark solution is acidified with acetic acid until a pH of 6 to 6.5 is maintained. With continued stirring, the reaction is cooled to 0° C. to 5° C. for 2 to 12 h, effectuating precipitation. The mixture is filtered under vacuum and the filter cake washed with several portions of water and then isopropanol. The filter cake is dried in the oven at 50° C. to 100° C. for 1 to 4 days under hard vacuum. The resulting solid, compound of formula A12, is either used directly in the next step or purified by processes known to the art such as flash column chromatography, crystallisation or preparative HPLC.

In the second step, compound of formula A12 is suspended in chloroform at room temperature under a nitrogen atmosphere. With stirring, a compound of formula A13 is added in one portion. The reaction is heated to 20° C. to 50° C. and phosphorus oxychloride is added in a dropwise manner such that reflux is maintained. After the addition, the reaction is stirred for an additional 20 min to 120 min. With continued stirring, water and then concentrated aqueous ammonia is added such that the mixture is sufficiently basic. The layers are allowed to separate and the aqueous phase is extracted with dichloromethane. The combined organics are washed with water and brine and then dried over magnesium sulphate before concentrating under vacuum. The resulting residue, compound of formular A14, is purified by processes known to the art such as flash column chromatography, crystallisation or preparative HPLC. The general method E is further explained in Examples 10 and 11 below

To access further R² substituted derivatives, as described in formula (1), where R² is as defined for the invention, further transformations can be made using compounds of formula A14 via methods described in the literature and known to those skilled in the art. These compounds can then be further functionalised as per Schemes 1, 2, 3, 4, 6 and 7, as appropriate.

### IV. F. General Methods—Transformations for Lysergamides

Compounds of formula (1) can be prepared using the synthetic route detailed in Scheme 6, in which, R^{a}, R^{b}, R², R⁶, X, Y and Z are as defined for the invention and [symbol] is a single bond. The last step in the procedure, to obtain compounds of formula A17, can employ either that described in Scheme 1, that described in Scheme 2 or that described in Scheme 3.

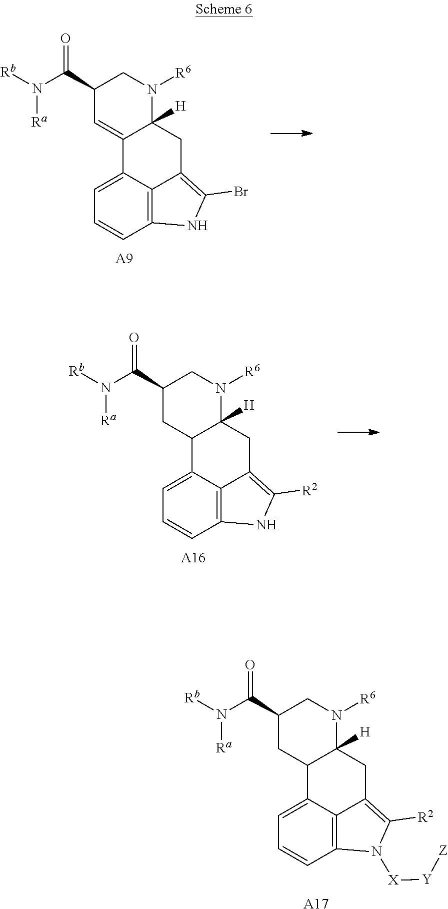

To access compounds of formula A17, further transformations can be made using compounds of formula A9 by employing the same hydrogenation strategy as described by Wagger et al. (Wagger J, et al. Synthesis of European pharmacopoeial impurities A, B, C, and D of cabergoline. RSC Adv., 2013, 3, 23146-23156. DOI: 10.1039/C₃RA43417F) or via other methods described in the literature and known to those skilled in the art. Such General Method F is further explained in Example 12 below. These compounds can then be further functionalised as per Schemes 1, 2 and 3, as appropriate.

Furthermore, compounds of formula (1) can be prepared using the synthetic route detailed in Scheme 7, in which, R^{a}, R^{b}, R², R⁶, X, Y and Z are as defined for the invention and [symbol] is a cyclopropyl group. The last step in the procedure, to obtain compounds of formula A19, can employ either that described in Scheme 1, that described in Scheme 2 or that described in Scheme 3.

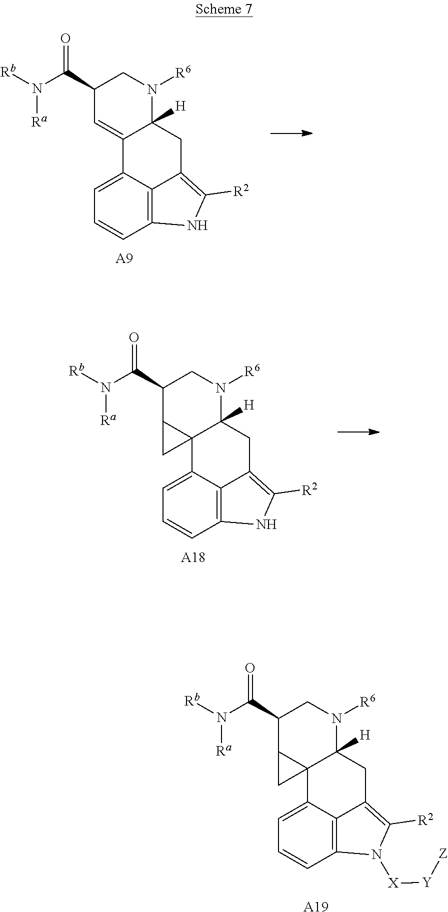

To access compounds of formula A19, further transformations can be made using compounds of formula A9 by employing the same cyclopropanation strategy as described by Incze et al. (Incze M, et al. Cyclopropanation of Carbon-Carbon Double Bonds in Ring D of Ergot Alkaloids. Heterocycles 2013; 87(7): 1553-1559. DOI: 10.3987/COM-13-12735) or via other methods described in the literature and known to those skilled in the art. Such General Method F is further explained in Example 13 below. These compounds can then be further functionalised as per Schemes 1, 2 and 3, as appropriate.

### V. Pharmaceutical Compositions

The compounds of formula (1) utilized in the pharmaceutical composition of the second aspect of the invention and the compounds/medicaments for use in treating diseases of the third aspect of the invention (hereinafter shortly referred to as “pharmaceutical composition of the invention” and “medicament of the invention”) and the medical treatment of the fourth aspect of the invention can be used in a range of medicinal applications as described hereinbefore. While it is possible for the active ingredients to be administered alone (which is also contemplated as a separate aspect of the invention), it may be preferable to present them as pharmaceutical compositions. The pharmaceutical compositions and medicaments of the invention comprise one or more compounds of the present invention, together with one or more acceptable carriers and optionally other therapeutic ingredients. The carriers must be “acceptable” in the sense of being compatible with the other ingredients of the composition and physiologically innocuous to the recipient. The carrier must also be suitable for the mode of administration of the active agent.

In the second aspect, the invention relates to a pharmaceutical composition comprising a compound of the invention, or a pharmaceutically acceptable salt and a pharmaceutically acceptable carrier. Pharmaceutical compositions containing the active ingredient may be in any form suitable for the intended method of administration. When used for oral use for example, gel, caps, tablets, troches, lozenges, aqueous or oil suspensions, dispersible powders or granules, emulsions, hard or soft capsules, syrups or elixirs may be prepared. Compositions intended for oral use may be prepared according to any method known in the art for the manufacture of pharmaceutical compositions. Such compositions may contain one or more agents including sweetening agents, flavouring agents, colouring agents and preserving agents, in order to provide a palatable preparation.

Compositions intended for buccal or sublingual use may include orally disintegrating tablets or wafers which may be prepared by methods known to those skilled in the art. Compositions intended for intranasal use may include nasal sprays or nebuliser formulations which may be prepared by methods known to those skilled in the art. Compounds intended for injection may include aqueous formulations for intravenous use and lipophilic formulations for intramuscular depot injection, which may be prepared by methods known to those skilled in the art.

The compounds of this invention are formulated with conventional carriers and excipients, which will be selected in accord with ordinary practice. Tablets will contain carriers, excipients, glidants, fillers, binders and the like. Aqueous compositions are prepared in sterile form, and when intended for delivery by other than oral administration generally will be isotonic. All compositions will optionally contain excipients such as those set forth in the Handbook of Pharmaceutical Excipients (1986), herein incorporated by reference in its entirety. Carriers and excipients include methylsulfonylmethane (MSM), 2-hydroxypropyl beta-cyclodextrin, mannitol, ascorbic acid and other antioxidants, chelating agents such as EDTA, carbohydrates such as dextrin, hydroxyalkylcellulose, hydroxyalkylmethylcellulose, stearic acid and the like. The pH of the compositions ranges from about 3 to about 11 but is ordinarily about 7 to 10.

The pharmaceutical composition and medicaments of the invention may exist in a suspension. Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more colouring agents, one or more flavouring agents and one or more sweetening agents, such as sucrose or saccharin.

Dispersible powders and granules suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavouring and colouring agents, may also be present.

Oil suspensions may be formulated by suspending the active ingredient in a vegetable oil, such as arachis oil, olive oil, sesame oil or coconut oil, or in a mineral oil such as liquid paraffin. The oil suspensions may contain a thickening agent, such as beeswax, hard paraffin or cetyl alcohol. Sweetening agents, such as those set forth herein, and flavouring agents may be added to provide a palatable oral preparation. These compositions may be preserved by the addition of an antioxidant such as ascorbic acid.

The pharmaceutical compositions and medicaments of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavouring agents. Syrups and elixirs may be formulated with sweetening agents, such as glycerol, sorbitol or sucrose. Such compositions may also contain a demulcent, a preservative, a flavouring or a colouring agent.

The oily phase of the emulsions may be constituted from known ingredients in a known manner. While the phase may comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and fat make up the so-called emulsifying ointment base which forms the oily dispersed phase of cream compositions.

Emulgents and emulsion stabilisers suitable for use in the composition and medicaments of the invention include Tween® 20 or 60, Span® 80, cetostearyl alcohol, benzyl alcohol, myristyl alcohol, glyceryl mono-stearate and sodium lauryl sulfate.

The invention is furthermore described in the following Examples, which shall, however, not be construed as limiting the present invention. The aspects and embodiments of the invention described above and described in the following examples are intended to be merely exemplary, and those skilled in the art will recognise, or will be able to ascertain using no more than routine experimentation, numerous equivalents of specific materials, compounds, and procedures. All such equivalents are considered to be within the scope of the invention as set out in the accompanying claims.

### EXAMPLES

### Example 1—SYN-L-027 (1-hexanoyl-lyserqic acid diethylamide)

d-lysergic acid diethylamide (500 mg, 1.55 mmol, 1 eq) was dissolved in dichloromethane (30 ml) at room temperature and under a nitrogen atmosphere. With vigorous stirring, tetrabutylammonium hydrogensulfate (316 mg, 0.930 mmol, 0.6 eq) and powdered dry sodium hydroxide (3100 mg, 77.5 mmol, 50 eq) was added forming a biphasic mixture. The reaction was cooled to −10° C. and hexanoyl chloride (626 mg, 4.65 mmol, 3 eq) was added in a dropwise manner such that the reaction temperature did not exceed −5° C. After stirring at −10° C. for 3 h, the reaction mixture was filtered over celite and extracted with a dichloromethane (2×20 ml). The organic phase was washed with water (2×15 ml) and brine (1×15 ml) and then dried over magnesium sulphate before concentrating under vacuum. The resulting residue, was then purified by silica gel flash column chromatography using a dichloromethane/methanol gradient. There was obtained 415 mg (65%) of SYN-L-027 as a brown oil.

### Example 2—SYN-L-008 (1-(o-toluoyl)-lyserqic acid diethylamide)

d-lysergic acid diethylamide (500 mg, 1.55 mmol, 1 eq) was dissolved in dichloromethane (30 ml) at room temperature and under a nitrogen atmosphere. With vigorous stirring, tetrabutylammonium hydrogensulfate (316 mg, 0.930 mmol, 0.6 eq) and powdered dry sodium hydroxide (3100 mg, 77.5 mmol, 50 eq) was added forming a biphasic mixture. The reaction was cooled to −10° C. and o-toluoyl chloride (719 mg, 4.65 mmol, 3 eq) was added in a dropwise manner such that the reaction temperature did not exceed −5° C. After stirring at −5° C. for 1 h, the reaction mixture was filtered over celite and extracted with a dichloromethane (2×20 ml). The organic phase was washed with water (2×15 ml) and brine (1×15 ml) and then dried over magnesium sulphate before concentrating under vacuum. The resulting product was then purified by silica gel flash column chromatography using a dichloromethane/methanol gradient. There was obtained 561 mg (82%) of SYN-L-008 as a brown oil.

### Example 3—SYN-L-014 (1-cyclopropanesulfonyl-lyserqic acid diethylamide)

d-lysergic acid diethylamide (500 mg, 1.55 mmol, 1 eq) was dissolved in anhydrous tetrahydrofuran (20 ml) and cooled to −78° C. under a nitrogen atmosphere. With stirring, n-butyllithium (1.94 ml of 1.6 M in hexane, 3.10 mmol, 2 eq) was added in a dropwise manner such that the reaction temperature did not exceed −70° C. After stirring for 45 min, cyclopropylsulfonyl chloride (436 mg, 3.10 mmol, 2 eq) was then added in a dropwise manner such that the reaction temperature did not exceed −70° C. The resulting mixture was stirred at −78° C. overnight before careful addition of water (15 ml). The aqueous phase was extracted with dichloromethane (3×30 ml) and the organic phase washed with water (2×15 ml) and brine (1×15 ml) and then dried over magnesium sulphate before concentrating under vacuum. The resulting residue was then purified by silica gel flash column chromatography using a dichloromethane/methanol gradient. There was obtained 79 mg (12%) of SYN-L-014 as a brown solid.

### Example 4—SYN-L-015 (1-methoxyacetyl-lyserqic acid diethylamide)

d-lysergic acid diethylamide (500 mg, 1.55 mmol, 1 eq) was dissolved in anhydrous tetrahydrofuran (20 ml) and cooled to −78° C. under a nitrogen atmosphere. With stirring, n-butyllithium (1.94 ml of 1.6 M in hexane, 3.10 mmol, 2 eq) was added in a dropwise manner such that the reaction temperature did not exceed −70° C. After stirring for 60 min, methoxyacetyl chloride (336 mg, 3.10 mmol, 2 eq) was then added in a dropwise manner such that the reaction temperature did not exceed −70° C. The resulting mixture was stirred at −78° C. overnight before careful addition of water (15 ml). The aqueous phase was extracted with dichloromethane (3×30 ml) and the organic phase washed with water (2×15 ml) and brine (1×15 ml) and then dried over magnesium sulphate before concentrating under vacuum. The resulting residue was then purified by silica gel flash column chromatography using a dichloromethane/methanol gradient. There was obtained 49 mg (8%) of SYN-L-015 as a brown oil.

### Example 5—SYN-L-013 (1-enacarbil-lyserqic acid diethylamide)

d-lysergic acid diethylamide (500 mg, 1.55 mmol, 1 eq) was dissolved in anhydrous tetrahydrofuran (20 ml) and cooled to −78° C. under a nitrogen atmosphere. With stirring, n-butyllithium (1.94 ml of 1.6 M in hexane, 3.10 mmol, 2 eq) was added in a dropwise manner such that the reaction temperature did not exceed −70° C. After stirring for 30 min, 1-chloroethyl chloroformate (443 mg, 3.10 mmol, 2 eq) was then added in a dropwise manner such that the reaction temperature did not exceed −70° C. The resulting mixture was stirred at −78° C. overnight before careful addition of water (15 ml). The aqueous phase was extracted with dichloromethane (3×30 ml) and the organic phase washed with water (2×15 ml) and brine (1×15 ml) and then dried over magnesium sulphate before concentrating under vacuum. The resulting residue was then dissolved in N,N-dimethylformamide (5 ml). This was then added to a flask containing cesium isobutyrate (1360 mg, 6.20 mmol, 4 eq) dissolved in N,N-dimethylformamide (5 ml) under a nitrogen atmosphere and with stirring. After heating for 1 hr at 60° C., the orange solution was allowed to cool to room temperature before diluting with ethyl acetate (40 ml). The organic phase was washed with saturated aqueous ammonium chloride solution (3×15 ml) and brine (1×15 ml) and then dried over magnesium sulphate before concentrating under vacuum. The resulting residue was then purified by silica gel flash column chromatography using a toluene/acetone gradient. There was obtained 67 mg (9%) of SYN-L-013 as a brown oil.

### Example 6—SYN-L-012 (1-(methylsuccinyl-lyserqic acid diethylamide)

d-lysergic acid diethylamide (500 mg, 1.55 mmol, 1 eq) was dissolved in anhydrous acetonitrile (30 ml) at room temperature and under a nitrogen atmosphere. With stirring, succinylimidazole-monomethyl ester (339 mg, 1.86 mmol, 1.2 eq) and DBU (118 mg, 0.775 mmol, 0.5 eq) was added forming a dark solution. The reaction was heated at 40° C. for 1 h and then concentrated under vacuum. The residue was dissolved in dichloromethane (90 ml), washed with saturated sodium bicarbonate solution (1×15 ml), water (1×15 ml) and brine (1×15 ml) and then dried over magnesium sulphate before concentrating under vacuum. The resulting residue was then purified by silica gel flash column chromatography using a dichloromethane/methanol gradient. There was obtained 529 mg (78%) of SYN-L-012 as a brown foam.

### Example 7—SYN-L-068 (1-(2-furoyl-lyserqic acid 2,4-dimethylazetidide)

d-lysergic acid-2,4-dimethylazetidine (500 mg, 1.49 mmol, 1 eq) was dissolved in anhydrous acetonitrile (30 ml) at room temperature and under a nitrogen atmosphere. With stirring, 1-(2-furoyl)-imidazole (266 mg, 1.64 mmol, 1.1 eq) and DBU (113 mg, 0.745 mmol, 0.5 eq) was added forming a dark solution. The reaction was heated at 50° C. for 3 h and then concentrated under vacuum. The residue was dissolved in dichloromethane (90 ml), washed with saturated sodium bicarbonate solution (1×15 ml), water (1×15 ml) and brine (1×15 ml) and then dried over magnesium sulphate before concentrating under vacuum. The resulting residue was then purified by silica gel flash column chromatography using a dichloromethane/methanol gradient. There was obtained 454 mg (73%) of SYN-L-068 as a brown foam.

### Example 8—SYN-L-205 (1-(2-thiophenecarbonyl)-6-ethyl-6-nor-lyserqic acid methylisopropylamide)

Step 1: 1-propionyl-lysergic acid methylisopropylamide (500 mg, 1.32 mmol, 1 eq) was dissolved in chloroform (40 ml) at room temperature and under a nitrogen atmosphere. With stirring, cyanogen bromide (560 mg, 5.28 mmol, 4 eq) was carefully added forming a dark mixture. The reaction was heated at reflux for 8 h before cooling to room temperature and filtering over celite. The celite was extracted with further portions of chloroform (2×20 ml) and the combined organics were concentrated under vacuum to dryness. There was obtained 1-propionyl-6-cyano-6-nor-lysergic acid methylisopropylamide as a crude solid, which was used directly in the next step.

Step 2: The entire crude mass of 1-propionyl-6-cyano-6-nor-lysergic acid methylisopropylamide from step 1 was dissolved in glacial acetic acid (10 ml) at room temperature under a nitrogen atmosphere. With stirring, water (2 ml) and finely powder zinc (860 mg) was carefully added forming a suspension. The reaction was heated at reflux for 4 h before cooling to room temperature. With continued stirring, the reaction mixture was diluted with water (10 ml) and chilled before a portion of dichloromethane (30 ml) was added. Concentrated aqueous ammonia was added in a dropwise manner until a pH of 9 to 10 was maintained. The phases were allowed to separate and the aqueous layer was extracted with further portions of dichloromethane (2×20 ml). The combined organics were washed with water (2×15 ml) and brine (1×15 ml) and then dried over magnesium sulphate before concentrating under vacuum. There was obtained 1-propionyl-6-nor-lysergic acid methylisopropylamide as a crude residue, which was used directly in the next step.

Step 3: The entire crude mass of 1-propionyl-6-nor-lysergic acid methylisopropylamide from step 2 was dissolved in anhydrous acetonitrile (30 ml) at room temperature under a nitrogen atmosphere. Anhydrous potassium carbonate (900 mg) and ethyl iodide (220 mg) was added forming a suspension. This was heated at 50° C. for 3 h before concentrating under hard vacuum. The residue was taken up in dichloromethane (50 ml), filtered over celite and the organics dried over magnesium sulphate before concentrating under vacuum. The resulting residue, was then purified by silica gel flash column chromatography using a dichloromethane/methanol gradient. There was obtained 156 mg (30% over 3 steps) of 1-propionyl-6-ethyl-6-nor-lysergic acid methylisopropylamide as a brown foam.

Step 4: 1-propionyl-6-ethyl-6-nor-lysergic acid methylisopropylamide (150 mg, 0.381 mmol, 1 eq) was dissolved in methanol (20 ml) at room temperature under a nitrogen atmosphere. With stirring, concentrated aqueous ammonia (10 ml) was added and the homogenous mixture heated at 35° C. for 12 h. After cooling to room temperature, the methanol was removed under vacuum effectuating the precipitation of a gummy residue. The mixture was taken up in dichloromethane (30 ml) and the phases allowed to separate. The aqueous phase is extracted with further portions of dichloromethane (2×10 ml) and the combined organics are dried over magnesium sulphate before concentrating under vacuum. The resulting residue, was then purified by silica gel flash column chromatography using a dichloromethane/methanol gradient. There was obtained 105 mg (82%) of 6-ethyl-6-nor-lysergic acid methylisopropylamide as a beige foam. This was then acylated with 1-(2-thiophenecarbonyl)-imidazole using General Method C, as described by Scheme 3 to obtain 100 mg (72%) of SYN-L-205 as a brown solid.

### Example 9—SYN-L-143 (1-(2-furoyl)-6-allyl-6-nor-lyserqic acid diethylamide)

Step 1: 1-propionyl-lysergic acid lysergic acid diethylamide (500 mg, 1.32 mmol, 1 eq) was dissolved in chloroform (40 ml) at room temperature and under a nitrogen atmosphere. With stirring, cyanogen bromide (560 mg, 5.28 mmol, 4 eq) was carefully added forming a dark mixture. The reaction was heated at reflux for 6 h before cooling to room temperature and filtering over celite. The celite was extracted with further portions of chloroform (2×20 ml) and the combined organics were concentrated under vacuum to dryness. There was obtained 1-propionyl-6-cyano-6-nor-lysergic acid diethylamide as a crude solid, which was used directly in the next step.

Step 2: The entire crude mass of 1-propionyl-6-cyano-6-nor-lysergic acid diethylamide from step 1 was dissolved in glacial acetic acid (10 ml) at room temperature under a nitrogen atmosphere. With stirring, water (2 ml) and finely powder zinc (860 mg) was carefully added forming a suspension. The reaction was heated at reflux for 5 h before cooling to room temperature. With continued stirring, the reaction mixture was diluted with water (10 ml) and chilled before a portion of dichloromethane (30 ml) was added. Concentrated aqueous ammonia was added in a dropwise manner until a pH of 9 to 10 was maintained. The phases were allowed to separate and the aqueous layer was extracted with further portions of dichloromethane (2×20 ml). The combined organics were washed with water (2×15 ml) and brine (1×15 ml) and then dried over magnesium sulphate before concentrating under vacuum. There was obtained 1-propionyl-6-nor-lysergic acid diethylamide as a crude residue, which was used directly in the next step.

Step 3: The entire crude mass of 1-propionyl-6-nor-lysergic acid diethylamide from step 2 was dissolved in anhydrous acetonitrile (30 ml) at room temperature under a nitrogen atmosphere. Anhydrous potassium carbonate (900 mg) and allyl bromide (160 mg) was added forming a suspension. This was heated at 40° C. for 6 h before concentrating under hard vacuum. The residue was taken up in dichloromethane (50 ml), filtered over celite and the organics dried over magnesium sulphate before concentrating under vacuum. The resulting residue, was then purified by silica gel flash column chromatography using a dichloromethane/methanol gradient. There was obtained 203 mg (38% over 3 steps) of 1-propionyl-6-allyl-6-nor-lysergic acid diethylamide as a brown foam.

Step 4: 1-propionyl-6-allyl-6-nor-lysergic acid diethylamide (150 mg, 0.370 mmol, 1 eq) was dissolved in methanol (20 ml) at room temperature under a nitrogen atmosphere. With stirring, concentrated aqueous ammonia (10 ml) was added and the homogenous mixture heated at 45° C. for 8 h. After cooling to room temperature, the methanol was removed under vacuum effectuating the precipitation of a gummy residue. The mixture was taken up in dichloromethane (30 ml) and the phases allowed to separate. The aqueous phase is extracted with further portions of dichloromethane (2×10 ml) and the combined organics are dried over magnesium sulphate before concentrating under vacuum. The resulting residue, was then purified by silica gel flash column chromatography using a dichloromethane/methanol gradient. There was obtained 110 mg (85%) of 6-allyl-6-nor-lysergic acid diethylamide as a beige foam. This was then acylated with 1-(2-furoyl)-imidazole using General Method C, as described by Scheme 3 to obtain 95 mg (68%) of SYN-L-143 as a brown solid.

Example 10—SYN-L-017 (1-propionyl-2-bromo-lyserqic acid diethylamide) Step 1: Tetrabutylammonium hydroxide aqueous solution (13.8 g, 53.2 mmol, 4 eq), sodium hydroxide (3.19 g, 79.8 mmol, 6 eq), sodium dithionite (0.463 g, 2.66 mmol, 0.2 eq) were dissolved in water (80 ml) at room temperature under a nitrogen atmosphere. With stirring, bromocriptine mesylate (10.0 g, 13.3 mmol, 1 eq) was added in small portions forming a mixture. The reaction was heated to reflux for 8 h before cooling to room temperature. The resulting dark solution was acidified with acetic acid until a pH of 6 to 6.5 was maintained. With continued stirring, the reaction was cooled to 5° C. for 8 h, effectuating precipitation. The mixture was filtered under vacuum and the filter cake washed with several portions of water (3×60 ml) and then isopropanol (1×60 ml). The filter cake was dried in the oven at 80 C for 3 days under hard vacuum. There was obtained 3.00 g (65%) of 2-bromo-lysergic acid as a grey solid, which didn't require additional purification and was used directly in the next step.

Step 2: 2-bromo-lysergic acid (1.00 g, 2.88 mmol, 1 eq) was suspended in chloroform (25 ml) at room temperature under a nitrogen atmosphere. With stirring, diethylamine (1.90 g, 25.9 mmol, 9 eq) was added in one portion. The reaction was heated to 40 C and phosphorus oxychloride (0.883 g, 5.76 mmol, 2 eq) was added in a dropwise manner such that reflux was maintained. After the addition, the reaction was stirred for an additional 30 min. With continued stirring, water (10 ml) and then concentrated aqueous ammonia was added such that the mixture was sufficiently basic. The layers were allowed to separate and the aqueous phase was extracted with dichloromethane (2×30 ml). The combined organics were washed with water (2×15 ml) and brine (1×15 ml) and then dried over magnesium sulphate before concentrating under vacuum. The resulting residue, was then purified by silica gel flash column chromatography using a dichloromethane/methanol gradient. There was obtained 658 mg (57%) of 2-bromo-lysergic acid diethylamide as a light brown foam. This was then acylated with propionyl chloride using General Method A, as described by Scheme 1 to obtain 448 mg (60%) of SYN-L-017 as a brown oil.

### Example 11—SYN-L-206 (1-propionyl-2-methyl-lyserqic acid diethylamide)

2-bromo-lysergic acid diethylamide (200 mg, 0.499 mmol, 1 eq) and [1,1′-Bis(diphenylphosphino)ferrocene]dichloropalladium(II) (18.3 mg, 0.0250 mmol, 0.05 eq) was dissolved in anhydrous 1,4-dioxane (8 ml) at room temperature under a nitrogen atmosphere. Dimethyl zinc solution (95.3 mg, 0.998 mmol, 2 eq) was carefully added and the reaction heated to reflux for 90 min. After cooling to room temperature, the reaction was quenched with methanol (8 ml) and concentrated under vacuum. The resulting residue, was then purified directly by silica gel flash column chromatography using an ethyl acetate/isopropanol gradient. There was obtained 71 mg (42%) of 2-methyl-lysergic acid diethylamide as a brown oil. This was then acylated with propionyl chloride using General Method A, as described by Scheme 1 to obtain 56 mg (68%) of SYN-L-206 as a brown oil.

### Example 12—SYN-L-212 (1-propionyl-9,10-dihydro-lyserqic acid diethylamide)

d-lysergic acid diethylamide (500 mg, 1.55 mmol, 1 eq) was dissolved in anhydrous methanol (20 ml) at 35° C. With stirring, palladium (125 mg, 10% on carbon) suspended in methanol (5 ml) was added and the reaction mixture was purged with nitrogen. The reaction was then purged with hydrogen and put under a hydrogen balloon at 35° C. for 5 h. After cooling to room temperature, the reaction mixture was filter over celite and extracted with methanol (2×20 ml). The combined organics were concentrated under vacuum and the resulting residue was purified directly by silica gel flash column chromatography using a dichloromethane/methanol gradient. There was obtained 408 mg (81%) of 9,10-dihydro-lysergic acid diethylamide as a light foam. This was then acylated with 1-propionylimidazole using General Method C, as described by Scheme 3 to obtain 363 mg (76%) of SYN-L-212 as a light brown oil.

### Example 13—SYN-L-211 (1-propionyl-9,10-cyclopropyl-lyserqic acid diethylamide)

d-lysergic acid diethylamide (500 mg, 1.55 mmol, 1 eq) and palladium(II) acetate (69.6 mg, 0.310 mmol, 0.2 eq) was dissolved in dichloromethane (20 ml) at room temperature under a nitrogen atmosphere. With stirring and cooling to 0° C., diazomethane solution (1.30 g, 31.0 mmol, 20 eq) was added slowly in a dropwise manner. Stirring was continued at 0° C. for 6 h before filtering and concentrating the organics under vacuum. The resulting residue was purified directly by silica gel flash column chromatography using a dichloromethane/methanol gradient. There was obtained 94 mg (18%) of 9,10-cyclopropyl-lysergic acid diethylamide as a brown oil. This was then acylated with 1-propionylimidazole using General Method C, as described by Scheme 3 to obtain 78 mg (71%) of SYN-L-211 as a brown oil.

Further compounds of the invention were prepared following the teaching of the general methods and specific preparation examples, as reflected in the following analytical characterization and pharmaceutical testing, as a person skilled in the art would know how to modify the General Methods to accommodate the different substituents that may be present on the compounds of the invention.

### Analytical Characterisation, Compounds of the Invention, Characterisation Data:

¹H NMR for selected compounds SYN-L-004 to SYN-L-236 of the invention are shown in the following.

### SYN-L-003, 1-ethylcarbamoyl-lyserqic acid diethylamide

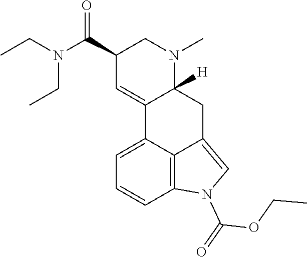 [SMILES: CCOC(=O)n1cc2c3c(cccc31)C1=C[C@@H](C(=O)N(CC)CC)CN(C)[C@@H]1C2]

1H NMR: δ 1.22 (6H, t), 1.30 (3H, t), 2.57 (3H, s), 2.83-3.06 (4H, m), 3.22-3.40 (5H, m), 4.03 (1H, dd), 4.13-4.25 (2H, m), 6.16 (1H, d), 6.91-7.17 (2H, m), 7.65 (1H, ddd), 7.73 (1H, d).

### SYN-L-004, 1-dodecanoyl-lyserqic acid diethylamide

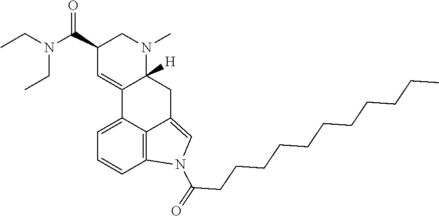 [SMILES: CCCCCCCCCCCC(=O)n1cc2c3c(cccc31)C1=C[C@@H](C(=O)N(CC)CC)CN(C)[C@@H]1C2]

1H NMR: δ 0.86 (3H, t), 1.16-1.34 (22H, m), 1.47-1.59 (2H, m), 2.52-2.68 (5H, m), 2.82-3.07 (4H, m), 3.22-3.41 (5H, m), 3.95 (1H, dd), 6.15 (1H, d), 6.87-7.12 (2H, m), 7.53 (1H, ddd), 7.93 (1H, d).

### SYN-L-005, 1-(2-furoyl)-lyserqic acid diethylamide

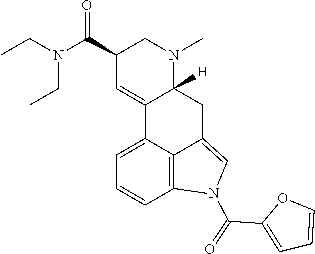 [SMILES: CCN(CC)C(=O)[C@@H]1C=C2c3cccc4c3c(cn4C(=O)c3ccco3)C[C@H]2N(C)C1]

1H NMR: δ 1.22 (6H, t), 2.57 (3H, s), 2.83-3.06 (4H, m), 3.22-3.41 (5H, m), 4.04 (1H, dd), 6.15 (1H, d), 6.65 (1H, dd), 6.88-7.13 (2H, m), 7.30 (1H, dd), 7.56 (1H, m), 7.84 (1H, dd), 8.00 (1H, d).

### SYN-L-006, 1-(3-phenylpropanoyl)-lyserqic acid diethylamide

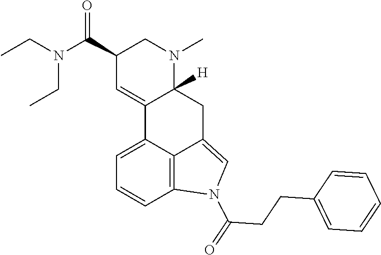 [SMILES: CCN(CC)C(=O)[C@@H]1C=C2c3cccc4c3c(cn4C(=O)CCc3ccccc3)C[C@H]2N(C)C1]

1H NMR: δ 1.22 (6H, t), 2.57 (3H, s), 2.73-3.08 (8H, m), 3.22-3.41 (5H, m), 3.95 (1H, dd), 6.15 (1H, d), 6.87-7.34 (7H, m), 7.53 (1H, m), 7.93 (1H, d).

### SYN-L-008, 1-(o-toluoyl)-lyserqic acid diethylamide

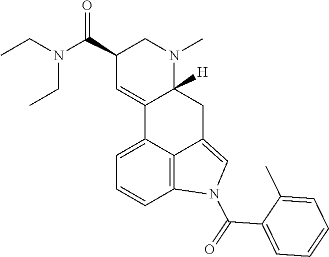 [SMILES: CCN(CC)C(=O)[C@@H]1C=C2c3cccc4c3c(cn4C(=O)c3ccccc3C)C[C@H]2N(C)C1]

1H NMR: δ 1.22 (6H, t), 2.40 (3H, s), 2.57 (3H, s), 2.82-3.07 (4H, m), 3.22-3.41 (5H, m), 4.04 (1H, dd), 6.15 (1H, d), 6.87-7.23 (3H, m), 7.38-7.72 (4H, m), 7.97 (1H, d).

### SYN-L-010, 1-nicotinoyl-lyserqic acid diethylamide

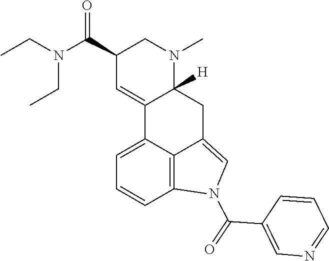 [SMILES: CCN(CC)C(=O)[C@@H]1C=C2c3cccc4c3c(cn4C(=O)c3cccnc3)C[C@H]2N(C)C1]

1H NMR: δ 1.22 (6H, t), 2.57 (3H, s), 2.82-3.07 (4H, m), 3.22-3.41 (5H, m), 3.99 (1H, dd), 6.15 (1H, d), 6.88-7.13 (2H, m), 7.47-7.61 (2H, m), 7.94 (1H, d), 8.07 (1H, ddd), 8.63 (1H, dt), 9.08 (1H, ddd).

### SYN-L-011, 1-succinyl-lyserqic acid diethylamide

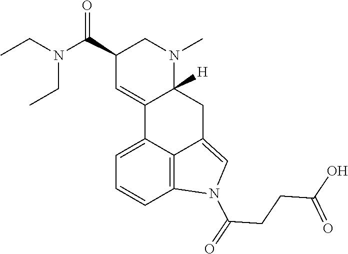 [SMILES: CCN(CC)C(=O)[C@@H]1C=C2c3cccc4c3c(cn4C(=O)CCC(=O)O)C[C@H]2N(C)C1]

1H NMR: δ 1.22 (6H, t), 2.57 (3H, s), 2.79-3.07 (8H, m), 3.22-3.41 (5H, m), 3.99 (1H, dd), 6.15 (1H, d), 6.87-7.12 (2H, m), 7.53 (1H, ddd), 7.93 (1H, d).

### SYN-L-012, 1-methylsuccinyl-lyserqic acid diethylamide

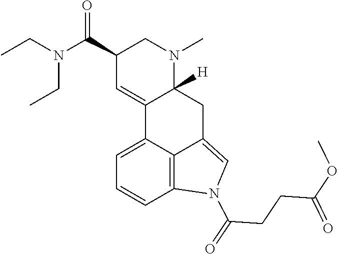 [SMILES: CCN(CC)C(=O)[C@@H]1C=C2c3cccc4c3c(cn4C(=O)CCC(=O)OC)C[C@H]2N(C)C1]

1H NMR: δ 1.22 (6H, t), 2.57 (3H, s), 2.69-2.81 (2H, m), 2.82-3.07 (6H, m), 3.22-3.41 (5H, m), 3.64 (3H, s), 3.99 (1H, dd), 6.15 (1H, d), 6.87-7.12 (2H, m), 7.53 (1H, ddd), 7.93 (1H, d).

### SYN-L-013, 1-enacarbil-lyserqic acid diethylamide

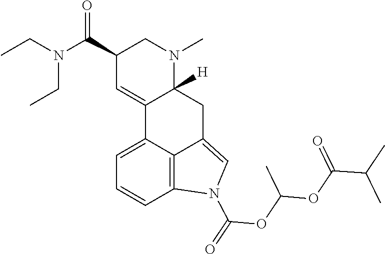 [SMILES: CCN(CC)C(=O)[C@@H]1C=C2c3cccc4c3c(cn4C(=O)OC(C)OC(=O)C(C)C)C[C@H]2N(C)C1]

1H NMR: δ 1.06-1.28 (12H, m), 1.61 (3H, d), 2.42 (1H, m), 2.57 (3H, s), 2.82-3.06 (4H, m), 3.22-3.42 (5H, m), 3.98 (1H, dd), 6.01-6.21 (2H, m), 6.91-7.18 (2H, m), 7.65 (1H, ddd), 7.75 (1H, d).

### SYN-L-014, 1-cyclopropanesulfonyl-lyserqic acid diethylamide

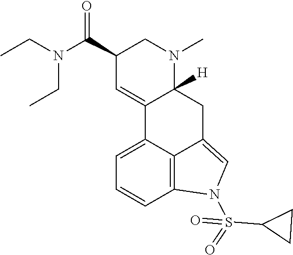 [SMILES: CCN(CC)C(=O)[C@@H]1C=C2c3cccc4c3c(cn4S(=O)(=O)C3CC3)C[C@H]2N(C)C1]

1H NMR: δ 1.16-1.39 (10H, m), 2.57 (3H, s), 2.92-3.17 (4H, m), 3.18-3.40 (6H, m), 4.40 (1H, dd), 6.19 (1H, d), 7.11-7.46 (3H, m), 8.30 (1H, d).

### SYN-L-015, 1-methoxyacetyl-lyserqic acid diethylamide

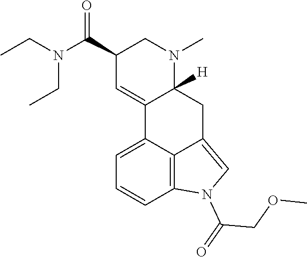 [SMILES: CCN(CC)C(=O)[C@@H]1C=C2c3cccc4c3c(cn4C(=O)COC)C[C@H]2N(C)C1]

1H NMR: δ 1.22 (6H, t), 2.57 (3H, s), 2.81-3.07 (4H, m), 3.16-3.41 (8H, m), 3.99 (1H, dd), 4.47-4.57 (2H, m), 6.15 (1H, d), 6.88-7.13 (2H, m), 7.55 (1H, ddd), 8.01 (1H, d).

### SYN-L-017, 1-propionyl-2-bromo-lyserqic acid diethylamide

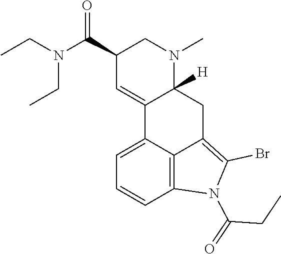 [SMILES: CCC(=O)n1c(Br)c2c3c(cccc31)C1=C[C@@H](C(=O)N(CC)CC)CN(C)[C@@H]1C2]

1H NMR: δ 1.06 (3H, t), 1.22 (6H, t), 2.57 (3H, s), 2.63-2.75 (2H, m), 2.92-3.12 (4H, m), 3.22-3.38 (5H, m), 4.03 (1H, dd), 6.13 (1H, d), 6.87-7.09 (2H, m), 7.27 (1H, dd).

### SYN-L-018, 1-benzoyl-lyserqic acid diethylamide

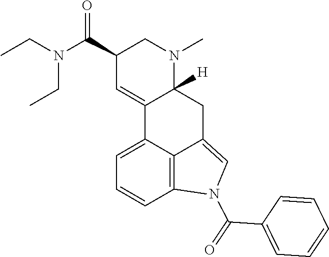 [SMILES: CCN(CC)C(=O)[C@@H]1C=C2c3cccc4c3c(cn4C(=O)c3ccccc3)C[C@H]2N(C)C1]

1H NMR: δ 1.22 (6H, t), 2.57 (3H, s), 2.82-3.07 (4H, m), 3.22-3.41 (5H, m), 3.99 (1H, dd), 6.15 (1H, d), 6.87-7.13 (2H, m), 7.39-7.75 (6H, m), 7.93 (1H, d).

### SYN-L-021, 1-(2-thiophenecarbonyl)-lyserqic acid diethylamide

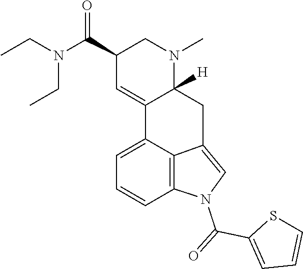 [SMILES: CCN(CC)C(=O)[C@@H]1C=C2c3cccc4c3c(cn4C(=O)c3cccs3)C[C@H]2N(C)C1]

1H NMR: δ 1.22 (6H, t), 2.57 (3H, s), 2.82-3.07 (4H, m), 3.22-3.41 (5H, m), 4.04 (1H, dd), 6.15 (1H, d), 6.88-7.13 (2H, m), 7.20 (1H, dd), 7.59 (1H, ddd), 7.77 (1H, dd), 7.96 (1H, dd), 7.98 (1H, d).

### SYN-L-022, 1-phenylacetyl-lyserqic acid diethylamide

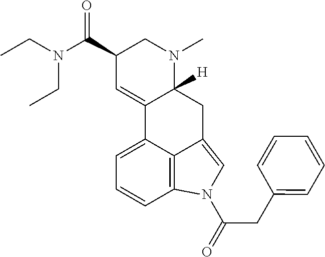 [SMILES: CCN(CC)C(=O)[C@@H]1C=C2c3cccc4c3c(cn4C(=O)Cc3ccccc3)C[C@H]2N(C)C1]

1H NMR: δ 1.22 (6H, t), 2.57 (3H, s), 2.82-3.07 (4H, m), 3.22-3.41 (5H, m), 3.88-4.12 (3H, m), 6.15 (1H, d), 6.87-7.12 (2H, m), 7.21-7.45 (5H, m), 7.54 (1H, ddd), 7.98 (1H, d).

### SYN-L-023, 1-(1-adamantanecarbonyl)-lyserqic acid diethylamide

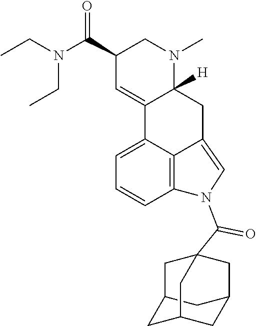 [SMILES: CCN(CC)C(=O)[C@@H]1C=C2c3cccc4c3c(cn4C(=O)C34CC5CC(CC(C5)C3)C4)C[C@H]2N(C)C1]

1H NMR: δ 1.22 (6H, t), 1.58-1.73 (6H, m), 1.77-1.94 (9H, m), 2.57 (3H, s), 2.81-3.07 (4H, m), 3.22-3.40 (5H, m), 3.99 (1H, dd), 6.16 (1H, d), 6.88-7.13 (2H, m), 7.53 (1H, ddd), 8.00 (1H, d).

### SYN-L-024, 1-pivaloyl-lyserqic acid diethylamide

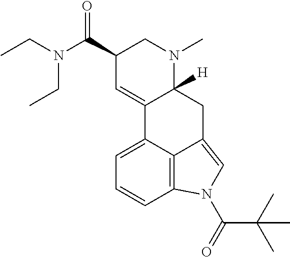 [SMILES: CCN(CC)C(=O)[C@@H]1C=C2c3cccc4c3c(cn4C(=O)C(C)(C)C)C[C@H]2N(C)C1]

1H NMR: δ 1.16-1.28 (15H, m), 2.57 (3H, s), 2.82-3.07 (4H, m), 3.22-3.41 (5H, m), 3.95 (1H, dd), 6.15 (1H, d), 6.88-7.13 (2H, m), 7.54 (1H, ddd), 7.98 (1H, d).

### SYN-L-026, 1-isovaleryl-lyserqic acid diethylamide

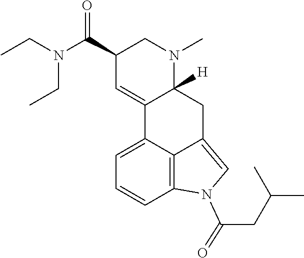 [SMILES: CCN(CC)C(=O)[C@@H]1C=C2c3cccc4c3c(cn4C(=O)CC(C)C)C[C@H]2N(C)C1]

1H NMR: δ 0.81-0.93 (6H, m), 1.22 (6H, t), 1.92 (1H, m), 2.50-2.62 (5H, m), 2.81-3.06 (4H, m), 3.22-3.40 (5H, m), 4.00 (1H, dd), 6.15 (1H, d), 6.87-7.12 (2H, m), 7.24 (1H, ddd), 7.93 (1H, d).

### SYN-L-027, 1-hexanoyl-lyserqic acid diethylamide

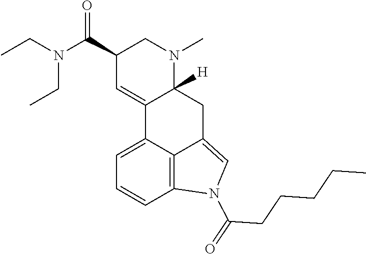 [SMILES: CCCCCC(=O)n1cc2c3c(cccc31)C1=C[C@@H](C(=O)N(CC)CC)CN(C)[C@@H]1C2]

1H NMR: δ 0.87 (3H, t), 1.16-1.35 (10H, m), 1.46-1.58 (2H, m), 2.52-2.68 (5H, m), 2.82-3.07 (4H, m), 3.22-3.41 (5H, m), 3.99 (1H, dd), 6.15 (1H, d), 6.87-7.12 (2H, m), 7.53 (1H, ddd), 7.93 (1H, d).

### SYN-L-028, 1-cyclopentanepropanoyl-lyserqic acid diethylamide

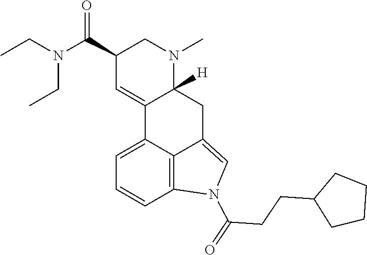 [SMILES: CCN(CC)C(=O)[C@@H]1C=C2c3cccc4c3c(cn4C(=O)CCC3CCCC3)C[C@H]2N(C)C1]

¹H NMR: δ 1.22 (6H, t), 1.35-1.78 (10H), 1.96 (1H, m), 2.52-2.70 (5H, m), 2.81-3.06 (4H, m), 3.22-3.40 (5H, m), 4.00 (1H, dd), 6.15 (1H, d), 6.87-7.12 (2H, m), 7.24 (1H, ddd), 7.93 (1H, d).

### SYN-L-029, 1-cyclohexanecarbonyl-lyserqic acid diethylamide

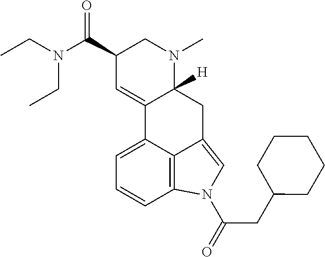 [SMILES: CCN(CC)C(=O)[C@@H]1C=C2c3cccc4c3c(cn4C(=O)CC3CCCCC3)C[C@H]2N(C)C1]

1H NMR: δ 1.22 (6H, t), 1.29-1.70 (6H, m), 1.76-2.06 (4H, m), 2.57 (3H, s), 2.81-3.06 (4H, m), 3.22-3.40 (6H, m), 4.00 (1H, dd), 6.16 (1H, d), 6.87-7.13 (2H, m), 7.53 (1H, ddd), 7.98 (1H, d).

### SYN-L-030, 1-cyclopentanecarbonyl-lyserqic acid diethylamide

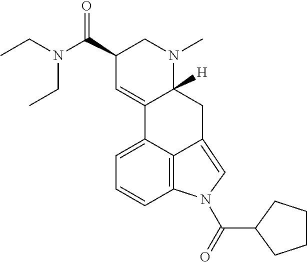 [SMILES: CCN(CC)C(=O)[C@@H]1C=C2c3cccc4c3c(cn4C(=O)C3CCCC3)C[C@H]2N(C)C1]

1H NMR: δ 1.22 (6H, t), 1.60-2.18 (8H, m), 2.57 (3H, s), 2.81-3.06 (4H, m), 3.22-3.50 (6H, m), 4.00 (1H, dd), 6.16 (1H, d), 6.87-7.12 (2H, m), 7.52 (1H, ddd), 7.98 (1H, d).

### SYN-L-031, 1-cyclobutanecarbonyl-lyserqic acid diethylamide

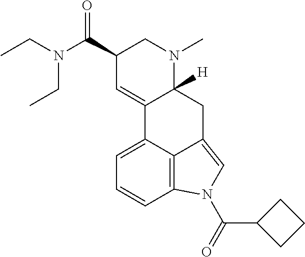 [SMILES: CCN(CC)C(=O)[C@@H]1C=C2c3cccc4c3c(cn4C(=O)C3CCC3)C[C@H]2N(C)C1]

1H NMR: δ 1.22 (6H, t), 1.80-2.03 (2H, m), 2.21-2.47 (4H, m), 2.57 (3H, s), 2.82-3.07 (4H, m), 3.18-3.43 (6H, m), 4.02 (1H, dd), 6.15 (1H, d), 6.86-7.12 (2H, m), 7.24 (1H, ddd), 7.97 (1H, d).

### SYN-L-032, 1-isobutyryl-lyserqic acid diethylamide

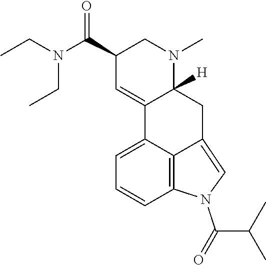 [SMILES: CCN(CC)C(=O)[C@@H]1C=C2c3cccc4c3c(cn4C(=O)C(C)C)C[C@H]2N(C)C1]

1H NMR: δ 1.07-1.28 (12H, m), 2.57 (3H, s), 2.75-3.07 (5H, m), 3.22-3.43 (5H, m), 4.02 (1H, dd), 6.15 (1H, d), 6.86-7.12 (2H, m), 7.24 (1H, ddd), 7.97 (1H, d).

### SYN-L-033, 1-(4-methylpentanoyl)-lyserqic acid diethylamide

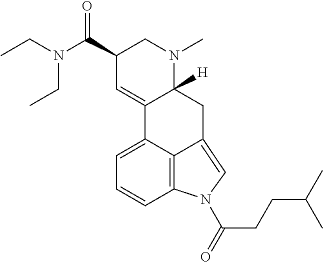 [SMILES: CCN(CC)C(=O)[C@@H]1C=C2c3cccc4c3c(cn4C(=O)CCC(C)C)C[C@H]2N(C)C1]

1H NMR: δ 0.83-0.94 (6H, m), 1.22 (6H, t), 1.36-1.55 (3H, m), 2.52-2.69 (5H, m), 2.82-3.07 (4H, m), 3.22-3.43 (5H, m), 4.02 (1H, dd), 6.15 (1H, d), 6.85-7.12 (2H, m), 7.23 (1H, ddd), 7.97 (1H, d).

### SYN-L-034, 1-(5-methyl-2-furoyl)-lyserqic acid diethylamide

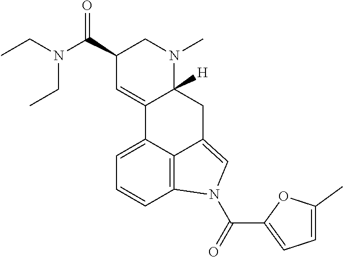 [SMILES: CCN(CC)C(=O)[C@@H]1C=C2c3cccc4c3c(cn4C(=O)c3ccc(C)o3)C[C@H]2N(C)C1]

¹H NMR: δ 1.22 (6H, t), 2.42-2.62 (6H, m), 2.82-3.07 (4H, m), 3.22-3.40 (5H, m), 4.04 (1H, dd), 6.15 (1H, d), 6.34 (1H, d), 6.88-7.13 (2H, m), 7.20 (1H, d), 7.54 (1H, ddd), 8.00 (1H, d).

### SYN-L-035, 1-(5-tert-butyl-2-furoyl)-lyserqic acid diethylamide

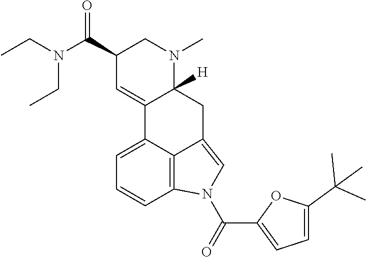 [SMILES: CCN(CC)C(=O)[C@@H]1C=C2c3cccc4c3c(cn4C(=O)c3ccc(C(C)(C)C)o3)C[C@H]2N(C)C1]

1H NMR: δ 1.16-1.29 (15H, m), 2.57 (3H, s), 2.82-3.07 (4H, m), 3.22-3.40 (5H, m), 4.04 (1H, dd), 6.15 (1H, d), 6.45 (1H, d), 6.88-7.13 (2H, m), 7.23 (1H, d), 7.54 (1H, ddd), 8.00 (1H, d).

### SYN-L-036, 1-(tetrahydro-2-furoyl)-lyserqic acid diethylamide

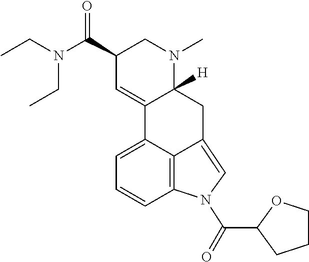 [SMILES: CCN(CC)C(=O)[C@@H]1C=C2c3cccc4c3c(cn4C(=O)C3CCCO3)C[C@H]2N(C)C1]

1H NMR: δ 1.22 (6H, t), 1.96-2.20 (4H, m), 2.57 (3H, s), 2.82-3.07 (4H, m), 3.22-3.41 (5H, m), 3.61-3.79 (2H, m), 3.95 (1H, dd), 5.25 (1H, dd), 6.15 (1H, d), 6.88-7.13 (2H, m), 7.55 (1H, ddd), 8.03 (1H, d).

### SYN-L-037, 1-(3-furoyl)-lyserqic acid diethylamide

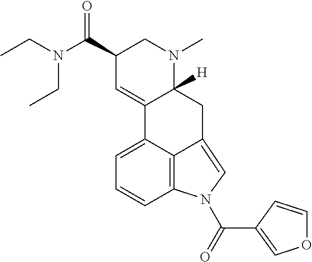 [SMILES: CCN(CC)C(=O)[C@@H]1C=C2c3cccc4c3c(cn4C(=O)c3ccoc3)C[C@H]2N(C)C1]

1H NMR: δ 1.22 (6H, t), 2.57 (3H, s), 2.81-3.07 (4H, m), 3.22-3.40 (5H, m), 4.04 (1H, dd), 6.15 (1H, d), 6.84-7.13 (3H, m), 7.47-7.74 (3H, m), 7.89 (1H, d).

### SYN-L-038, 1-(3-thiophenecarbonyl)-lyserqic acid diethylamide

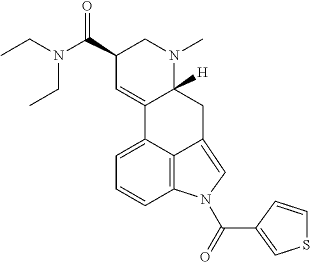 [SMILES: CCN(CC)C(=O)[C@@H]1C=C2c3cccc4c3c(cn4C(=O)c3ccsc3)C[C@H]2N(C)C1]

1H NMR: δ 1.22 (6H, t), 2.57 (3H, s), 2.81-3.07 (4H, m), 3.22-3.40 (5H, m), 4.04 (1H, dd), 6.15 (1H, d), 6.88-7.13 (2H, m), 7.54 (1H, ddd), 7.62-7.80 (2H, m), 7.97 (1H, d), 8.32 (1H, dd).

### SYN-L-039, 1-isonicotinoyl-lyserqic acid diethylamide

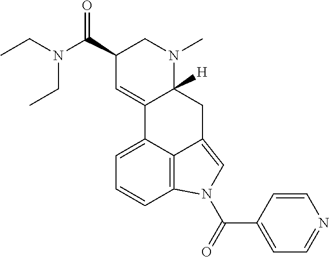 [SMILES: CCN(CC)C(=O)[C@@H]1C=C2c3cccc4c3c(cn4C(=O)c3ccncc3)C[C@H]2N(C)C1]

1H NMR: δ 1.22 (6H, t), 2.57 (3H, s), 2.81-3.07 (4H, m), 3.22-3.41 (5H, m), 4.01 (1H, dd), 6.15 (1H, d), 6.86-7.12 (2H, m), 7.26 (1H, ddd), 7.87-8.04 (3H, m), 8.71 (2H, ddd).

### SYN-L-040, 1-(3-oxetanoyl)-lyserqic acid diethylamide

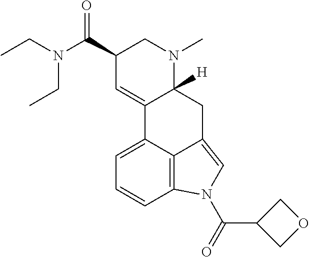 [SMILES: CCN(CC)C(=O)[C@@H]1C=C2c3cccc4c3c(cn4C(=O)C3COC3)C[C@H]2N(C)C1]

1H NMR: δ 1.22 (6H, t), 2.57 (3H, s), 2.82-3.07 (4H, m), 3.22-3.49 (6H, m), 3.88-4.09 (5H, m), 6.15 (1H, d), 6.86-7.12 (2H, m), 7.24 (1H, ddd), 7.99 (1H, d).

### SYN-L-041, 1-(4-tetrahydropyrancarbonyl)-lyserqic acid diethylamide

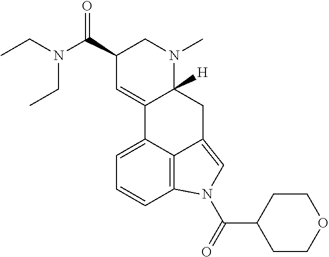 [SMILES: CCN(CC)C(=O)[C@@H]1C=C2c3cccc4c3c(cn4C(=O)C3CCOCC3)C[C@H]2N(C)C1]

1H NMR: δ 1.22 (6H, t), 1.75-1.92 (4H, m), 2.57 (3H, s), 2.81-3.07 (4H, m), 3.22-3.64 (10H, m), 4.02 (1H, dd), 6.15 (1H, d), 6.86-7.12 (2H, m), 7.24 (1H, ddd), 7.98 (1H, d).

### SYN-L-042, 1-stearoyl-lyserqic acid diethylamide

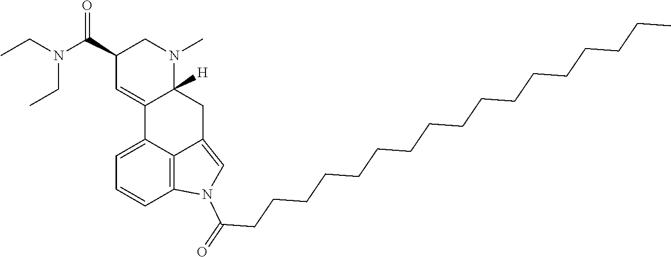 [SMILES: CCCCCCCCCCCCCCCCCC(=O)n1cc2c3c(cccc31)C1=C[C@@H](C(=O)N(CC)CC)CN(C)[C@@H]1C2]

1H NMR: δ 0.86 (3H, t), 1.16-1.34 (34H, m), 1.47-1.59 (2H, m), 2.52-2.68 (5H, m), 2.81-3.06 (4H, m), 3.22-3.40 (5H, m), 4.00 (1H, dd), 6.15 (1H, d), 6.87-7.12 (2H, m), 7.24 (1H, ddd), 7.93 (1H, d).

### SYN-L-043, 1-oleoyl-lyserqic acid diethylamide

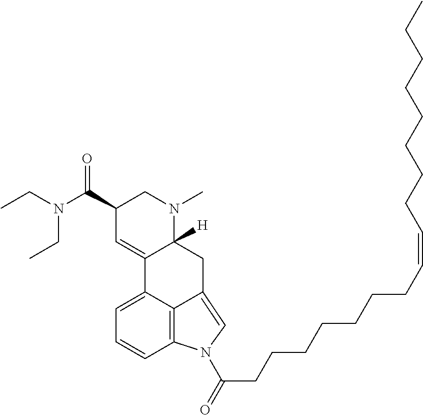 [SMILES: CCCCCCCC/C=C\CCCCCCCC(=O)n1cc2c3c(cccc31)C1=C[C@@H](C(=O)N(CC)CC)CN(C)[C@@H]1C2]

1H NMR: δ 0.86 (3H, t), 1.16-1.34 (22H, m), 1.35-1.59 (6H, m), 1.90-2.03 (4H, m), 2.52-2.68 (5H, m), 2.81-3.06 (4H, m), 3.22-3.40 (5H, m), 4.00 (1H, dd), 5.26-5.41 (2H, m), 6.15 (1H, d), 6.87-7.12 (2H, m), 7.24 (1H, ddd), 7.93 (1H, d).

### SYN-L-044, 1-arachidonoyl-lyserqic acid diethylamide

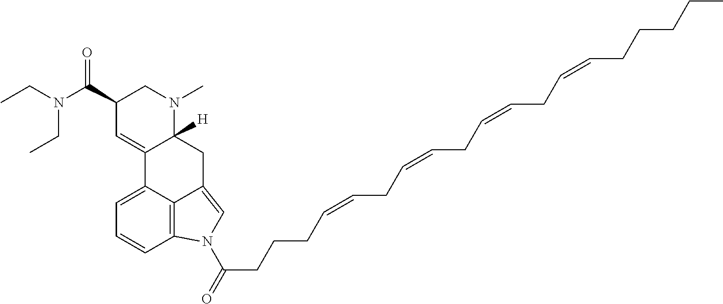 [SMILES: CCCCC/C=C\C/C=C\C/C=C\C/C=C\CCCC(=O)n1cc2c3c(cccc31)C1=C[C@@H](C(=O)N(CC)CC)CN(C)[C@@H]1C2]

1H NMR: δ 0.86 (3H, t), 1.16-1.34 (10H, m), 1.35-1.49 (2H, m), 1.67-1.79 (2H, m), 1.90-2.06 (4H, m), 2.52-2.71 (11H, m), 2.81-3.06 (4H, m), 3.22-3.40 (5H, m), 4.00 (1H, dd), 5.28-5.45 (8H, m), 6.15 (1H, d), 6.87-7.12 (2H, m), 7.24 (1H, ddd), 7.93 (1H, d).

### SYN-L-045, 1-linoleoyl-lyserqic acid diethylamide

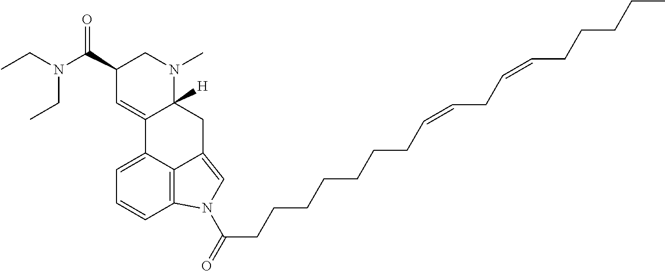 [SMILES: CCCCC/C=C\C/C=C\CCCCCCCC(=O)n1cc2c3c(cccc31)C1=C[C@@H](C(=O)N(CC)CC)CN(C)[C@@H]1C2]

1H NMR: δ 0.86 (3H, t), 1.16-1.34 (16H, m), 1.35-1.59 (6H, m), 1.90-2.04 (4H, m), 2.52-2.69 (7H, m), 2.81-3.06 (4H, m), 3.22-3.40 (5H, m), 4.00 (1H, dd), 5.28-5.44 (4H, m), 6.15 (1H, d), 6.87-7.12 (2H, m), 7.24 (1H, ddd), 7.93 (1H, d).

### SYN-L-046, 1-(4-morpholinylbutyryl)-lyserqic acid diethylamide

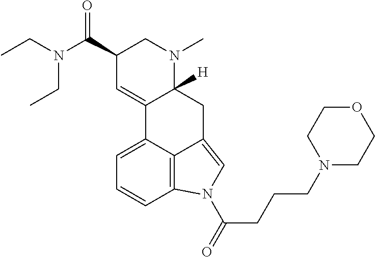 [SMILES: CCN(CC)C(=O)[C@@H]1C=C2c3cccc4c3c(cn4C(=O)CCCN3CCOCC3)C[C@H]2N(C)C1]

1H NMR: δ 1.22 (6H, t), 1.91-2.03 (2H, m), 2.40-2.72 (11H, m), 2.81-3.06 (4H, m), 3.22-3.40 (5H, m), 3.52-3.67 (4H, m), 4.00 (1H, dd), 6.15 (1H, d), 6.87-7.12 (2H, m), 7.24 (1H, ddd), 7.93 (1H, d).

### SYN-L-047, 1-(4-morpholinecarbonyl)-lyserqic acid diethylamide

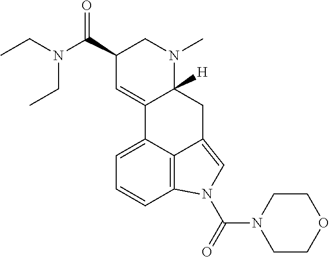 [SMILES: CCN(CC)C(=O)[C@@H]1C=C2c3cccc4c3c(cn4C(=O)N3CCOCC3)C[C@H]2N(C)C1]

1H NMR: δ 1.22 (6H, t), 2.57 (3H, s), 2.82-3.07 (4H, m), 3.22-3.40 (5H, m), 3.48-3.76 (8H, m), 4.03 (1H, dd), 6.15 (1H, d), 6.88-7.13 (2H, m), 7.55 (1H, ddd), 7.84 (1H, d).

### SYN-L-048, 1-(p-tosyl)-lyserqic acid diethylamide

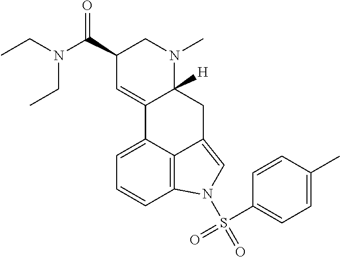 [SMILES: CCN(CC)C(=O)[C@@H]1C=C2c3cccc4c3c(cn4S(=O)(=O)c3ccc(C)cc3)C[C@H]2N(C)C1]

1H NMR: δ 1.22 (6H, t), 2.42-2.62 (6H, m), 2.89-3.15 (4H, m), 3.22-3.44 (5H, m), 4.37 (1H, dd), 6.19 (1H, d), 7.08-7.49 (5H, m), 7.58 (2H, ddd), 8.27 (1H, d).

### SYN-L-049, 1-(tert-butoxycarbonyl)-lyserqic acid diethylamide

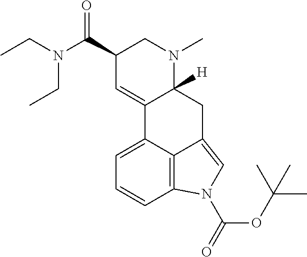 [SMILES: CCN(CC)C(=O)[C@@H]1C=C2c3cccc4c3c(cn4C(=O)OC(C)(C)C)C[C@H]2N(C)C1]

1H NMR: δ 1.22 (6H, t), 1.44 (9H, s), 2.57 (3H, s), 2.83-3.07 (4H, m), 3.22-3.40 (5H, m), 4.04 (1H, dd), 6.16 (1H, d), 6.97 (1H, dd), 7.11 (1H, dd), 7.61 (1H, ddd), 7.74 (1H, d).

### SYN-L-050, 1-(benzyloxycarbonyl)-lysergic acid diethylamide

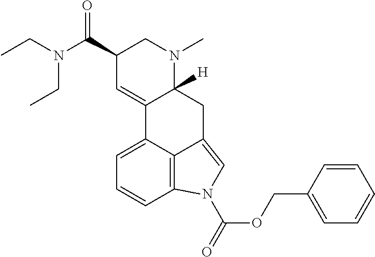 [SMILES: CCN(CC)C(=O)[C@@H]1C=C2c3cccc4c3c(cn4C(=O)OCc3ccccc3)C[C@H]2N(C)C1]

1H NMR: δ 1.22 (6H, t), 2.57 (3H, s), 2.83-3.07 (4H, m), 3.22-3.40 (5H, m), 4.04 (1H, dd), 5.09-5.19 (2H, m), 6.16 (1H, d), 6.97 (1H, dd), 7.11 (1H, dd), 7.29-7.45 (5H, m), 7.61 (1H, ddd), 7.74 (1H, d).

### SYN-L-051, 1-(bicyclo[1.1.1]pentylcarbonyl)-lyserqic acid diethylamide

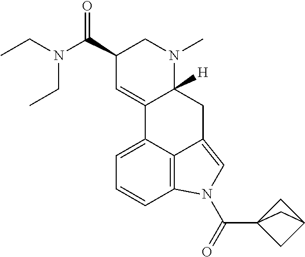 [SMILES: CCN(CC)C(=O)[C@@H]1C=C2c3cccc4c3c(cn4C(=O)C34CC(C3)C4)C[C@H]2N(C)C1]

1H NMR: δ-0.15-−0.02 (6H, m), 1.22 (6H, t), 2.19 (1H, m), 2.57 (3H, s), 2.81-3.06 (4H, m), 3.22-3.40 (5H, m), 4.00 (1H, dd), 6.16 (1H, d), 6.87-7.12 (2H, m), 7.52 (1H, ddd), 7.92 (1H, d).

### SYN-L-052, 1-(tetramethylcyclopropylmethanoyl)-lyserqic acid diethylamide

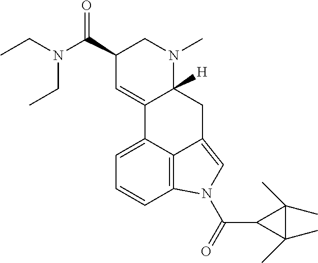 [SMILES: CCN(CC)C(=O)[C@@H]1C=C2c3cccc4c3c(cn4C(=O)C3C(C)(C)C3(C)C)C[C@H]2N(C)C1]

1H NMR: δ 1.12-1.28 (18H, m), 1.91 (1H, s), 2.57 (3H, s), 2.83-3.06 (4H, m), 3.22-3.40 (5H, m), 4.00 (1H, dd), 6.15 (1H, d), 6.88-7.14 (2H, m), 7.55 (1H, ddd), 7.97 (1H, d).

### SYN-L-053, 1-(2,3,3-trimethyl)butanoyl-lyserqic acid diethylamide

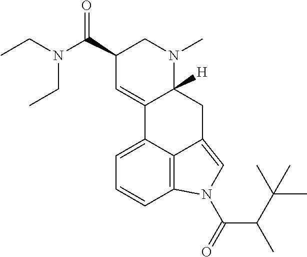 [SMILES: CCN(CC)C(=O)[C@@H]1C=C2c3cccc4c3c(cn4C(=O)C(C)C(C)(C)C)C[C@H]2N(C)C1]

1H NMR: δ 0.88 (9H, s), 1.07-1.28 (9H, m), 2.43-2.62 (4H, m), 2.83-3.06 (4H, m), 3.22-3.40 (5H, m), 4.00 (1H, dd), 6.16 (1H, d), 6.87-7.13 (2H, m), 7.53 (1H, ddd), 7.98 (1H, d).

### SYN-L-054, 1-(L-valinyl)-lyserqic acid diethylamide

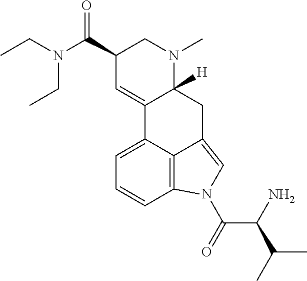 [SMILES: CCN(CC)C(=O)[C@@H]1C=C2c3cccc4c3c(cn4C(=O)[C@@H](N)C(C)C)C[C@H]2N(C)C1]

1H NMR: δ 0.78-0.90 (6H, m), 1.22 (6H, t), 1.95 (1H, m), 2.57 (3H, s), 2.81-3.06 (4H, m), 3.22-3.40 (5H, m), 3.84-4.06 (2H, m), 6.16 (1H, d), 6.88-7.13 (2H, m), 7.54 (1H, ddd), 8.01 (1H, d).

### SYN-L-055, 1-(cyclohexylacetyl)-lyserqic acid diethylamide

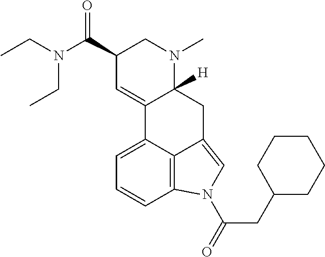 [SMILES: CCN(CC)C(=O)[C@@H]1C=C2c3cccc4c3c(cn4C(=O)CC3CCCCC3)C[C@H]2N(C)C1]

1H NMR: δ 1.16-1.65 (16H, m), 1.78 (1H, m), 2.52-2.72 (5H, m), 2.81-3.06 (4H, m), 3.22-3.40 (5H, m), 4.00 (1H, dd), 6.16 (1H, d), 6.87-7.12 (2H, m), 7.24 (1H, ddd), 7.93 (1H, d).

### SYN-L-056, 1-(4-tetrahydropyranacetyl)-lyserqic acid diethylamide

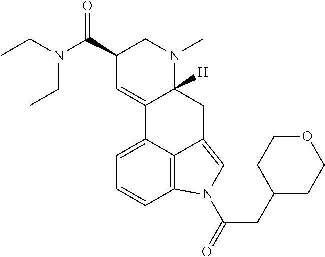 [SMILES: CCN(CC)C(=O)[C@@H]1C=C2c3cccc4c3c(cn4C(=O)CC3CCOCC3)C[C@H]2N(C)C1]

1H NMR: δ 1.22 (6H, t), 1.60-1.85 (4H, m), 2.36 (1H, m), 2.52-2.73 (5H, m), 2.81-3.06 (4H, m), 3.22-3.40 (5H, m), 3.44-3.63 (4H, m), 4.00 (1H, dd), 6.16 (1H, d), 6.87-7.12 (2H, m), 7.24 (1H, ddd), 7.93 (1H, d).

### SYN-L-057, 1-(quinuclidin-4-ylcarbonyl)-lyserqic acid diethylamide

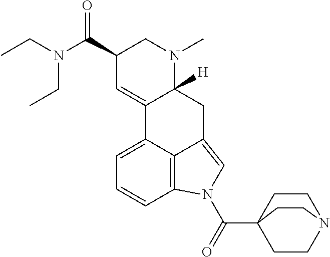 [SMILES: CCN(CC)C(=O)[C@@H]1C=C2c3cccc4c3c(cn4C(=O)C34CCN(CC3)CC4)C[C@H]2N(C)C1]

1H NMR: δ 1.22 (6H, t), 1.92-2.12 (6H, m), 2.57 (3H, s), 2.81-3.22 (10H, m), 3.22-3.40 (5H, m), 4.00 (1H, dd), 6.16 (1H, d), 6.88-7.13 (2H, m), 7.53 (1H, ddd), 8.00 (1H, d).

### SYN-L-058, 1-(N-methylacetamide)-lyserqic acid diethylamide

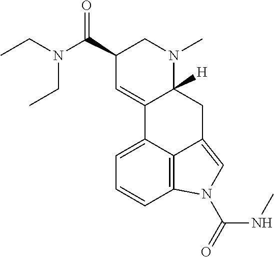 [SMILES: CCN(CC)C(=O)[C@@H]1C=C2c3cccc4c3c(cn4C(=O)NC)C[C@H]2N(C)C1]

1H NMR: δ 1.22 (6H, t), 2.57 (3H, s), 2.72 (3H, s), 2.82-3.07 (4H, m), 3.22-3.41 (5H, m), 3.99 (1H, dd), 6.15 (1H, d), 6.86-7.13 (2H, m), 7.54 (1H, ddd), 7.71 (1H, d).

### SYN-L-059, 1-placarbil-lyserqic acid diethylamide

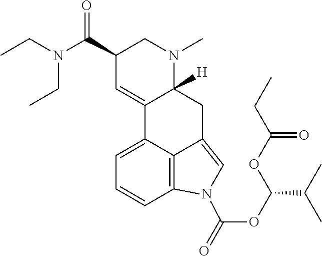 [SMILES: CCC(=O)O[C@@H](OC(=O)n1cc2c3c(cccc31)C1=C[C@@H](C(=O)N(CC)CC)CN(C)[C@@H]1C2)C(C)C]

1H NMR: δ 0.95-1.28 (18H, m), 2.09 (1H, m), 2.44 (1H, m), 2.57 (3H, s), 2.81-3.07 (4H, m), 3.22-3.41 (5H, m), 4.00 (1H, dd), 6.01 (1H, d), 6.16 (1H, d), 6.95 (1H, dd), 7.11 (1H, dd), 7.34 (1H, ddd), 7.74 (1H, d).

### SYN-L-060, 1-isopropoxyacetate-lyserqic acid diethylamide

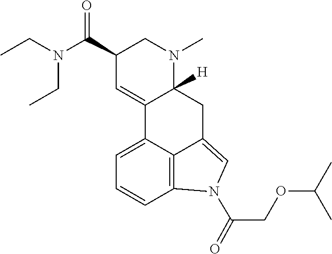 [SMILES: CCN(CC)C(=O)[C@@H]1C=C2c3cccc4c3c(cn4C(=O)COC(C)C)C[C@H]2N(C)C1]

1H NMR: δ 1.09-1.28 (12H, m), 2.57 (3H, s), 2.82-3.07 (4H, m), 3.22-3.43 (5H, m), 3.70 (1H, m), 4.02 (1H, dd), 4.51-4.61 (2H, m), 6.15 (1H, d), 6.86-7.12 (2H, m), 7.27 (1H, ddd), 8.02 (1H, d).

### SYN-L-061, 1-(3-ethoxypropanoyl)-lyserqic acid diethylamide

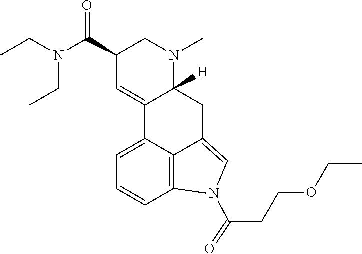 [SMILES: CCOCCC(=O)n1cc2c3c(cccc31)C1=C[C@@H](C(=O)N(CC)CC)CN(C)[C@@H]1C2]

1H NMR: δ 1.16-1.28 (9H, m), 2.57 (3H, s), 2.82-3.07 (6H, m), 3.22-3.47 (7H, m), 3.66-3.77 (2H, m), 4.01 (1H, dd), 6.15 (1H, d), 6.85-7.12 (2H, m), 7.23 (1H, ddd), 7.97 (1H, d).

### SYN-L-065, 1-propionyl-lyserqic acid ethylisopropylamide

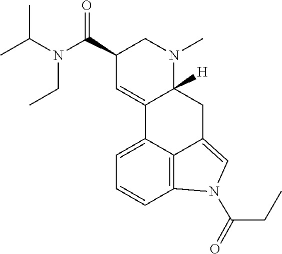 [SMILES: CCC(=O)n1cc2c3c(cccc31)C1=C[C@@H](C(=O)N(CC)C(C)C)CN(C)[C@@H]1C2]

1H NMR: δ 1.06 (3H, t), 1.16-1.28 (9H, m), 2.57 (3H, s), 2.62-2.73 (2H, m), 2.82-3.07 (4H, m), 3.18-3.30 (2H, m), 3.39 (1H, ddd), 4.02 (1H, dd), 4.34 (1H, m), 6.15 (1H, d), 6.85-7.12 (2H, m), 7.23 (1H, ddd), 7.92 (1H, d).

### SYN-L-066, 1-propionyl-lyserqic acid 2,4-dimethylazetidide

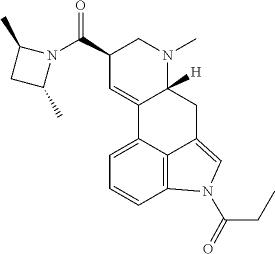 [SMILES: CCC(=O)n1cc2c3c(cccc31)C1=C[C@@H](C(=O)N3[C@H](C)C[C@H]3C)CN(C)[C@@H]1C2]

1H NMR: δ 1.00-1.16 (9H, m), 1.93 (2H, ddd), 2.57 (3H, s), 2.62-2.73 (2H, m), 2.82-3.08 (4H, m), 3.27 (1H, ddd), 3.83 (2H, m), 4.02 (1H, dd), 6.15 (1H, d), 6.85-7.12 (2H, m), 7.23 (1H, ddd), 7.92 (1H, d).

### SYN-L-067, 1-(2-furoyl)-lyserqic acid methylisopropylamide

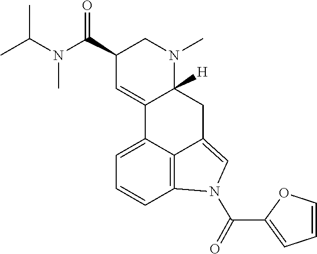 [SMILES: CC(C)N(C)C(=O)[C@@H]1C=C2c3cccc4c3c(cn4C(=O)c3ccco3)C[C@H]2N(C)C1]

1H NMR: δ 1.15-1.27 (6H, m), 2.57 (3H, s), 2.77 (3H, s), 2.82-3.07 (4H, m), 3.33 (1H, m), 4.04 (1H, dd), 4.33 (1H, m), 6.15 (1H, d), 6.65 (1H, dd), 6.88-7.13 (2H, m), 7.30 (1H, dd), 7.54 (1H, ddd), 7.84 (1H, dd), 8.00 (1H, d).

### SYN-L-068, 1-(2-furoyl)-lyserqic acid 2,4-dimethylazetidide

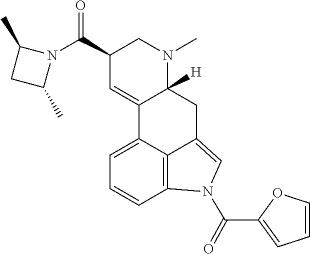 [SMILES: C[C@@H]1C[C@@H](C)N1C(=O)[C@@H]1C=C2c3cccc4c3c(cn4C(=O)c3ccco3)C[C@H]2N(C)C1]

1H NMR: δ 1.10 (6H, d), 1.93 (2H, m), 2.57 (3H, s), 2.82-3.08 (4H, m), 3.26 (1H, m), 3.81 (2H, m), 4.04 (1H, dd), 6.15 (1H, d), 6.65 (1H, dd), 6.88-7.13 (2H, m), 7.30 (1H, dd), 7.54 (1H, ddd), 7.84 (1H, dd), 8.00 (1H, d).

### SYN-L-073, 1-propionyl-6-allyl-6-nor-lyserqic acid methylisopropylamide

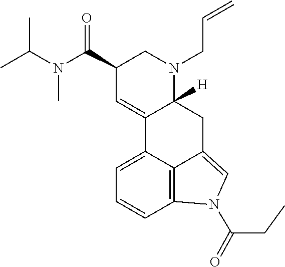 [SMILES: C=CCN1C[C@H](C(=O)N(C)C(C)C)C=C2c3cccc4c3c(cn4C(=O)CC)C[C@H]21]

1H NMR: δ 1.06 (3H, t), 1.15-1.27 (6H, d), 2.62-2.82 (5H, m), 2.82-3.11 (4H, m), 3.26-3.42 (3H, m), 3.97 (1H, dd), 4.33 (1H, m), 4.96-5.20 (2H, m), 5.71 (1H, m), 6.14 (1H, d), 6.86-7.12 (2H, m), 7.23 (1H, ddd), 7.96 (1H, d).

### SYN-L-074, 1-propionyl-6-allyl-6-nor-lyserqic acid 2,4-dimethylazetidide

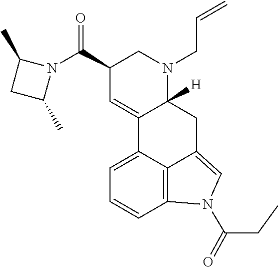 [SMILES: C=CCN1C[C@H](C(=O)N2[C@H](C)C[C@H]2C)C=C2c3cccc4c3c(cn4C(=O)CC)C[C@H]21]

1H NMR: δ 1.00-1.16 (9H, m), 1.93 (2H, m), 2.62-2.73 (2H, m), 2.82-3.11 (4H, m), 3.25-3.42 (3H, m), 3.76-4.04 (3H, m), 4.96-5.20 (2H, m), 5.71 (1H, ddt), 6.15 (1H, d), 6.86-7.12 (2H, m), 7.23 (1H, ddd), 7.96 (1H, d).

### SYN-L-077, 1-cyclopropanecarbonyl-2-bromo-lyserqic acid diethylamide

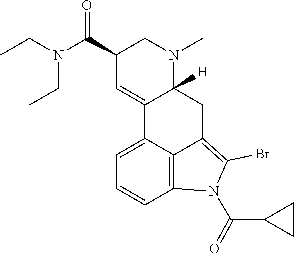 [SMILES: CCN(CC)C(=O)[C@@H]1C=C2c3cccc4c3c(c(Br)n4C(=O)C3CC3)C[C@H]2N(C)C1]

1H NMR: δ 0.95-1.11 (4H, m), 1.22 (6H, t), 2.38-2.62 (4H, m), 2.92-3.12 (4H, m), 3.22-3.46 (5H, m), 4.07 (1H, dd), 6.13 (1H, d), 6.90-7.11 (2H, m), 7.29 (1H, dd).

### SYN-L-078, 1-(2-furoyl)-2-bromo-lyserqic acid diethylamide

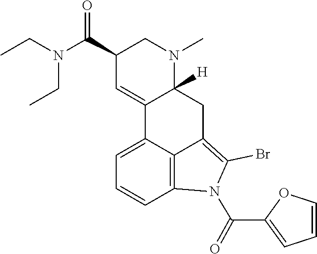 [SMILES: CCN(CC)C(=O)[C@@H]1C=C2c3cccc4c3c(c(Br)n4C(=O)c3ccco3)C[C@H]2N(C)C1]

1H NMR: δ 1.22 (6H, t), 2.57 (3H, s), 2.92-3.12 (4H, m), 3.22-3.45 (5H, m), 4.06 (1H, dd), 6.13 (1H, d), 6.65 (1H, dd), 6.96 (1H, dd), 7.10 (1H, dd), 7.22-7.36 (2H, m), 7.85 (1H, dd).

### SYN-L-079, 1-(3-phenylpropionyl)-2-bromo-lysergic acid diethylamide

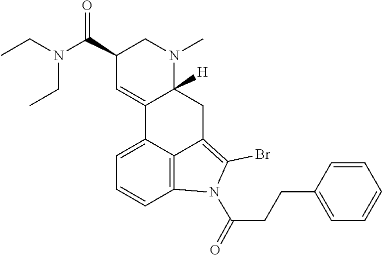 [SMILES: CCN(CC)C(=O)[C@@H]1C=C2c3cccc4c3c(c(Br)n4C(=O)CCc3ccccc3)C[C@H]2N(C)C1]

1H NMR: δ 1.22 (6H, t), 2.57 (3H, s), 2.69-2.81 (2H, m), 2.92-3.12 (6H, m), 3.22-3.46 (5H, m), 4.05 (1H, dd), 6.13 (1H, d), 6.89-7.35 (8H, m).

### SYN-L-080, 1-cyclopentanepropanoyl-2-bromo-lysergic acid diethylamide

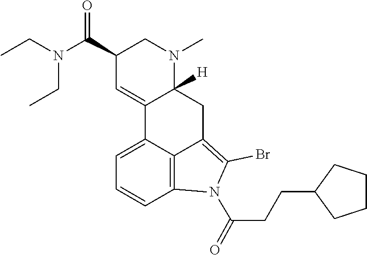 [SMILES: CCN(CC)C(=O)[C@@H]1C=C2c3cccc4c3c(c(Br)n4C(=O)CCC3CCCC3)C[C@H]2N(C)C1]

1H NMR: δ 1.22 (6H, t), 1.35-1.78 (10H, m), 1.98 (1H, m), 2.52-2.70 (5H, m), 2.92-3.12 (4H, m), 3.22-3.38 (5H, m), 4.03 (1H, dd), 6.13 (1H, d), 6.87-7.09 (2H, m), 7.28 (1H, dd).

### SYN-L-140, 1-propionyl-2-fluoro-lyserqic acid diethylamide

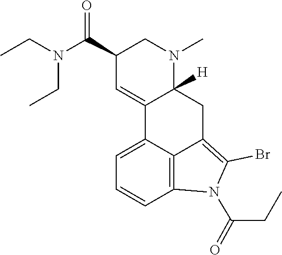 [SMILES: CCC(=O)n1c(Br)c2c3c(cccc31)C1=C[C@@H](C(=O)N(CC)CC)CN(C)[C@@H]1C2]

1H NMR: δ 1.05 (3H, t), 1.22 (6H, t), 2.52-2.71 (5H, m), 2.91-3.21 (4H, m), 3.22-3.39 (5H, m), 4.10 (1H, dd), 6.15 (1H, d), 6.97-7.23 (2H, m), 7.32 (1H, dd).

### SYN-L-142, 1-(2-furoyl)-6-ethyl-6-nor-lyserqic acid diethylamide

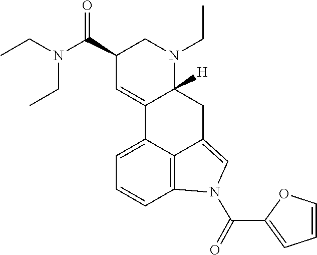 [SMILES: CCN(CC)C(=O)[C@@H]1C=C2c3cccc4c3c(cn4C(=O)c3ccco3)C[C@H]2N(CC)C1]

1H NMR: δ 1.01 (3H, t), 1.22 (6H, t), 2.59-2.70 (2H, q), 2.82-3.08 (4H, m), 3.22-3.39 (5H, m), 3.98 (1H, dd), 6.15 (1H, d), 6.65 (1H, dd), 6.88-7.13 (2H, m), 7.30 (1H, dd), 7.54 (1H, ddd), 7.84 (1H, dd), 8.00 (1H, d).

### SYN-L-143, 1-(2-furoyl)-6-allyl-6-nor-lyserqic acid diethylamide

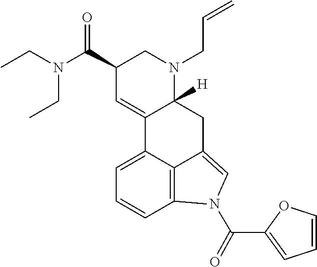 [SMILES: C=CCN1C[C@H](C(=O)N(CC)CC)C=C2c3cccc4c3c(cn4C(=O)c3ccco3)C[C@H]21]

1H NMR: δ 1.22 (6H, t), 2.84-3.11 (4H, m), 3.18-3.42 (7H, m), 4.02 (1H, dd), 4.96-5.20 (2H, m), 5.71 (1H, m), 6.13 (1H, d), 6.65 (1H, dd), 6.88-7.13 (2H, m), 7.30 (1H, dd), 7.54 (1H, ddd), 7.84 (1H, dd), 8.00 (1H, d).

### SYN-L-191, 1-(pent-5-yn-1-oyl)-lyserqic acid diethylamide

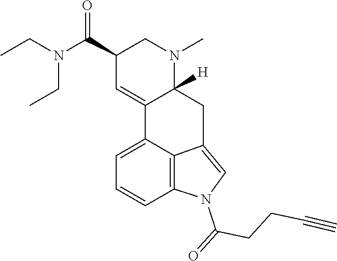 [SMILES: C#CCCC(=O)n1cc2c3c(cccc31)C1=C[C@@H](C(=O)N(CC)CC)CN(C)[C@@H]1C2]

1H NMR: δ 1.22 (6H, t), 2.52-2.67 (5H, m), 2.73-3.07 (7H, m), 3.22-3.43 (5H, m), 4.01 (1H, dd), 6.15 (1H, d), 6.85-7.12 (2H, m), 7.23 (1H, ddd), 7.97 (1H, d).

### SYN-L-192, 1-(cyclopent-3-enecarbonyl)-lyserqic acid diethylamide

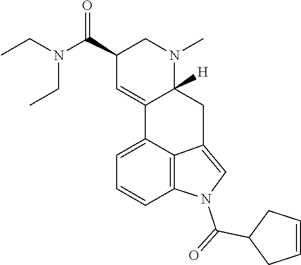 [SMILES: CCN(CC)C(=O)[C@@H]1C=C2c3cccc4c3c(cn4C(=O)C3CC=CC3)C[C@H]2N(C)C1]

1H NMR: δ 1.22 (6H, t), 2.44-2.63 (7H, m), 2.81-3.07 (4H, m), 3.22-3.43 (5H, m), 3.55 (1H, m), 4.02 (1H, dd), 5.98-6.20 (3H, m), 6.86-7.12 (2H, m), 7.24 (1H, ddd), 7.98 (1H, d).

### SYN-L-193, 1-(3-thiomethylpropionyl)-lyserqic acid diethylamide

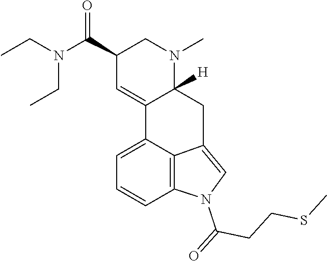 [SMILES: CCN(CC)C(=O)[C@@H]1C=C2c3cccc4c3c(cn4C(=O)CCSC)C[C@H]2N(C)C1]

1H NMR: δ 1.22 (6H, t), 2.20 (3H, s), 2.57 (3H, s), 2.66-3.06 (8H, m), 3.22-3.40 (5H, m), 4.00 (1H, dd), 6.15 (1H, d), 6.87-7.12 (2H, m), 7.24 (1H, ddd), 7.94 (1H, d).

### SYN-L-194, 1-(cyclopent-2-eneacetyl)-lyserqic acid diethylamide

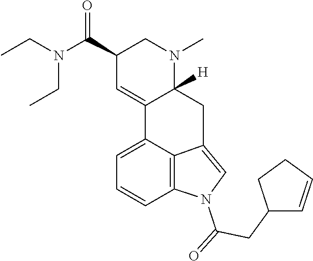 [SMILES: CCN(CC)C(=O)[C@@H]1C=C2c3cccc4c3c(cn4C(=O)CC3C=CCC3)C[C@H]2N(C)C1]

1H NMR: δ 1.22 (6H, t), 1.62 (1H, m), 1.86 (1H, m), 2.13-2.38 (2H, m), 2.57 (3H, s), 2.69-2.81 (2H, m), 2.82-3.08 (5H, m), 3.22-3.43 (5H, m), 4.02 (1H, dd), 5.87 (1H, ddd), 6.08-6.20 (2H, m), 6.86-7.12 (2H, m), 7.23 (1H, ddd), 7.97 (1H, d).

### SYN-L-195, 1-acetyl-lyserqic acid methylisopropylamide

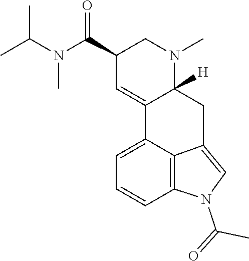 [SMILES: CC(=O)n1cc2c3c(cccc31)C1=C[C@@H](C(=O)N(C)C(C)C)CN(C)[C@@H]1C2]

1H NMR: δ 1.15-1.27 (6H, d), 2.35 (3H, s), 2.57 (3H, s), 2.72-3.07 (7H, m), 3.33 (1H, m), 4.01 (1H, dd), 4.33 (1H, m), 6.15 (1H, d), 6.85-7.11 (2H, m), 7.22 (1H, ddd), 7.91 (1H, d).

### SYN-L-196, 1-acetyl-lyserqic acid N-morpholinamide

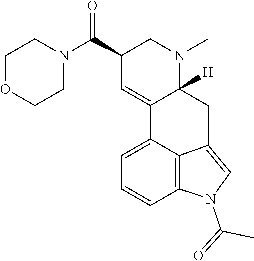 [SMILES: CC(=O)n1cc2c3c(cccc31)C1=C[C@@H](C(=O)N3CCOCC3)CN(C)[C@@H]1C2]

1H NMR: δ 2.35 (3H, s), 2.57 (3H, s), 2.82-3.06 (4H, m), 3.31 (1H, m), 3.54-3.71 (8H, m), 4.01 (1H, dd), 6.15 (1H, d), 6.85-7.11 (2H, m), 7.22 (1H, ddd), 7.91 (1H, d).

### SYN-L-197, 1-propionyl-lyserqic acid ethylcyclopropylamide

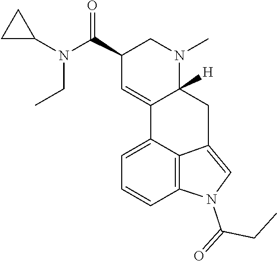 [SMILES: CCC(=O)n1cc2c3c(cccc31)C1=C[C@@H](C(=O)N(CC)C3CC3)CN(C)[C@@H]1C2]

1H NMR: δ 0.35-0.51 (2H, m), 0.55-0.71 (2H, m), 1.06 (3H, t), 1.25 (3H, t), 2.57 (3H, s), 2.62-2.73 (2H, m), 2.82-3.09 (5H, m), 3.29-3.43 (3H, m), 4.02 (1H, dd), 6.15 (1H, d), 6.85-7.12 (2H, m), 7.23 (1H, ddd), 7.92 (1H, d).

### SYN-L-198, 1-propionyl-lyserqic acid-(S,S)-trans-2,5-dimethylpyrrolidide

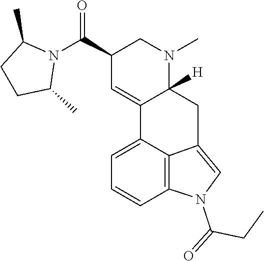 [SMILES: CCC(=O)n1cc2c3c(cccc31)C1=C[C@@H](C(=O)N3[C@H](C)CC[C@H]3C)CN(C)[C@@H]1C2]

1H NMR: δ 1.00-1.18 (9H, m), 1.75-2.01 (4H, m), 2.57 (3H, s), 2.62-2.73 (2H, m), 2.82-3.08 (4H, m), 3.31 (1H, ddd), 3.76 (2H, m), 4.02 (1H, dd), 6.14 (1H, d), 6.85-7.12 (2H, m), 7.23 (1H, ddd), 7.92 (1H, d).

### SYN-L-200, 1-cyclopropanecarbonyl-lyserqic acid-(S,S)-trans-2,5-dimethylpyrrolidide

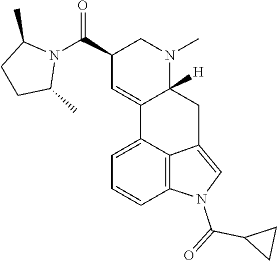 [SMILES: C[C@@H]1CC[C@@H](C)N1C(=O)[C@@H]1C=C2c3cccc4c3c(cn4C(=O)C3CC3)C[C@H]2N(C)C1]

1H NMR: δ 0.87-1.04 (4H, m), 1.12 (6H, d), 1.76-1.99 (4H, m), 2.32 (1H, t), 2.57 (3H, s), 2.81-3.08 (4H, m), 3.24 (1H, m), 3.80 (2H, m), 3.96 (1H, dd), 6.13 (1H, d), 6.88-7.13 (2H, m), 7.54 (1H, ddd), 7.92 (1H, d).

### SYN-L-201, 1-propionyl-6-(2-fluoroethyl)-6-nor-lyserqic acid diethylamide

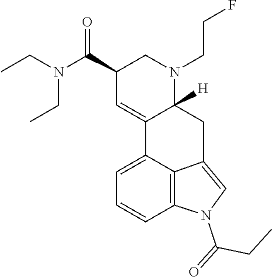 [SMILES: CCC(=O)n1cc2c3c(cccc31)C1=C[C@@H](C(=O)N(CC)CC)CN(CCF)[C@@H]1C2]

1H NMR: δ 1.06 (3H, t), 1.22 (6H, t), 2.62-2.73 (2H, q), 2.82-3.11 (6H, m), 3.22-3.38 (5H, m), 4.02 (1H, dd), 4.34-4.45 (2H, m), 6.15 (1H, d), 6.86-7.12 (2H, m), 7.23 (1H, ddd), 7.93 (1H, d).

### SYN-L-203, 1-propionyl-6-methallyl-6-nor-lyserqic acid diethylamide

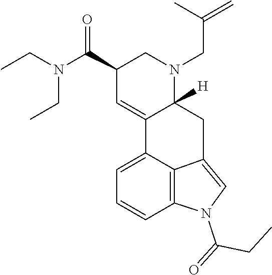 [SMILES: C=C(C)CN1C[C@H](C(=O)N(CC)CC)C=C2c3cccc4c3c(cn4C(=O)CC)C[C@H]21]

1H NMR: δ 1.06 (3H, t), 1.22 (6H, t), 1.65 (3H, s), 2.62-2.73 (2H, q), 2.83-3.12 (4H, m), 3.18-3.38 (7H, m), 3.97 (1H, dd), 4.97-5.07 (2H, d), 6.14 (1H, d), 6.86-7.12 (2H, m), 7.23 (1H, ddd), 7.96 (1H, d).

### SYN-L-204, 1-propionyl-6-cyclopropylmethyl-6-nor-lyserqic acid diethylamide

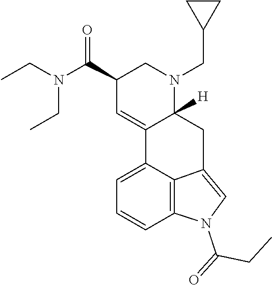 [SMILES: CCC(=O)n1cc2c3c(cccc31)C1=C[C@@H](C(=O)N(CC)CC)CN(CC3CC3)[C@@H]1C2]

1H NMR: δ 0.22-0.38 (4H, m), 0.87 (1H, m), 1.06 (3H, t), 1.22 (6H, t), 2.36-2.48 (2H, d), 2.62-2.73 (2H, q), 2.82-3.11 (4H, m), 3.18-3.37 (5H, m), 3.99 (1H, dd), 6.14 (1H, d), 6.86-7.12 (2H, m), 7.23 (1H, ddd), 7.96 (1H, d).

### SYN-L-205, 1-(2-thiophenecarbonyl)-6-ethyl-6-nor-lyserqic acid methylisopropylamide

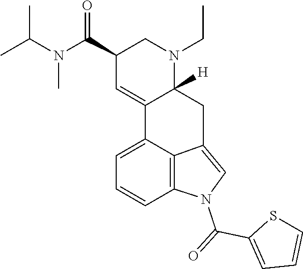 [SMILES: CCN1C[C@H](C(=O)N(C)C(C)C)C=C2c3cccc4c3c(cn4C(=O)c3cccs3)C[C@H]21]

1H NMR: δ 1.01 (3H, t), 1.15-1.27 (6H, d), 2.59-2.70 (2H, q), 2.77 (3H, s), 2.85-3.07 (4H, m), 3.28 (1H, ddd), 3.86 (1H, dd), 4.33 (1H, m), 6.15 (1H, d), 6.87-7.12 (2H, m), 7.22 (1H, dd), 7.52 (1H, ddd), 7.79 (1H, dd), 7.92-8.04 (2H, m).

### SYN-L-206, 1-propionyl-2-methyl-lyserqic acid diethylamide

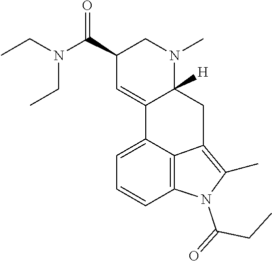 [SMILES: CCC(=O)n1c(C)c2c3c(cccc31)C1=C[C@@H](C(=O)N(CC)CC)CN(C)[C@@H]1C2]

1H NMR: δ 1.05 (3H, t), 1.22 (6H, t), 2.47 (3H, s), 2.57 (3H, s), 2.62-2.73 (2H, q), 2.82-3.09 (4H, m), 3.22-3.40 (5H, m), 4.04 (1H, dd), 6.15 (1H, d), 6.86-7.22 (3H, m).

### SYN-L-208, 1-propionyl-2-cyclopropyl-lyserqic acid diethylamide

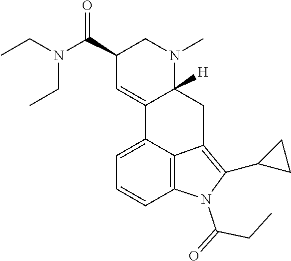 [SMILES: CCC(=O)n1c(C2CC2)c2c3c(cccc31)C1=C[C@@H](C(=O)N(CC)CC)CN(C)[C@@H]1C2]

1H NMR: δ 0.97-1.28 (13H, m), 2.57 (3H, s), 2.63-2.74 (2H, q), 2.79-3.09 (5H, m), 3.22-3.41 (5H, m), 4.05 (1H, dd), 6.15 (1H, d), 6.93 (1H, dd), 7.04-7.26 (2H, m).

### SYN-L-209, 1-propionyl-2-trifluoromethyl-lyserqic acid diethylamide

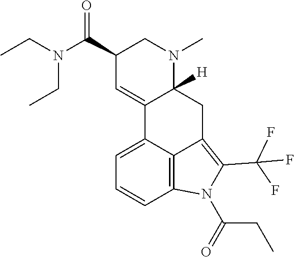 [SMILES: CCC(=O)n1c(C(F)(F)F)c2c3c(cccc31)C1=C[C@@H](C(=O)N(CC)CC)CN(C)[C@@H]1C2]

1H NMR: δ 1.06 (3H, t), 1.22 (6H, t), 2.57 (3H, s), 2.63-2.74 (2H, q), 2.85-3.07 (4H, m), 3.22-3.43 (5H, m), 4.02 (1H, dd), 6.15 (1H, d), 6.95 (1H, dd), 7.09-7.37 (2H, m).

### SYN-L-210, 1-cyclopropanecarbonyl-9,10-cyclopropyl-lyserqic acid diethylamide

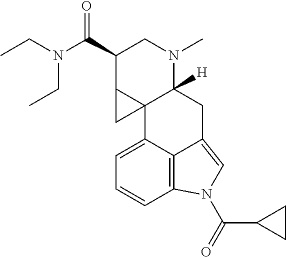 [SMILES: CCN(CC)C(=O)[C@H]1CN(C)[C@@H]2Cc3cn(C(=O)C4CC4)c4cccc(c34)C23CC13]

1H NMR: δ 0.86-1.08 (6H, m), 1.22 (6H, t), 1.44 (1H, m), 2.15 (1H, m), 2.57 (3H, s), 2.73-2.97 (5H, m), 3.22-3.40 (5H, m), 6.90-7.09 (2H, m), 7.47 (1H, ddd), 7.88 (1H, d).

### SYN-L-211, 1-propionyl-9,10-cyclopropyl-lyserqic acid diethylamide

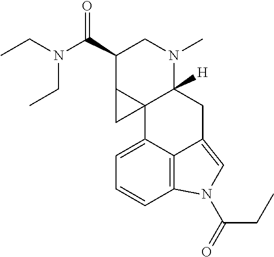 [SMILES: CCC(=O)n1cc2c3c(cccc31)C13CC1[C@@H](C(=O)N(CC)CC)CN(C)[C@@H]3C2]

1H NMR: δ 0.91-1.11 (5H, m), 1.22 (6H, t), 1.44 (1H, m), 2.57 (3H, s), 2.60 (2H, q), 2.71-3.00 (5H, m), 3.22-3.42 (5H, m), 6.90-7.08 (2H, m), 7.45 (1H, ddd), 7.75 (1H, d).

### SYN-L-212, 1-propionyl-9,10-dihydro-lyserqic acid diethylamide

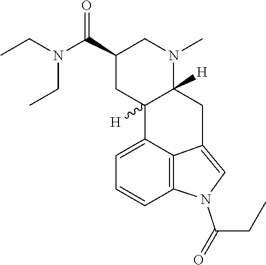 [SMILES: CCC(=O)n1cc2c3c(cccc31)C1C[C@@H](C(=O)N(CC)CC)CN(C)[C@@H]1C2]

1H NMR: δ 1.05 (3H, t), 1.22 (6H, t), 2.10 (1H, m), 2.39 (1H, m), 2.58 (3H, s), 2.60 (2H, q), 2.69-2.91 (4H, m), 2.96-3.16 (2H, m), 3.17-3.29 (4H, q), 3.36 (1H, m), 6.87-7.04 (3H, m), 7.73 (1H, d).

### SYN-L-213, 1-propionyl-6-ethyl-6-nor-lyserqic acid 2,4-dimethylazetidide

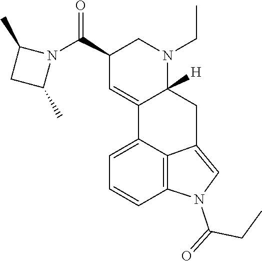 [SMILES: CCC(=O)n1cc2c3c(cccc31)C1=C[C@@H](C(=O)N3[C@H](C)C[C@H]3C)CN(CC)[C@@H]1C2]

1H NMR: δ 0.96-1.16 (12H, m), 1.93 (2H, m), 2.59-2.73 (4H, m), 2.81-3.08 (4H, m), 3.30 (1H, m), 3.82 (2H, m), 3.96 (1H, dd), 6.16 (1H, d), 6.86-7.12 (2H, m), 7.23 (1H, ddd), 7.92 (1H, d).

### SYN-L-214, 1-propionyl-6-ethyl-6-nor-lyserqic acid 2-butyl amide

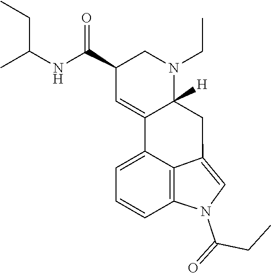 [SMILES: CCC(=O)n1cc2c3c(cccc31)C1=C[C@@H](C(=O)NC(C)CC)CN(CC)[C@@H]1C2]

1H NMR: δ 0.84 (3H, t), 0.95-1.11 (6H, t), 1.38 (3H, d), 1.53-1.66 (2H, m), 2.58-2.73 (4H, m), 2.81-3.07 (4H, m), 3.34 (1H, m), 3.96 (1H, dd), 4.18 (1H, m), 6.15 (1H, d), 6.85-7.12 (2H, m), 7.23 (1H, ddd), 7.93 (1H, d).

### SYN-L-215, 1-propionyl-6-ethyl-6-nor-lyserqic acid ethyl-(2-trifluoroethyl)-amide

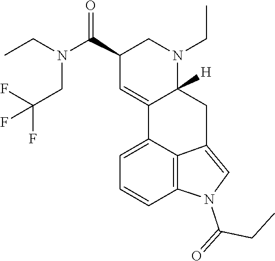 [SMILES: CCC(=O)n1cc2c3c(cccc31)C1=C[C@@H](C(=O)N(CC)CC(F)(F)F)CN(CC)[C@@H]1C2]

1H NMR: δ 0.95-1.11 (6H, t), 1.22 (3H, t), 2.58-2.73 (4H, m), 2.81-3.08 (4H, m), 3.20-3.37 (3H, m), 3.68-3.78 (2H, s), 4.00 (1H, dd), 6.15 (1H, d), 6.87-7.12 (2H, m), 7.23 (1H, ddd), 7.90 (1H, d).

### SYN-L-216, 1-(3,4-methylenedioxybenzoyl)-lyserqic acid diethylamide

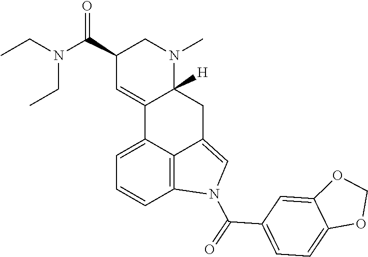 [SMILES: CCN(CC)C(=O)[C@@H]1C=C2c3cccc4c3c(cn4C(=O)c3ccc4c(c3)OCO4)C[C@H]2N(C)C1]

1H NMR: δ 1.22 (6H, t), 2.57 (3H, s), 2.82-3.07 (4H, m), 3.22-3.41 (5H, m), 4.00 (1H, dd), 6.09-6.22 (3H, d), 6.85-7.11 (3H, m), 7.25 (1H, ddd), 7.57-7.78 (2H, m), 7.91 (1H, d).

### SYN-L-217, 1-(2-benzothiophenecarbonyl)-lyserqic acid diethylamide

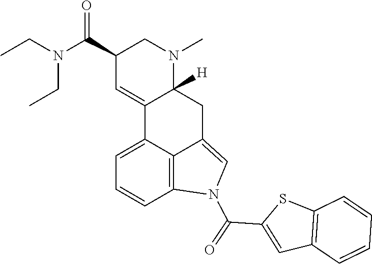 [SMILES: CCN(CC)C(=O)[C@@H]1C=C2c3cccc4c3c(cn4C(=O)c3cc4ccccc4s3)C[C@H]2N(C)C1]

1H NMR: δ 1.22 (6H, t), 2.57 (3H, s), 2.82-3.07 (4H, m), 3.22-3.41 (5H, m), 4.00 (1H, dd), 6.15 (1H, d), 6.85-7.12 (2H, m), 7.26 (1H, ddd), 7.35-7.51 (2H, m), 7.89-8.10 (4H, m).

### SYN-L-218, 1-spirobicyclobutanoyl-lyserqic acid diethylamide

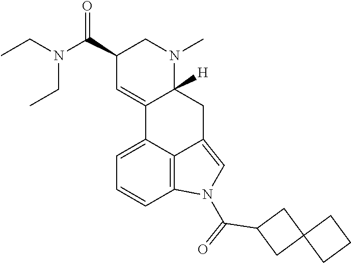 [SMILES: CCN(CC)C(=O)[C@@H]1C=C2c3cccc4c3c(cn4C(=O)C3CC4(CCC4)C3)C[C@H]2N(C)C1]

1H NMR: δ 1.16-1.48 (10H, m), 1.62-1.86 (4H, m), 2.14-2.35 (2H, m), 2.57 (3H, s), 2.81-3.07 (4H, m), 3.16-3.43 (6H, m), 4.02 (1H, dd), 6.15 (1H, d), 6.92 (1H, dd), 7.05 (1H, dd), 7.24 (1H, ddd), 7.97 (1H, d).

### SYN-L-219, 1-trifluoroacetyl-lyserqic acid diethylamide

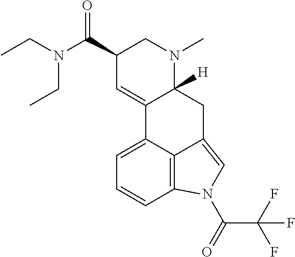 [SMILES: CCN(CC)C(=O)[C@@H]1C=C2c3cccc4c3c(cn4C(=O)C(F)(F)F)C[C@H]2N(C)C1]

1H NMR: δ 1.22 (6H, t), 2.57 (3H, s), 2.82-3.07 (4H, m), 3.22-3.41 (5H, m), 4.02 (1H, dd), 6.15 (1H, d), 6.93 (1H, dd), 7.07 (1H, dd), 7.58 (1H, ddd), 8.12 (1H, d).

### SYN-L-220, 1-(N-pyrrolidine)acetyl-lyserqic acid diethylamide

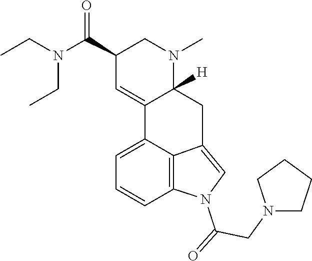 [SMILES: CCN(CC)C(=O)[C@@H]1C=C2c3cccc4c3c(cn4C(=O)CN3CCCC3)C[C@H]2N(C)C1]

1H NMR: δ 1.22 (6H, t), 1.80-2.00 (4H, m), 2.57 (3H, s), 2.82-3.17 (8H, m), 3.22-3.43 (5H, m), 3.81 (2H, s), 4.02 (1H, dd), 6.15 (1H, d), 6.92 (1H, dd), 7.05 (1H, dd), 7.25 (1H, ddd), 8.00 (1H, d).

### SYN-L-221, 1-thienothiophenecarbonyl-lyserqic acid diethylamide

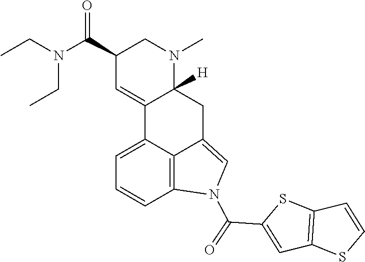 [SMILES: CCN(CC)C(=O)[C@@H]1C=C2c3cccc4c3c(cn4C(=O)c3cc4sccc4s3)C[C@H]2N(C)C1]

1H NMR: δ 1.22 (6H, t), 2.57 (3H, s), 2.82-3.07 (4H, m), 3.22-3.41 (5H, m), 4.00 (1H, dd), 6.15 (1H, d), 6.92 (1H, dd), 7.04 (1H, dd), 7.26 (1H, ddd), 7.56 (1H, dd), 7.72 (1H, dd), 7.96 (1H, d), 8.08 (1H, t).

### SYN-L-222, 1-(3-(trimethylsilyl)prop-2-ynoyl-lyserqic acid diethylamide

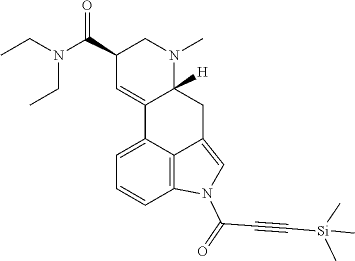 [SMILES: CCN(CC)C(=O)[C@@H]1C=C2c3cccc4c3c(cn4C(=O)C#C[Si](C)(C)C)C[C@H]2N(C)C1]

1H NMR: δ 0.11 (9H, s), 1.22 (6H, t), 2.57 (3H, s), 2.81-3.07 (4H, m), 3.22-3.41 (5H, m), 4.01 (1H, dd), 6.16 (1H, d), 6.93 (1H, dd), 7.06 (1H, dd), 7.56 (1H, ddd), 7.91 (1H, d).

### SYN-L-223, 1-(3-tetrahydropyrancarbonyl-lyserqic acid diethylamide

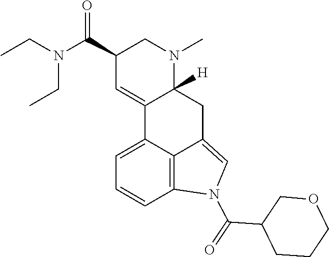 [SMILES: CCN(CC)C(=O)[C@@H]1C=C2c3cccc4c3c(cn4C(=O)C3CCCOC3)C[C@H]2N(C)C1]

1H NMR: δ 1.22 (6H, t), 1.55-1.83 (3H, m), 2.07 (1H, m), 2.57 (3H, s), 2.82-3.07 (4H, m), 3.22-3.64 (8H, m), 3.77 (1H, dd), 3.90-4.08 (2H, m), 6.15 (1H, d), 6.86-7.12 (2H, m), 7.24 (1H, ddd), 7.99 (1H, d).

### SYN-L-224, 1-(3-butenoyl)-lyserqic acid diethylamide

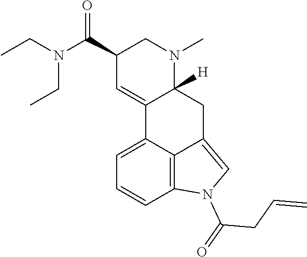 [SMILES: C=CCC(=O)n1cc2c3c(cccc31)C1=C[C@@H](C(=O)N(CC)CC)CN(C)[C@@H]1C2]

1H NMR: δ 1.22 (6H, t), 2.57 (3H, s), 2.81-3.07 (4H, m), 3.22-3.46 (7H, m), 4.02 (1H, dd), 4.97-5.21 (2H, m), 6.03-6.22 (2H, m), 6.86-7.12 (2H, m), 7.24 (1H, ddd), 7.97 (1H, d).

### SYN-L-225, 1-(tetrahydro-2-thiophenecarbonyl)-lyserqic acid diethylamide

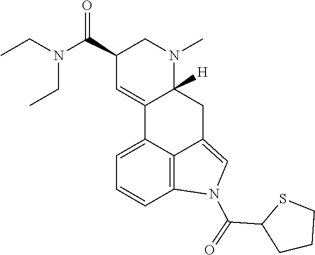 [SMILES: CCN(CC)C(=O)[C@@H]1C=C2c3cccc4c3c(cn4C(=O)C3CCCS3)C[C@H]2N(C)C1]

1H NMR: δ 1.22 (6H, t), 1.80 (1H, m), 1.89-2.10 (3H, m), 2.57 (3H, s), 2.82-3.41 (11H, m), 4.02 (1H, dd), 4.56 (1H, dd), 6.15 (1H, d), 6.86-7.13 (2H, m), 7.26 (1H, ddd), 8.01 (1H, d).

### SYN-L-226, 1-(tetrahydro-3-furoyl)-lyserqic acid diethylamide

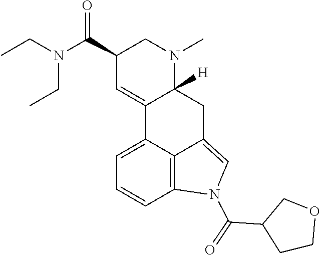 [SMILES: CCN(CC)C(=O)[C@@H]1C=C2c3cccc4c3c(cn4C(=O)C3CCOC3)C[C@H]2N(C)C1]

1H NMR: δ 1.22 (6H, t), 1.98 (1H, m), 2.25 (1H, m), 2.57 (3H, s), 2.82-3.07 (4H, m), 3.22-3.41 (5H, m), 3.59-4.12 (6H, m), 6.15 (1H, d), 6.86-7.12 (2H, m), 7.24 (1H, ddd), 7.99 (1H, d).

### SYN-L-227, 1-(methylthio)acetyl-lyserqic acid diethylamide

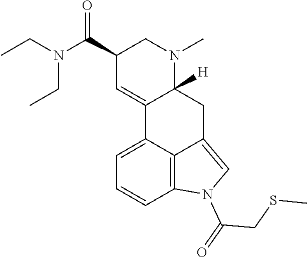 [SMILES: CCN(CC)C(=O)[C@@H]1C=C2c3cccc4c3c(cn4C(=O)CSC)C[C@H]2N(C)C1]

1H NMR: δ 1.22 (6H, t), 2.15 (3H, s), 2.57 (3H, s), 2.82-3.07 (4H, m), 3.22-3.41 (5H, m), 3.95-4.08 (3H, m), 6.15 (1H, d), 6.86-7.12 (2H, m), 7.25 (1H, ddd), 8.00 (1H, d).

### SYN-L-228, 1-(5-bromo-2-thiophenecarbonyl)-lyserqic acid diethylamide

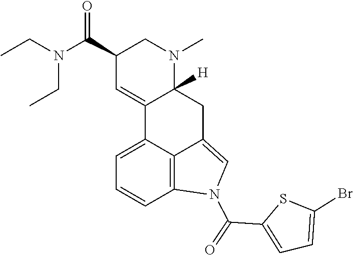 [SMILES: CCN(CC)C(=O)[C@@H]1C=C2c3cccc4c3c(cn4C(=O)c3ccc(Br)s3)C[C@H]2N(C)C1]

1H NMR: δ 1.22 (6H, t), 2.57 (3H, s), 2.82-3.07 (4H, m), 3.22-3.41 (5H, m), 4.00 (1H, dd), 6.15 (1H, d), 6.86-7.12 (2H, m), 7.40 (1H, d), 7.53 (1H, ddd), 7.83-8.02 (2H, m).

### SYN-L-229, 1-(1,2-dimethylcyclobutane-1-carbonyl-lyserqic acid diethylamide

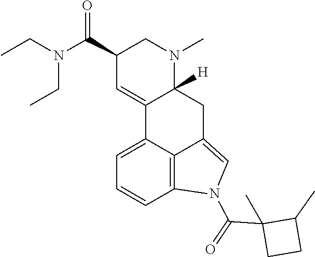 [SMILES: CCN(CC)C(=O)[C@@H]1C=C2c3cccc4c3c(cn4C(=O)C3(C)CCC3C)C[C@H]2N(C)C1]

1H NMR: δ 0.96 (3H, d), 1.06-1.28 (9H, m), 1.62-2.00 (4H, m), 2.15 (1H, m), 2.57 (3H, s), 2.82-3.07 (4H, m), 3.22-3.41 (5H, m), 4.02 (1H, dd), 6.15 (1H, d), 6.92 (1H, dd), 7.05 (1H, dd), 7.25 (1H, ddd), 7.99 (1H, d).

### SYN-L-230, 1-(1,4-dimethylspiro[2.3]hexane-1-carbonyl)-lyserqic acid diethylamide

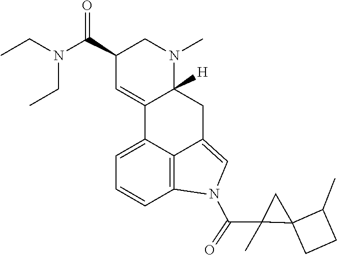 [SMILES: CCN(CC)C(=O)[C@@H]1C=C2c3cccc4c3c(cn4C(=O)C3(C)CC34CCC4C)C[C@H]2N(C)C1]

1H NMR: δ 0.90-1.14 (5H, m), 1.22 (6H, t), 1.34 (3H, s), 1.46-1.76 (4H, m), 2.00 (1H, m), 2.57 (3H, s), 2.82-3.07 (4H, m), 3.22-3.41 (5H, m), 4.02 (1H, dd), 6.15 (1H, d), 6.86-7.13 (2H, m), 7.55 (1H, ddd), 7.98 (1H, d).

### SYN-L-231, 1-(1,2-dimethylbicyclo[1.1.0]butane-2-carbonyl)-lyserqic acid diethylamide

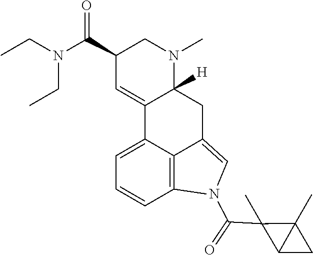 [SMILES: CCN(CC)C(=O)[C@@H]1C=C2c3cccc4c3c(cn4C(=O)C3(C)C4CC43C)C[C@H]2N(C)C1]

1H NMR: δ 0.56 (1H, dd), 0.62-0.81 (2H, m), 1.16-1.33 (9H, m), 1.42 (3H, s), 2.57 (3H, s), 2.82-3.07 (4H, m), 3.22-3.41 (5H, m), 4.02 (1H, dd), 6.15 (1H, d), 6.93 (1H, dd), 7.13 (1H, dd), 7.56 (1H, ddd), 8.00 (1H, d).

### SYN-L-232, 1-(4-oxopentanoyl)-lyserqic acid diethylamide

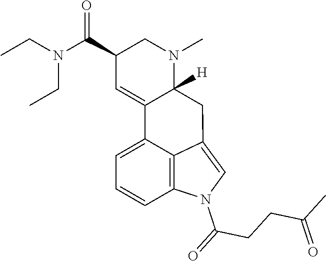 [SMILES: CCN(CC)C(=O)[C@@H]1C=C2c3cccc4c3c(cn4C(=O)CCC(C)=O)C[C@H]2N(C)C1]

¹H NMR: δ 1.22 (6H, t), 2.15 (3H, s), 2.57 (3H, s), 2.68-2.80 (2H, m), 2.82-3.07 (6H, m), 3.22-3.43 (5H, m), 4.01 (1H, dd), 6.15 (1H, d, J=5.3 Hz), 6.92 (1H, dd), 7.05 (1H, dd), 7.23 (1H, ddd), 7.97 (1H, d).

### SYN-L-233, 1-(3-selenophenecarbonyl)-lyserqic acid diethylamide

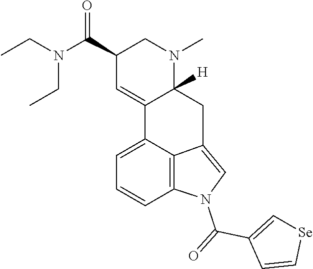 [SMILES: CCN(CC)C(=O)[C@@H]1C=C2c3cccc4c3c(cn4C(=O)c3cc[se]c3)C[C@H]2N(C)C1]

¹H NMR: δ 1.22 (6H, t), 2.57 (3H, s), 2.81-3.07 (4H, m), 3.22-3.41 (5H, m), 4.01 (1H, dd), 6.15 (1H, d), 6.86-7.13 (2H, m), 7.53 (1H, dd), 7.54 (1H, ddd), 7.94 (1H, dd), 7.96 (1H, dd), 7.98 (1H, d).

### SYN-L-234, 1-(ferrocenecarbonyl)-lyserqic acid diethylamide

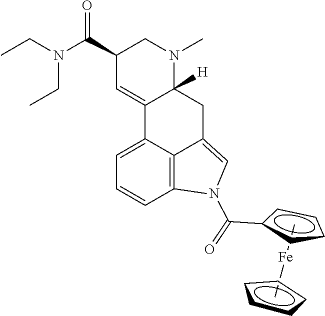

¹H NMR: δ 1.22 (6H, t), 2.44 (3H, s) 2.57 (3H, s), 2.81-3.07 (4H, m), 3.22-3.41 (5H, m), 4.01 (1H, dd), 4.23 (5H, s), 4.53 (2H, m), 4.82 (2H, m), 6.15 (1H, d), 6.91 (1H, dd), 7.02 (1H, ddd), 7.04 (1H, dd), 7.85 (1H, d).

### SYN-L-235, 1-(ferrocenecarbonyl)-6-allyl-6-nor-lysercic acid diethylamide

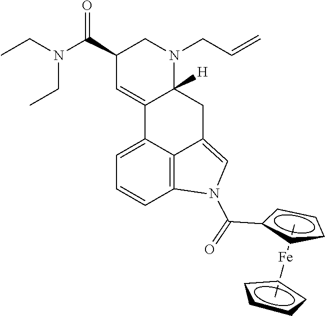

¹H NMR: δ 1.22 (6H, t), 2.44 (3H, s) 2.82-3.11 (4H, m), 3.18-3.42 (7H, m), 3.96 (1H, dd), 4.96-5.20 (2H, m), 5.71 (1H, ddt), 4.23 (5H, s), 4.53 (2H, m), 4.82 (2H, m), 6.15 (1H, d), 6.91 (1, dd), 7.03 (1H, ddd), 7.04 (1H, dd), 7.85 (1, d).

### Compounds of the Invention Act as Prodrugs

Male C57BL/6J mice (6-8 weeks old) obtained from Jackson Laboratories (Bar Harbor, ME, USA) were housed in a vivarium at the University of California San Diego (UCSD), an AAALAC-approved animal facility that meets all Federal and State requirements for care and treatment of laboratory animals. Mice were housed up to four per cage in a climate-controlled room on a reverse-light cycle (lights on at 1900 h, off at 0700 h) and were provided with ad libitum access to food and water, except during behavioral testing. Testing was conducted between 1000 and 1800 h. All animal experiments were carried out in accordance with NIH guidelines and were approved by the UCSD animal care committee.

To prepare the drug solutions, a stock solution of each lysergamide was prepared (1 mg lysergamide was dissolved in 100 μl DMSO) and stored at −20° C. On the day of each experiment, the stock solution was diluted into sterile saline at a concentration of 0.2 mg/ml. Solutions used for administration to mice were prepared fresh daily.

Blood samples were taken from mice injected with 1 mg/kg of the test compound IP, with a 30 min pretreatment time. The vehicle was sterile saline, 5 ml/kg injection volume. All of the drugs were dissolved in a DMSO stock solution (0.1 ml added per mg) and then diluted into saline. Blood was collected in tubes coated with K2EDTA. Within 30 min of collection, the blood was centrifuged (2,000 rpm) for 12 min at 4° C., and then plasma was collected in 50-μl aliquots, flash frozen with dry ice, and stored at −80° C. Samples were tested with LCMS to determine concentration of LSD released at the 30 min time point. Values are average of 3 samples taken from different mice.

Sample preparation by solid-phase extraction: Sample preparation was performed as described previously by Halberstadt et al. (Halberstadt A L, Chatha M, Klein A K, McCorvy J D, Meyer M R, Wagmann L, Stratford A, Brandt S D (2020) Pharmacological and biotransformation studies of 1-acyl-substituted derivatives of d-lysergic acid diethylamide (LSD). Neuropharmacology 172: 107856. DOI: 10.1016/j.neuropharm.2019.107856), with minor modifications. 10 μl of methanolic LSD-d3 (as internal standard, final plasma concentration 5 ng/ml) were added to 0.1 ml of mouse plasma, diluted with 2.9 ml of purified water, mixed for 15 s on a rotary shaker, and loaded on a HCX cartridge (130 mg, 3 ml) previously conditioned with 1 ml of methanol and 1 ml of purified water. After extraction, the cartridge was washed with 1 ml of purified water, 1 ml of 0.01 M aqueous hydrochloric acid, and 2 ml of methanol. Reduced pressure was applied until the cartridge was dry and the analytes were eluted with 1 ml of a freshly prepared mixture of methanol-aqueous ammonia (98:2, v/v) into a reaction tube. The eluate was evaporated to dryness under a stream of nitrogen at 70° C. and the residue was dissolved in 25 μl of a mixture of 10 mM aqueous ammonium formate-acetonitrile (1:1, v/v) containing 0.1% formic acid. The LSD plasma concentration was determined using an LC-ion trap MS apparatus and an LC-high-resolution MS/MS apparatus by calculating the mean value of both analyses.

LC-ion trap MS apparatus for LSD quantification: Samples were analyzed using a ThermoFisher Scientific (TF, Dreieich, Germany) LXQ linear ion trap MS, coupled to a TF Accela ultra high performance LC (UHPLC) system consisting of a degasser, a quaternary pump, and an autosampler. Gradient elution was performed on a TF Hypersil GOLD C18 column (100 mm×2.1 mm inner diameter, 1.9 μm particle size). The mobile phase consisted of 10 mM aqueous ammonium formate plus 0.1% formic acid (pH 3.4, eluent A) and acetonitrile plus 0.1% formic acid (eluent B). The flow rate was set to 0.5 ml/min and the following gradient was used: 0-2.0 min 2% B, 2.0-4.0 min to 80% B, 4.0-6.0 min hold 80% B, 6.0-6.5 min to 90% B, 6.5-7.0 min hold 90% B, 7.0-10.0 min hold 80% B, 10.0-17.0 hold 2% B. Analyses were performed in a targeted acquisition mode with an inclusion list, where MS2 spectra of given precursor ions (LSD and LSD-d3) were recorded. The injection volume was 10 μl each. The MS was equipped with a heated electrospray ionization II (HESI-II) source, other conditions were as follows: positive ionization mode; sheath gas, nitrogen at flow rate of 34 arbitrary units (AU); auxiliary gas, nitrogen at flow rate of 11 AU; vaporizer temperature, 250° C.; source voltage, 3.00 kV; ion transfer capillary temperature, 300° C.; capillary voltage, 38 V; tube lens voltage, 110 V; automatic gain control (AGC) target, 5,000 ions for MS2; data type, centroid; normalized collision energy, 35.0; wideband activation, enabled; isolation width, m/z 1.5. TF Xcalibur Qual Browser software version 2.0.7 was used for data evaluation and LSD concentration was determined comparing the peak areas of LSD and LSD-d3 within the same run.

LC-high resolution MS/MS apparatus for LSD quantification: Analyses were performed using the procedure of Wagmann et al. (H H, Meyer M R (2019) In vitro metabolic fate of nine LSD-based new psychoactive substances and their analytical detectability in different urinary screening procedures. Anal Bioanal Chem 411: 4751-4763. DOI: 10.1007/s00216-018-1558-9), with minor modifications. A TF Dionex UltiMate 3000 Rapid Separation (RS) UHPLC system with a quaternary UltiMate 3000 RS pump and an UltiMate 3000 RS autosampler was used, controlled by the TF Chromeleon software version 6.80, and coupled to a TF Q-Exactive Plus equipped with a HESI-II source. Mass calibration was performed prior to analysis according to the manufacturer's recommendations using external mass calibration.

Gradient elution was performed on a TF Accucore PhenylHexyl column (100 mm×2.1 mm inner diameter, 2.6 μm particle size). The mobile phases consisted of 2 mM aqueous ammonium formate containing formic acid (0.1%, v/v) and acetonitrile (1%, v/v, pH 3, eluent A) and 2 mM ammonium formate in acetonitrile/methanol (50:50, v/v) containing formic acid (0.1%, v/v) and water (1%, v/v, eluent B). The gradient and flow rate were programmed as follows: 0-10 min 10% B to 50% B, 10-12 min hold 98% B, and 12-14 min hold 10% B, constantly at a flow rate of 0.5 mL/min. HESI-II source conditions were as follows: heater temperature, 438° C.; ion transfer capillary temperature, 269° C.; sheath gas, 53 AU; auxiliary gas, 14 AU; sweep gas, 3 AU; spray voltage, 3.50 kV, and S-lens RF level, 60.0. Mass spectrometric analysis was performed in positive full-scan mode and targeted MS2 mode using an inclusion list containing the accurate masses of protonated LSD and LSD-d3. The injection volume was 5 μL each. The settings for full-scan data acquisition were as follows: resolution, 35,000; AGC target, 1e6; maximum injection time (IT), 120 ms; scan range, m/z 100-700. The settings for the targeted MS2 were as follows: resolution, 17,500; AGC target, 2e5; maximum IT, 250 ms; isolation window, m/z 1.0; high-collision dissociation cell with stepped normalized collision energy 17.5, 35.0, 52.5. TF Xcalibur Qual Browser software version 2.2 SP1.48 was used for data evaluation and the LSD concentration was determined comparing the peak areas of LSD and LSD-d3 within the same run.

All lysergamides tested were hydrolyzed to LSD after IP injection in mice. Each lysergamide was administered at an IP dose of 1 mg/kg and plasma samples were collected 30 min later. LSD was quantified using LSD-d3 as an internal standard. Plasma levels of LSD ranged from 14.5 f 3.0 for SYN-L-006, up to 186.7 f 5.5 ng/ml (mean±SEM) for SYN-L-027 as shown in Table 1.

TABLE 1

| | | | Normalized |
|---|---|---|---|
| | | | LSD plasma |
| | Dose | LSD plasma | concentration |
| | injected IP | concentration | (ng/ml LSD per |
| Drug code | (μmol/kg) | (mean ± SEM) | μmol/kg injected) |
| SYN-L-003 | 2.05 | 67.3 ± 15.6 | 32.8 ± 7.6 |
| SYN-L-004 | 1.64 | 37.0 ± 3.8 | 22.6 ± 2.3 |
| SYN-L-005 | 1.99 | 100.3 ± 5.5 | 50.4 ± 2.8 |
| SYN-L-006 | 1.80 | 14.5 ± 3.0 | 8.1 ± 1.7 |
| SYN-L-010 | 1.82 | 133.3 ± 14.5 | 73.3 ± 8.0 |
| SYN-L-012 | 1.65 | 80.7 ± 21.7 | 48.9 ± 13.2 |
| SYN-L-013 | 1.76 | 25.0 ± 4.7 | 14.2 ± 2.7 |
| SYN-L-021 | 1.50 | 150.0 ± 25.2 | 100.0 ± 16.8 |
| SYN-L-027 | 1.66 | 186.7 ± 14.5 | 112.0 ± 8.8 |
| SYN-L-028 | 1.63 | 92.7 ± 14.6 | 56.9 ± 9.0 |
| SYN-L-036 | 1.40 | 176.7 ± 12.0 | 126.0 ± 8.6 |

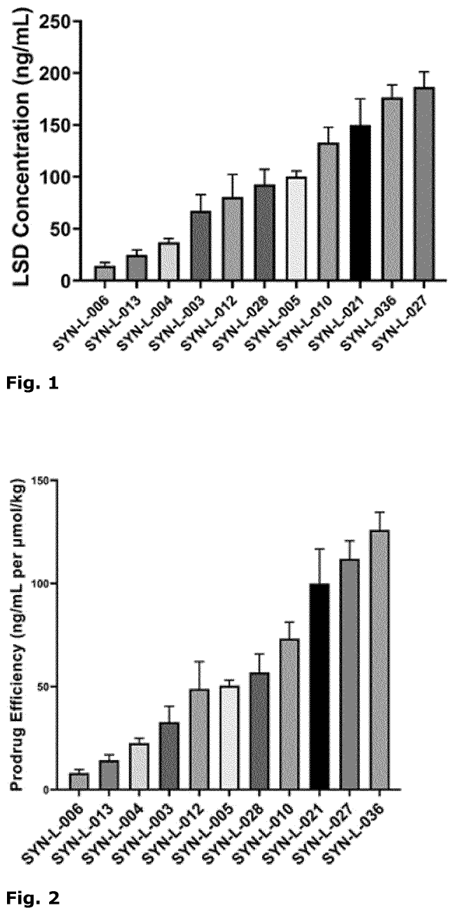

The results are further depicted in FIG. 1. The efficiency of conversion to LSD across the 12 drugs was calculated by normalizing the results based on the molar mass of each drug that was injected. FIG. 2 shows the data after normalization; the conversion efficacy of each compound is shown as ng/ml LSD in plasma per μmol/kg injected. SYN-L-036 showed the highest conversion efficiency, with 126.0±8.6 ng/ml LSD generated per μmol/kg of drug injected.

### Compounds of the Invention Produce a Head-Twitch Response in Mice

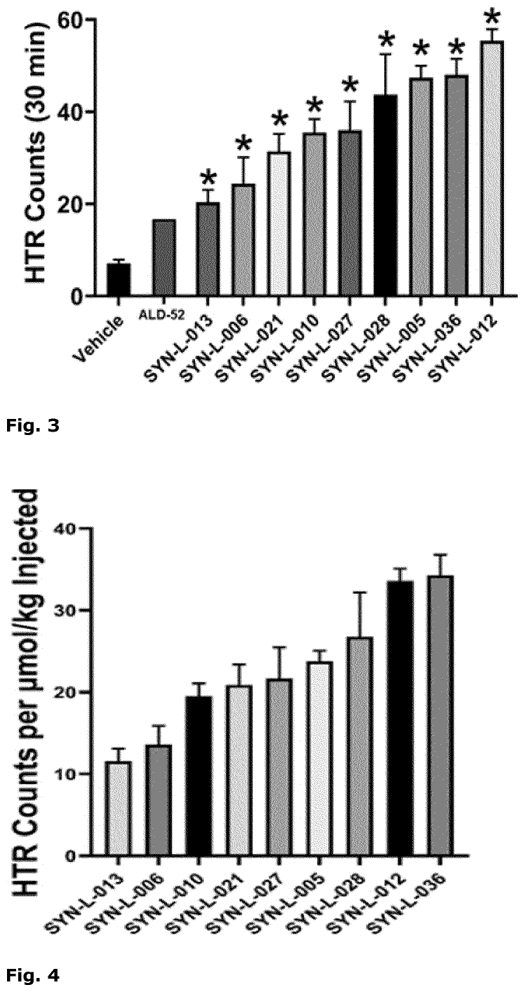

A head-twitch response (HTR) study was conducted in male C57BL/6J mice to assess whether the lysergamides can induce 5-HT2A receptor activation in vivo. Each lysergamide was tested at an IP dose of 1 mg/kg. The results of are summarized in FIG. 3. There was a significant main effect of drug in the experiment (F(12,62)=33.00, p<0.0001). Post-hoc pairwise comparisons confirmed that all nine of the lysergamides tested in the experiments induced a significant increase of HTR counts compared to vehicle control. A correlation analysis was conducted to evaluate the relationship between drug effects on the HTR and LSD plasma concentrations. The magnitude of the HTR induced by each drug was calculated by subtracting the baseline level of HTR (measured using the vehicle group) from the drug responses.

The HTR was assessed using a head-mounted magnet and a magnetometer detection coil. The mice were anesthetized with ketamine and xylazine, a small incision was made in the scalp, and a small neodymium magnet was attached to the dorsal surface of the cranium using dental cement. Following a recovery period of several weeks, HTR experiments were carried out in a well-lit room. Ten groups of male C₅₇BL/6J mice were injected IP with vehicle (n=18) or 1 mg/kg of each lysergamide (n=5 mice/group, 45 total). The injection volume was 5 ml/kg. The mice were immediately placed in a HTR recording chamber and activity was recorded continuously for 30 min. Coil voltage was low-pass filtered (1 kHz cutoff frequency), amplified, digitized (20 kHz sampling rate, 16-bit ADC resolution), and saved to disk using a Powerlab/8SP data acquisition system with LabChart software ver. 7.3.2 (ADInstruments, Colorado Springs, CO, USA).

Head twitches were identified using a validated technique based on artificial intelligence (Halberstadt A L (2020) Automated detection of the head-twitch response using wavelet scalograms and a deep convolutional neural network. Sci Rep 10: 8344). To detect the behavior, events in the recordings are transformed into a visual representation in the time-frequency domain (a scalogram), deep features are extracted using the pretrained convolutional neural network (CNN) ResNet-50, and then the images are classified using a Support Vector Machine (SVM) algorithm. Head-twitch counts were analyzed using one-way analyses of variance (ANOVA). Post hoc comparisons were made using Dunnett's test. Significance was demonstrated by surpassing an o level of 0.05. The results are summarized in FIGS. 3 and 4.

### Compounds of the Invention Show Attenuated Activity Compared to ALD-52 in Humans

Compounds of the invention were tested on volunteers who were experienced with the use of the related compound ALD-52. For each compound, a solution was prepared by dissolving the tartrate salt in deionized water to produce a concentration either of 1 mg/ml of the tartrate salt, or calculated to be equivalent to 1 mg/ml of the free base. This was either administered directly, or was prepared into standardised dosage units by pipetting 0.1 ml of solution onto a square of perforated blotter paper, which was placed overnight in a desiccator and allowed to evaporate to dryness. The results are shown in Table 2. Certain compounds of the invention exhibit attenuated psychedelic effects compared to ALD-52, and may be useful for psychedelic-assisted psychotherapy but with reduced hallucinogenic side effects compared to commonly used agents such as LSD or psilocybin. Certain other compounds of the invention produce no psychedelic effects at any dose tested, but exhibit useful anti-headache properties.

TABLE 2

| | | Dosage tested | | Maximum | |
|---|---|---|---|---|---|
| | | (administered as | | psychedelic | |
| | | tartrate salt, | | effect | |
| | Substitution | calculated as free- | | produced | |
| Compound | for ALD-52 | base equivalent) | | (ALD-52 = 1) | Duration |
| SYN-L-003 | Partial | 162 | μg | 0.25 | Similar to ALD- |
| | substitution | | | | 52 |
| SYN-L-004 | Partial | 83-166 | μg | 0.25 | Slightly longer |
| | substitution | | | | duration than |
| | | | | | ALD-52 |
| SYN-L-008 | Partial | 106-510 | μg | 0.25 | Similar to ALD- |
| | substitution | | | | 52 |
| SYN-L-024 | Partial | 370 | μg | 0.25 | Shorter duration |
| | substitution | | | | than ALD-52 |
| SYN-L-042 | Partial | 300 | μg | 0.25 | Similar to ALD- |
| | substitution | | | | 52 |
| SYN-L-043 | Partial | 300 | μg | 0.25 | Similar to ALD- |
| | substitution | | | | 52 |
| SYN-L-073 | Partial | 100-400 | μg | 0.5 | Similar to ALD- |
| | substitution | | | | 52 |
| SYN-L-203 | Partial | 500 | μg | 0.25 | Similar to ALD- |
| | substitution | | | | 52 |
| SYN-L-196 | Partial | 300-500 | μg | 0.5 | Similar to ALD- |
| | substitution | | | | 52 |
| SYN-L-197 | Partial | 300 | μg | 0.5 | Similar to ALD- |
| | substitution | | | | 52 |
| SYN-L-014 | No substitution | 170 | μg | 0 | Similar to ALD- |
| | as psychedelic, | | | | 52 |
| | but alleviates | | | | |
| | migraine | | | | |
| | headache | | | | |
| SYN-L-017 | No substitution | 75-5160 | μg | 0 | Similar to ALD- |

| | |
|---|---|
| as psychedelic, | 52 |
| but alleviates | |
| cluster | |
| headache | |

### Compounds of the Invention Alleviate Cluster Headache Symptoms in Humans

Eighteen volunteers were recruited through cluster headache support groups in the Netherlands. Age range of volunteers was between 23 to 67 (average 45.17), 14 males and 4 females. All volunteers had previous experience using psychedelic drugs such as LSD or psilocybin to alleviate cluster headache attacks, as well as prescribed medications including verapamil, prednisone, sumatriptan, topiramate and indomethacin. Volunteers were asked to discontinue use of prescribed medications for five days preceding the commencement of the study and for the eight-week duration.

Participants were supplied standardised 3.0 mg dosage units of SYN-L-017 as the tartrate salt (equivalent to 2.58 mg of SYN-L-017 freebase), measured into capsules for oral administration. These were tested as a preventative for chronic cluster headache attacks. One 3 mg capsule was administered intermittently in a pattern of one day on, two days off for a period of eight weeks (using the protocol described in Karst et al. (Karst M, et al. The non-hallucinogen 2-bromo-lysergic acid diethylamide as preventative treatment for cluster headache: An open, non-randomized case series. Cephalalgia 2010; 30(9):1140-1144. DOI: 10.1177/0333102410363490)) and the volunteers were asked to keep a diary to track the frequency and self-rated severity of cluster headache attacks on a scale of 1-10.

At the start of the study, baseline severity of cluster headache attacks was rated at an average of 8.78/10 with an average of 3.44 attacks per day. After four weeks, severity was rated at an average of 4.46/10 with an average of 2.13 attacks per day. At the end of the eight weeks this had dropped to an average severity of 3.72/10 with an average of 1.78 attacks per day. Overall, 91.67% of volunteers reported improvement of cluster headache symptoms when taking SYN-L-017, with attacks perceived as being less frequent, of shorter duration and of reduced intensity compared to baseline. 75% of volunteers reported no side effects of any significance while 25% reported side effects including dizziness, nausea, fatigue, difficulty concentrating and insomnia. In all cases these were perceived as being mild and no volunteers reported side effects rated as moderate or severe. No side effects consistent with psychedelic effects were reported. 83.33% of volunteers thought SYN-L-017 was more effective than the prescribed medication they normally used to alleviate cluster headache attacks.

1-15. (canceled)

16. A compound of formula (1) 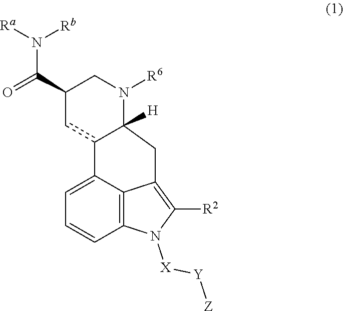
wherein
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C₁₋₁₂ alkylthio, aryl, heterocyclyl, heteroaryl, metallocenyl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, alkylthio, cycloalkyl, cycloalkoxy, aryl, heterocyclyl, heteroaryl or metallocenyl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂;
R² is selected from hydrogen, C₁₋₃ alkyl, C₃₋₅ cycloalkyl, oxetane, C₁₋₃ haloalkyl, C₁₋₃ alkylthio, halogen, cyano, COOH and CONH₂;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or R^{a} and R^{b}, together with the adjacent nitrogen atom, form a 3 to 7 membered heterocyclic ring, wherein said heterocyclic ring may be optionally substituted with C₁₋₃ alkyl or halogen; and [symbol]
is a single or double bond or a cyclopropyl ring;
provided that compounds of formula (1) having a linear C₁₋₅ acyl group, a valeroyl group or a cyclopropylcarbonyl group, as —XYZ are excluded; or a pharmaceutically acceptable salt thereof.

17. The compound of claim 16, wherein R² is H, R⁶ is methyl, R^{a}═R^{b}=ethyl, [symbol] is a double bond, X is C═O, Y is a bond or a C₁₋₄ alkylene residue, which may be substituted with C₁₋₃ alkyl or halogen, and Z is selected from hydrogen, C₁₋₁₂ alkyl, C₂₋₁₂ alkenyl, C₂₋₁₂ alkynyl, C₃₋₇ cycloalkyl, C₄₋₇ cycloalkenyl, C₁₋₁₂ alkoxy, aryl, heterocyclyl, heteroaryl, COOH, and CO—C₁₋₆ alkyl.

18. The compound of claim 17, wherein Y is a chemical bond, and Z is furan-2-yl, furan-3-yl, tetrahydrofuran-2-yl, pyridin-3-yl, pyridin-4-yl, thiophen-2-yl, oxetan-3-yl or oxan-4-yl.

19. The compound of claim 17, wherein Y is CH₂CH₂, and Z is COOCH₃, phenyl, cyclohexyl or cyclopentyl.

20. The compound of claim 17, wherein Y is OCH(CH₃), and Z is O—CO—CH(CH₃)₂.

21. The compound of claim 17, wherein Y is CH₂CH₂, and Z is hydrogen, phenyl or cyclopentyl.

22. The compound of claim 16, wherein R² is H, R⁶ is methyl, R^{a}═R^{b}=ethyl, [symbol] is a double bond, X is C═O, Y is a bond or a C₁₋₄ alkylene residue, which may be substituted with C₁₋₃ alkyl or halogen, and Z is C₅₋₂₀ alkyl.

23. The compound of claim 16, wherein R² is Br.

24. The compound of claim 16, which is selected from the group consisting of: 1-ethylcarbamoyl-lysergic acid diethylamide (SYN-L-003), 1-dodecanoyl-lysergic acid diethylamide (SYN-L-004), 1-(2-furoyl)-lysergic acid diethylamide (SYN-L-005), 1-(3-phenylpropionoyl)-lysergic acid diethylamide (SYJN-L-006), 1-(o-toluoyl)-lysergic acid diethylamide (SYN-L-008), 1-nicotinoyl-lysergic acid diethylamide (SYN-L-010), 1-methylsuccinyl-lysergic acid diethylamide (SYN-L-012), 1-enacarbil-lysergic acid diethylamide (SYN-L-013), 1-cyclopropanesulfonyl-lysergic acid diethylamide (SYN-L-014), 1-methoxyacetyl-lysergic acid diethylamide (SYN-L-015), 1-propionyl-2-bromo-lysergic acid diethylamide (SYN-L-017), 1-benzoyl-lysergic acid diethylamide (SYN-L-018), 1-(2-thiophenecarbonyl)-lysergic acid diethylamide (SYN-L-021), 1-phenylacetyl-lysergic acid diethylamide (SYN-L-022), 1-(1-adamantanecarbonyl)-lysergic acid diethylamide (SYN-L-023), 1-pivaloyl-lysergic acid diethylamide (SYN-L-024), 1-isovaleryl-lysergic acid diethylamide (SYN-L-026), 1-hexanoyl-lysergic acid diethylamide (SYN-L-027), 1-cyclopentanepropanoyl-lysergic acid diethylamide (SYN-L-028), 1-cyclohexanecarbonyl-lysergic acid diethylamide (SYN-L-029), 1-cyclopentanecarbonyl-lysergic acid diethylamide (SYN-L-030), 1-cyclobutanecarbonyl-lysergic acid diethylamide (SYN-L-031), 1-isobutyryl-lysergic acid diethylamide (SYN-L-032), 1-(5-methyl-2-furoyl)-lysergic acid diethylamide (SYN-L-034), 1-(5-tert-butyl-2-furoyl)-lysergic acid diethylamide (SYN-L-035), 1-(tetrahydro-2-furoyl)-lysergic acid diethylamide (SYN-L-036), 1-(3-furoyl)-lysergic acid diethylamide (SYN-L-037), 1-(3-thiophenecarbonyl)-lysergic acid diethylamide (SYN-L-038), 1-isonicotinoyl-lysergic acid diethylamide (SYN-L-039), 1-(3-oxetanoyl)-lysergic acid diethylamide (SYN-L-040), 1-(4-tetrahydropyranoyl)-lysergic acid diethylamide (SYN-L-041), 1-(4-morpholinylbutyryl)-lysergic acid diethylamide (SYN-L-046), 1-(4-morpholinecarbonyl)-lysergic acid diethylamide (SYN-L-047), 1-(p-tosyl)-lysergic acid diethylamide (SYN-L-048), 1-(tert-butoxycarbonyl)-lysergic acid diethylamide (SYN-L-049), 1-(benzyloxycarbonyl)-lysergic acid diethylamide (SYN-L-050), 1-(bicyclo[1.1.1]pentylcarbonyl)-lysergic acid diethylamide (SYN-L-051), 1-(tetramethylcyclopropylmethanoyl)-lysergic acid diethylamide (SYN-L-052), 1-(2,3,3-trimethyl)butanoyl-lysergic acid diethylamide (SYN-L-053), 1-(4-tetrahydropyranacetyl)-lysergic acid diethylamide (SYN-L-056), 1-isopropoxyacetate-lysergic acid diethylamide (SYN-L-060), 1-(3-ethoxypropanoyl)-lysergic acid diethylamide (SYN-L-061), 1-(3-phenylpropionyl)-2-bromo-lysergic acid diethylamide (SYN-L-079), 1-(pent-5-yn-1-oyl)-lysergic acid diethylamide (SYN-L-191), 1-(cyclopent-3-enecarbonyl)-lysergic acid diethylamide (SYN-L-192), 1-(3-thiomethylpropionyl)-lysergic acid diethylamide (SYN-L-193), 1-(cyclopent-2-eneacetyl)-lysergic acid diethylamide (SYN-L-194), 1-(2-benzothiophenecarbonyl)-lysergic acid diethylamide (SYN-L-217), 1-spirobicyclobutanoyl-lysergic acid diethylamide (SYN-L-218), 1-trifluoroacetyl-lysergic acid diethylamide (SYN-L-219), 1-(N-pyrrolidine)acetyl-lysergic acid diethylamide (SYN-L-220), 1-thienothiophenecarbonyl-lysergic acid diethylamide (SYN-L-221), 1-(3-tetrahydropyrancarbonyl)-lysergic acid diethylamide (SYN-L-223), 1-(3-butenoyl)-lysergic acid diethylamide (SYN-L-224), 1-(tetrahydro-2-thiophenecarbonyl)-lysergic acid diethylamide (SYN-L-225), 1-(tetrahydro-3-furoyl)-lysergic acid diethylamide (SYN-L-226), 1-(methylthio)acetyl-lysergic acid diethylamide (SYN-L-227), 1-(5-bromo-2-thiophenecarbonyl)-lysergic acid diethylamide (SYN-L-228), 1-(1,2-dimethylcyclobutane-1-carbonyl)-lysergic acid diethylamide (SYN-L-229), 1-(1,4-dimethylspiro[2.3]hexane-1-carbonyl)-lysergic acid diethylamide (SYN-L-230) and 1-(1,2-dimethylbicyclo[1.1.0]butane-2-carbonyl)-lysergic acid diethylamide (SYN-L-231), 1-(4-oxopentanoyl)-lysergic acid diethylamide (SYN-L-232), 1-(3-selenophenecarbonyl)-lysergic acid diethylamide (SYN-L-233), 1-(ferrocenecarbonyl)-lysergic acid diethylamide (SYN-L-234) and 1-(ferrocenecarbonyl)-6-allyl-6-nor-lysergic acid diethylamide (SYN-L-235), or a pharmaceutically acceptable salt thereof.

25. The compound of claim 24, which is selected from the group selected of SYN-L-005, SYN-L-012, SYN-L-013 and SYN-036, or a pharmaceutically acceptable salt thereof.

26. A pharmaceutical composition or medicament comprising a compound of claim 16, or a pharmaceutically acceptable salt thereof and a pharmaceutically acceptable carrier.

27. The pharmaceutical composition or medicament of claim 26, wherein the compound of formula (1) has modulated or reduced hallucinogenic side effects as compared to LSD and 1-acetyl-LSD.

28. The composition or medicament of claim 27, which is for the treatment of diseases in a subject requiring a modulated or reduced hallucinogenic side effects as compared to LSD and 1-acetyl-LSD selected from the group of diseases consisting of:
(i) migraine headache;
(ii) cluster headache;
(iii) depression;
(iv) anxiety;
(v) post-traumatic stress disorder;
(vi) glaucoma in a subject; and
(vii) rheumatoid arthritis.

29. A method for treating diseases requiring a modulated or reduced hallucinogenic side effects as compared to LSD and 1-acetyl-LSD, said method comprising administering a compound of claim 16, or a pharmaceutically acceptable salt of said compound, or a pharmaceutical composition comprising said compound or the pharmaceutically acceptable salt of said compound and a pharmaceutically acceptable carrier, to a subject in need of such treatment.

30. The method of claim 29 which is for the treatment of diseases in a subject selected from the group of diseases consisting of:
(i) migraine headache;
(ii) cluster headache;
(iii depression;
(iv) anxiety;
(v) post-traumatic stress disorder;
(vi) glaucoma; and
(vii) rheumatoid arthritis.

31. A method for producing a compound of formula (1) 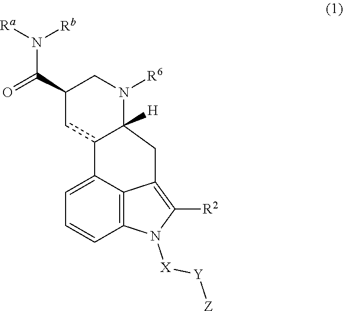
wherein
X is selected from CO and SO₂;
Y is selected from a single chemical bond, O, S, and a C₁₋₈ alkylene, C₂₋₈ alkenylene, C₂₋₄ alkynylene or O—C₁₋₆ alkylene residue, wherein each of said alkylenes, alkenylenes or alkynylenes may be optionally substituted with C₁₋₃ alkyl or halogen;
Z is selected from hydrogen, halogen, cyano, C₁₋₃ trialkylsilyl, C₁₋₂₀ alkyl, C₂₋₂₀ alkenyl, C₂₋₂₀ alkynyl, C₃₋₇ cycloalkyl, C₄₋₇ cycloalkenyl, C₁₋₁₂ alkoxy, C₁₋₁₂ alkylthio, aryl, heterocyclyl, heteroaryl, metallocenoyl, COOH, CO—C₁₋₆ alkyl, CO—C₃₋₇ cycloalkyl, O—CO—C₁₋₆ alkyl, O—CO—C₃₋₇ cycloalkyl, CO—C₁₋₆ alkoxy or CO—C₃₋₇ cycloalkoxy, wherein each of said alkyl, alkoxy, alkylthio, cycloalkyl, cycloalkoxy, aryl, heterocyclyl, heteroaryl or metallocenoyl may be optionally substituted with C₁₋₅ alkyl, C₁₋₄ alkoxy, C₃₋₆ cycloalkyl, C₁₋₃ haloalkyl, halogen, nitro, amino, hydroxy, oxo, COOH and CONH₂; or —XYZ together represent hydrogen;
R² is Br;
R⁶ is selected from C₁₋₄ alkyl, C₃₋₅ cycloalkyl, C₂₋₄ alkenyl, C₂₋₄ alkynyl, cyano and oxetane;
R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or R^{a} and R^{b} together with the adjacent nitrogen atom form a 3 to 7 membered heterocyclic ring, wherein said heterocyclic ring may be optionally substituted with C₁₋₃ alkyl or halogen; and [symbol]
is a single or double bond or a cyclopropyl ring,
or a salt thereof, said method comprises saponifying bromocriptine followed by amide-formation as shown in the following reaction scheme: 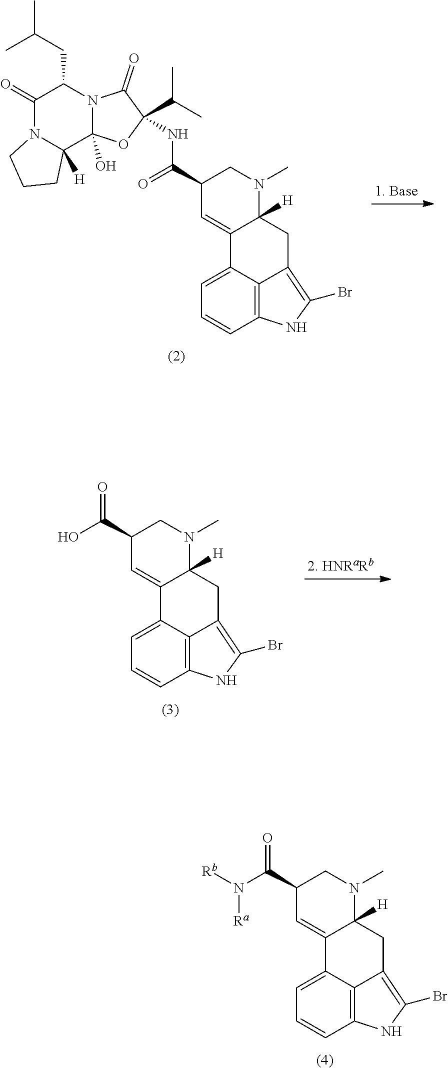
wherein the variables are as defined above.

32. The method of claim 31, wherein the saponification comprises reaction in an aqueous medium with a base in the presence of sodium dithionite and a phase transfer catalyst.

33. The method of claim 31, wherein the amide-formation comprises reaction of the compound of formula (3) with HNR^{a}R^{b} in the presence of a carboxylic acid activating agent.

34. The method of claim 33, wherein the carboxylic acid activating agent is POCl₃.

35. The method of claim 31, which further comprises, for preparing a compound of formula (1) where R⁶ is not methyl, exchanging/extending the residue R⁶ in the compound of formula (3) or (4).

36. The method of claim 31, which further comprises, for preparing a compound of formula (1) where —XYZ is not hydrogen, acylating or sulfonylating the compound of formula (4).

37. A compound of the formula (4) 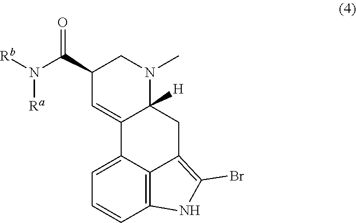
wherein R^{a} and R^{b} are each independently selected from hydrogen, C₁₋₆ alkyl, C₂₋₅ alkenyl, C₂₋₅ alkynyl, C₃₋₇ cycloalkyl, C₂₋₄ haloalkyl, 2-methoxyethyl and 2-ethoxyethyl; or R^{a} and R^{b} together with the adjacent nitrogen atom form a 3 to 7 membered heterocyclyl ring, wherein said heterocyclyl ring may be optionally substituted with C₁₋₃ alkyl or halogen, or a salt thereof.
